(19) United States
(12) Patent Application Publication
(10) Pub. No.: US 2023/0355391 A1
(43) Pub. Date: Nov. 9, 2023

(54) TISSUE GRASPING DEVICES AND RELATED METHODS

(71) Applicants: Raghuveer Basude, Fremont, CA (US); Shri Krishna Basude, Fremont, CA (US); Aishwarya Basude, Fremont, CA (US)

(72) Inventors: Raghuveer Basude, Fremont, CA (US); Shri Krishna Basude, Fremont, CA (US); Aishwarya Basude, Fremont, CA (US)

(21) Appl. No.: 18/245,410

(22) PCT Filed: Mar. 25, 2021
(86) PCT No.: PCT/US21/24255

§ 371 Date: Mar. 15, 2023

Related U.S. Application Data
(60) Provisional application No. 62/994,575, filed on Mar. 25, 2020.
(60) Provisional application No. 63/051,737, filed on Jul. 14, 2020.
(60) Provisional application No. 63/127,935, filed on Dec. 18, 2020.

(51) Int. Cl. A61F 2/24

(52) U.S. Cl. CPC A61F 2/2466; A61F 2220/0075

(57) ABSTRACT

A clip for immobilizing leaflets of a cardiac or venous valve includes a hub having a pair of tangle resistant spring-biased outer arms coupled to an inferior end of the hub and a pair of tangle resistant spring-biased inner arms adjacent to the outer arms and coupled to a superior end of the hub. A delivery catheter with stiffening members configured to position the valve clip adjacent a target valve while the outer and inner arms are biased in an opened position relative to each other. The delivery catheter or clip comprises of sutures that assist in bail out. After the valve leaflets are located between the opened outer and inner arms, the biasing forces may be released to allow the clip to self-close the clip over the valve leaflets, to atraumatically and effectively cinch and coapt the leaflets.

### CROSS-REFERENCE TO RELATED APPLICATIONS

This application claims the benefit of Provisional No. 62/994,575, filed Mar. 25, 2020; Provisional No. 63/051,737, filed: Jul. 14, 2020, and Provisional No. 63/127,935, filed Dec. 18, 2020, the full disclosures of which are incorporated herein by reference.

The disclosure of this is related to those of the following patent publications having common inventorship herewith, the full disclosures of which are incorporated herein by reference: U.S. Application No. 2019/0142589, filed Jan. 14, 2019; PCT Publication No. WO2019143726A1, filed Jan. 16, 2019, PCT Publication No. WO2019010370A1, filed Jul. 6, 2018, PCT Publication No. WO/2019/143726A1, filed Jan. 16, 2019, PCT Application Number PCT/US2017/042003, filed on 13 Jul. 2017; and PCT Publication WO/2019/209871, filed on 23 Apr. 2019, PCT Publication No. WO201801856A1, filed Jul. 13, 2017, referred to herein as the commonly owned prior patent application.

### BACKGROUND OF THE INVENTION

### 1. Field of the Invention

The present invention relates generally to medical methods, devices, and systems. In particular, the present invention relates to methods, devices, and systems for the structural heart, endovascular, percutaneous, or minimally invasive surgical treatment of bodily tissues, such as tissue approximation or valve repair. More particularly, the present invention relates to methods and devices for the repair of mitral and tricuspid heart valves, venous valves, and other tissue structure through minimally invasive and other procedures.

### SUMMARY OF THE INVENTION

This invention provides devices, systems and methods for tissue approximation and repair at treatment sites. The devices, systems and methods of the invention will find use in a variety of therapeutic procedures, including structural heart, endovascular, minimally-invasive, and open surgical procedures, and can be used in various anatomical regions, including the abdomen, thorax, cardiovascular system, heart, intestinal tract, stomach, urinary tract, bladder, lung, and other organs, vessels, and tissues. The invention is particularly useful in those procedures requiring minimally invasive or endovascular access to remote tissue locations, particularly those in which the instruments utilized must negotiate long, narrow, and tortuous pathways to the treatment site. In addition, many of the devices and systems of the invention are adapted to be reversible and removable from the patient at any point without interference with or trauma to internal tissues.

The invention enables two or more valve leaflets to be coapted using an “edge-to-edge” or “bow-tie” technique to reduce regurgitation yet does not require open surgery through the chest and heart wall as in conventional approaches. In addition, the position of the leaflets may vary in diseased mitral valves depending upon the type and degree of disease, such as calcification, prolapse or flail. These types of diseases can result in one leaflet being more mobile than the other (e.g. more difficult to capture), and therefore more difficult to grasp symmetrically in the same grasp with the other leaflet. The features of the present invention allow the fixation devices to be adapted to meet the challenges of unpredictable target tissue geometry, as well as providing a more robust grasp on the tissue once it is captured. Additionally, the invention optionally incorporates visualization techniques to enable the device placement procedure to be performed without the use of general anesthesia.

Coaptation of leaflets, Cinching of annulus, and Thromboembolization. The most dominant edge-to-edge repair device is the MitraClip®, sold by Abbott Vascular, Santa Clara, California, USA. Although marketed as edge-to-edge device, the MitraClip® design has a large gap between the opposing arms. Hence, the device does not fully coapt the leaflets at the tip, and thereby, does not fully cinch the annulus. In complete coaptation of leaflets results in suboptimal efficacy in reducing the mitral valve regurgitation (MR). On the other hand, suboptimal cinching of the annulus results in suboptimal reverse remodeling of the heart.

Furthermore, per the MitraClip® IFU, the MitraClip® device is typically closed to a V-shape only. This causes further separation between the leaflets at the tips.

Since the MitraClip® device has bare metal components in between the Arms, the large gap exposes them to the circulating blood, thereby introducing a thromboembolization risk.

In summary, the key drawbacks of the MitraClip® device includes suboptimal coaptation due to large gap between the arm tips, suboptimal cinching of annulus due to large gap and V-shape closing of the arms, and thromboembolization risks due to exposed bare metal components

One another device, the PASCAL, sold by Edwards Lifesciences Corp., Irvine, California, USA, is essentially a hybrid of edge-to-edge and a spacer technique. While this spacer-based design fills the large gap between the leaflets, and hence mitigates the thromboembolization risks, it does suffer from suboptimal coaptation due to large gap between the paddles. This suboptimal coaptation results in suboptimal or no cinching of annulus, and thereby, suboptimal or no reverse remodeling of the heart.

One particular advantage of this invention is robust coaptation forces resulting in complete coaptation of the native leaflets.

One particular advantage of this invention is robust cinching forces resulting in optimal reverse remodeling of the heart.

One particular advantage of this invention is robust coaptation, resulting in complete coaptation of the native leaflets.

One particular advantage of this invention is robust cinching, resulting in optimal reverse remodeling of the heart.

One particular advantage of this invention is minimal to no exposed bare metal components to circulating blood, mitigating potential thromboembolization risk.

Dynamic and or Progressive Cinching of Annulus

There can be a patient disease condition wherein, cinching the annulus acutely can result in leaflet tears. One advantage of this invention is incorporation of an adjustable or dynamic spacer 690 in between the two grippers, to progressively coapt the leaflets and thereby progressively cinch the annulus over a period of time (chronically).

Bailout Sutures and Wider Leaflet Grasping

In previous referenced and co-owned applications such as WO201801856A1 and WO/2019/143726A1, bailout using sutures were described. Bailout using sutures mitigate the risks of having complex implant designs.

In this invention, additional methods of bailout using bailout sutures are described.

One exemplary embodiment of this invention is a method of bailout suture that interacts with delivery catheter components only and has no direct attachment to the implant.

One alternate exemplary embodiment of this invention is a method of bailout suture that interacts with both delivery catheter component and the Implant.

One alternate exemplary embodiment of this invention is simplified bailout system, wherein, the bailout suture is part of the implant.

Remote Controlled Steerability of the Catheter and Actuation of the Implant

Exemplary embodiments of this invention comprise of automated, remote, electric, microprocessor based, electronic, software controlled, tele-controlled, pre, during, and/or post-procedure actuation of the implant and/or catheter.

Exemplary embodiments of this invention comprise of automated, remote, electric, microprocessor based, electronic, software controlled, tele-controlled, pre, during, and/or post-procedure steering of the delivery system or catheter.

Exemplary embodiments of this invention comprise of automated, remote, electric, microprocessor based, electronic, software controlled, tele-controlled, pre, during, and/or post-procedure actuation of the implant and/or catheter, using nitinol motor or similar actuator.

Exemplary embodiments of this invention comprise of Transcatheter Edge-to-Edge Repair (TEER) systems for treating mitral valve regurgitation via femoral and/or jugular vein access.

Exemplary embodiments of this invention comprise of TEER systems for treating tricuspid valve regurgitation via femoral and/or jugular vein access.

Adjustable, Static, Dynamic Spacer

Exemplary embodiments of this invention comprise of automated, remote, electric, microprocessor based, electronic, software controlled, tele-controlled, manual, pre, during, and/or post-procedure inflation or deflation of the spacers using self-sealing seals and removable tethers.

Exemplary embodiments of this invention comprise of automated, remote, electric, microprocessor based, electronic, software controlled, tele-controlled, manual, pre, during, and/or post-procedure inflation or deflation of the dynamic spacers using external and/or implantable pumps.

Exemplary embodiments of this invention comprise of pre, during, and/or post-procedure inflation or deflation of the dynamic spacers using automated, remote, electric, microprocessor based, electronic, software controlled, tele-controlled, manual, external and/or implantable pumps.

Exemplary embodiments of this invention comprise of various methods of spacers configured to mitigate thromboembolization risks.

Exemplary embodiments of this invention comprise of various methods of spacers configured to mitigate valve regurgitation.

Exemplary embodiments of this invention comprise of various methods of spacers configured to mitigate valve regurgitation, when attached to both leaflets.

Exemplary embodiments of this invention comprise of various methods of spacers configured to mitigate valve regurgitation, when attached to single leaflet.

Atraumatic Barbs, Frictional Elements, Grippers

Exemplary embodiments of this invention comprise of various designs of barbs configured to mitigate leaflet trauma and/or tear risks.

Exemplary embodiments of this invention comprise of various designs of barbs configured to mitigate chordae trauma, tear, rupture, and/or entanglement risks.

Valve Replacement Device with Leaflet Grasping Arms and/or Grippers with Bailout

Most current solutions for valve replacement systems do not have dynamically or manually actuatable leaflet grasping features. Typically, they are either passive barbs or at time elongated barbs that can to be actively controlled to engage or disengage with the leaflets. Hence, once engaged, they cannot be disengaged easily to bailout or retract the device. This leads to problems such as:
Need to disengage often requires the entire device to be retracted into the catheter
Much earlier ‘point of no return’ in the implantation procedure, forcing suboptimal deployment

Exemplary embodiments of this invention comprise of the incorporating into the valve replacement devices the proven methods of leaflet grasping arms and/or gripper, similar to TEER devices.

One particular advantage of this hybrid system is robust ‘point of no return’ that allows for multiple grasping or leaflet engaging attempts during the prosthetic valve implantation.

One particular advantage of this hybrid system is robust grasp or leaflet engagement to mitigate valve replacement device migration.

One particular advantage of this hybrid system is robust grasp or leaflet engagement, resulting in a smaller or less obstructive valve replacement device design.

Ergonomic, Single User, Small Profile 2-Catheter or 3-Catheter Delivery System

MitraClip® is delivered via a 25F, 3-catheter system that is unergonomic, difficult to use, and has a long learning curve. Similarly, Pascal is delivered via 22F, 3-catheter system. Per literature, a 22F catheter have about 50% persistent rate of iatrogenic atrial septal defect (iASD). Profile sizes 14-20F have iASD rate of about 23% and 12F have about 6.8%.

One particular advantage of the invention is a simple, intuitive, easy to use, 12 or 14F, 2-catheter system, to deliver the TEER device via femoral or jugular vein.

One particular advantage of the invention is a TEER delivery system that can be implanted by a single user/operator.

In one exemplary embodiment, the complex multiplane curved are achieved using a combination of stiffening members and wires.

In one exemplary embodiment, the proximal curve in the right atrium/SVC is limited to single plane by using a stiffening member that allows flexion in plane while prevents bending out of plane.

In one exemplary embodiment, the proximal curve in the right atrium/SVC is limited to single plane by using a stiffening member can be made of rectangular flat wire that easily bends about the thickness, however, resists bending about the width, due to anisotropic moment of inertial.

In one exemplary embodiment, the proximal curve in the right atrium/SVC is limited to single plane by using a stiffening member can be made of rectangular flat wire that easily bends about the thickness, however, resists bending about the width, due to anisotropic moment of inertial.

In one exemplary embodiment, the proximal curve in the right atrium/SVC is steered using a flat rectangular wire, to provide both steering as well as function as a stiffening member.

In one exemplary embodiment, the proximal curve in the right atrium/SVC is steered using a flat rectangular wire, to provide both steering as well as function as a stiffening member.

In one exemplary embodiment, one-way or 2-way in plane steering in the right atrium/SVC (proximal steerable segment) is achieved using a flat rectangular wire, to provide both steering as well as function as a stiffening member, while 3-way or 4-way steering in orthogonal planes is achieved using round wires in the left atrium (distal steerable segment).

One advantage of this invention is the flat plane of the delivery catheter handle matches the flat plane of the Implant, wherein, each pair of the Actuator Rod-Arm 597 and Actuator Rod-Gripper 598, intuitively align with their corresponding pair of Implant Arm and Gripper.

Retrieval, Bailout, Funnels, Coils, Fans, Guides

One problem with large devices such as MitraClip® is that retraction of the device into the guide catheter during bailout is often not easy. One advantage of this invention is an expandable funnel that helps direct the device inside the guide catheter.

In one exemplary embodiment, the expandable funnel is part of the guide catheter.

In one exemplary embodiment, the expandable funnel is part of the delivery catheter.

In one exemplary embodiment, the expandable funnel is part of the rescue catheter.

As some exemplary embodiments, the function of directing to safely retract and remove the implant form the body during bailout is achieved by using a funnel, coil, fan, and/or a balloon feature at the distal tip of the catheter and/or balloon feature just proximal to the implant.

One advantage of this invention is that the expandable implant retraction features allow for unrestricted distal delivery catheter segment 615 for easy insertion/passage of the device across leaflets or obstructions.

The following numbered clauses describe other examples, aspects, and embodiments of the inventions described herein:

1. COAPTATION OF LEAFLETS, CINCHING OF ANNULUS, AND THROMBOEMBOLIZATION CLAUSES

2. A method for clipping an anatomical valve, said method comprising:
advancing a valve clip having a plurality of expandable spacers, a pair of outer arms and a pair of inner arms to a location adjacent to the anatomical valve;
biasing at least one of (1) the pair of outer arms and (2) the pair of inner arms to open a valve leaflet capture space between adjacent outer and inner arms;
positioning the valve clip so that one valve leaflet is positioned in the valve leaflet capture space between left outer and inner arms and another valve leaflet is positioned in the valve leaflet capture space between right outer and inner arms; and;
releasing bias on the at least one pair of outer or inner arms so that the left outer and inner arms and the right outer and inner arms self-close over and secure the valve leaflets; and expanding the spacers.

3. A method of Clause 2 wherein, the spacers are expanded in the gap in between of the leaflets, device, and or tissue.

4. A method of Clause 2 wherein, the spacer volume can be dynamically controlled by an electromechanical pump.

5. A method of Clause 2 wherein, the spacer volume can be adjustable using a detachable tethered during or post procedure.

6. A method of Clause 2 to 5 wherein, the spacers are expanded to fill gaps to prevent thromboembolism.

7. A method of Clause 2 to 5 wherein, the spacers are expanded to support leaflets

8. A method of Clause 2 to 5 wherein, the spacers are expanded to fill regurgitant gaps between the leaflets.

9. A method of Clause 2 to 8 wherein, the Outer arms are inclined or bent at the tip as in exemplary FIGS. 54 and 55, to coapt the leaflets with minimal or no gap.

10. A method of Clause 2 to 9 wherein, the Outer arms are elastically flexible at the tip as in exemplary FIG. 56, to coapt the leaflets with robust force.

11. A method of Clause 2 to 9 wherein, the Outer arms are bent at the tip as in exemplary FIGS. 54 and 55, to coapt the leaflets with a robust force.

12. A method of above or below clauses wherein, the gap between the leaflets is preferably <1 mm or between 0, 0.5, 1, 1.5, 2, 2.5, 3, 3.5, 4, . . . 19.5, and/or 20 mm.

13. A method of above or below clauses wherein, the robust coapting force between the leaflets is preferably <0.51bf or between 0, 0.25, 0.5, 0.75, 1, 1.25, 1.5, 1.75, 2, 2.25, 2.5, 2.75, 3, 3.25, 3.5, 3.75, . . . 49.75, and/or 501bf.

14. A method of above or below clauses wherein, the thickness of the outer arm is preferably about 0.33 mm or between 0.01, 0.1, 0.12, 0.14, 0.16, 0.18, 0.2, 0.22, 0.24, 0.26, 0.28, 0.3, 0.32, 0.34, 0.36, 0.38, 0.4, 0.42, . . . , 9.8, and/or 10 mm.

15. A method of above or below clauses wherein, the thickness of the inner arm is preferably about 0.20 mm or between 0.01, 0.1, 0.12, 0.14, 0.16, 0.18, 0.2, 0.22, 0.24, 0.26, 0.28, 0.3, 0.32, 0.34, 0.36, 0.38, 0.4, 0.42, . . . , 9.8, and/or 10 mm.

16. A method of above or below clauses wherein, the width of the outer arm is preferably about 2.1 mm or between 0, 0.5, 1, 1.5, 2, 2.5, 3, 3.5, 4, . . . 19.5, and/or 20 mm.

17. A method of above or below clauses wherein, the width of the inner arm is preferably about 2.1 mm or between 0, 0.5, 1, 1.5, 2, 2.5, 3, 3.5, 4, . . . 19.5, and/or 20 mm.

18. A method of the above or below clauses wherein, the maximum length of leaflet captured between a pair of outer and inner arms is preferably between 5 mm and 20 mm, or between 0.25, 0.5, 1, 1.5, 2, 2.5, 3, 3.5, 4, . . . 59.5, and/or 60 mm.

19. An endovascular heart valve repair system comprising:
a delivery catheter having a distal end configured to be introduced into a heart chamber adjacent to a pair of coapting heart valve leaflets, said delivery catheter including a release bar having a pair of inverters;
a valve repair leaflet grasping device comprising a hub configured to be removably attached to the release bar of the delivery catheter, a first pair of leaflet capture arms comprising a first inner arm and a first outer arm coupled to the hub, and a second pair of leaflet capture arms comprising a second inner arm and a second outer arm coupled to the hub; and
a first set of control tethers positioned on or through the delivery catheter and coupled to the outer arms and configured to selectively bias the outer arms into a valve leaflet capture position; and
a second set of tethers positioned on or through the delivery catheter and coupled to the inner arms and configured to selectively bias the inner arms into a valve leaflet capture position;
wherein the first set of control tethers are threaded through laterally spaced-apart locations on the inverters so that drawing proximal portions of the of the first set of control tethers in a proximal direction causes distal portions of first set of control tethers to pull outer segments of the outer arms in a distal direction into the valve leaflet capture position.

20. A device of clause 19, comprising of a single pair of inner arm and outer arm, and corresponding single inverter.

21. A device of clauses 19 and 20, comprising of expandable/compressible spacers.

22. A device of clauses 19 and 20, configured to fill gaps and/or provide support to the leaflets.

23. A device of above or below clauses wherein, the spacers are expanded in the gap in between of the leaflets, device, and or tissue.

24. A device of above or below clauses wherein, the spacer volume can be dynamically controlled by an electromechanical pump.

25. A device of above or below clauses wherein, the spacer volume can be adjustable using a detachable tethered during or post procedure.

26. A device of above or below clauses wherein, the spacers are expanded to fill gaps to prevent thromboembolism.

27. A device of above or below clauses wherein, the spacers are expanded to support leaflets.

28. A device of above or below clauses wherein, the spacers are expanded to fill regurgitant gaps between the leaflets.

29. A device of above or below clauses wherein, the Outer arms are inclined or bent at the tip as in exemplary FIGS. 54 and 55, to coapt the leaflets with minimal or no gap.

30. A device of above or below clauses wherein, the Outer arms are elastically flexible at the tip as in exemplary FIG. 56, to coapt the leaflets with robust force.

31. A device of above or below clauses wherein, the Outer arms are bent at the tip as in exemplary FIGS. 54 and 55, to coapt the leaflets with a robust force.

32. A device of above or below clauses wherein, the gap between the leaflets is preferably <1 mm or between 0, 0.5, 1, 1.5, 2, 2.5, 3, 3.5, 4, . . . 19.5, and/or 20 mm.

33. A device of above or below clauses wherein, the robust coapting force between the leaflets is preferably <0.51bf or between 0, 0.25, 0.5, 0.75, 1, 1.25, 1.5, 1.75, 2, 2.25, 2.5, 2.75, 3, 3.25, 3.5, 3.75, . . . 49.75, and/or 501bf.

34. A device of above or below clauses wherein, the thickness of the outer arm is preferably about 0.33 mm or between 0.01, 0.1, 0.12, 0.14, 0.16, 0.18, 0.2, 0.22, 0.24, 0.26, 0.28, 0.3, 0.32, 0.34, 0.36, 0.38, 0.4, 0.42, . . . , 9.8, and/or 10 mm.

35. A device of above or below clauses wherein, the thickness of the inner arm is preferably about 0.20 mm or between 0.01, 0.1, 0.12, 0.14, 0.16, 0.18, 0.2, 0.22, 0.24, 0.26, 0.28, 0.3, 0.32, 0.34, 0.36, 0.38, 0.4, 0.42, . . . , 9.8, and/or 10 mm.

36. A device of above or below clauses wherein, the width of the outer arm is preferably about 2.1 mm or between 0, 0.5, 1, 1.5, 2, 2.5, 3, 3.5, 4, . . . 19.5, and/or 20 mm.

37. A device of above or below clauses wherein, the width of the inner arm is preferably about 2.1 mm or between 0, 0.5, 1, 1.5, 2, 2.5, 3, 3.5, 4, . . . 19.5, and/or 20 mm.

38. A device of above or below clauses wherein, the width of the inner or outer arm expands to increase the width of leaflet capture, preferably by about 3 mm or between 0, 0.5, 1, 1.5, 2, 2.5, 3, 3.5, 4, . . . 19.5, and/or 20 mm.

39. A device of the above or below clauses wherein, the maximum length of leaflet captured between a pair of outer and inner arms is preferably between 5 mm and 20 mm, or between 0.25, 0.5, 1, 1.5, 2, 2.5, 3, 3.5, 4, . . . 59.5, and/or 60 mm.

40. A variation of a device of above or below clauses, comprising of a single pair of outer arms and inner arms, wherein, a single leaflet is captured between the arms.

41. A variation of a device of above or below clauses, comprising of at least a pair of outer arms only (no inner arms), wherein the pair of outer arms are configured to be biased apart to create a tissue capture space there between and to resiliently self-close over a leaflet when unbiased after the leaflet has been captured/grasped.

42. A variation of a device of above or below clauses, comprising of at least a pair of outer arms only (no inner arms), wherein the pair of outer arms are configured to be biased apart to create a leaflet capture space there between and to resiliently self-close over at least two leaflets when unbiased after the leaflets have been captured/grasped.

43. A device of the above or below clauses, wherein the device robustly coapts the leaflets with a force larger than the opposing in-vivo forces.

44. A device of the above or below clauses, wherein the device cinches the annulus.

45. A device of the above or below clauses, wherein the device cinches the annulus preferably between 1 mm to 6 mm, or between 0, 0.5, 1, 1.5, 2, 2.5, 3, 3.5, 4, . . . , 59.5, and/or 60 mm.

46. VALVE REPLACEMENT

47. An expandable valve replacement device comprising:
a first pair of tissue grasping arms comprising a first inner arm and a first outer arm coupled to the device; and
a second pair of tissue grasping arms comprising of a second inner arm and a second outer arm coupled to the device; and
a scaffold comprising of prosthetic valves, and said scaffold configured to expand from a crimped configuration to and expanded configuration;
wherein each pair of outer and inner arms are configured to be biased apart to create a tissue capture space there between and to resiliently self-close over the tissue when unbiased after the tissue has been captured/grasped; and the said scaffold is expanded after the tissue has been captured by each pair of arms.

48. An endovascular heart valve replacement system comprising:
a delivery catheter having a distal end configured to be introduced into a heart chamber adjacent to a pair of coapting heart valve leaflets, said delivery catheter including a release bar having a pair of inverters;
a valve repair leaflet grasping device comprising a hub configured to be removably attached to the release bar of the delivery catheter, a first pair of leaflet capture arms comprising a first inner arm and a first outer arm coupled to the hub, and a second pair of leaflet capture arms comprising a second inner arm and a second outer arm coupled to the hub; and
a first set of control tethers positioned on or through the delivery catheter and coupled to the outer arms and configured to selectively bias the outer arms into a valve leaflet capture position; and
a second set of tethers positioned on or through the delivery catheter and coupled to the inner arms and configured to selectively bias the inner arms into a valve leaflet capture position;
wherein the first set of control tethers are threaded through laterally spaced-apart locations on the inverters so that drawing proximal portions of the of the first set of control tethers in a proximal direction causes distal portions of first set of control tethers to pull outer segments of the outer arms in a distal direction into the valve leaflet capture position and further drawing in proximal direction pulls the outer segments of the arms in inverted position.

49. A method for replacing an anatomical valve with a prosthetic valve, said method comprising:
advancing a prosthetic valve device having at least a pair of outer arms and a pair of inner arms to a location adjacent to the anatomical valve;
biasing at least one of (1) the pair of outer arms and (2) the pair of inner arms to open a valve leaflet capture space between adjacent outer and inner arms;
positioning the valve clip so that one valve leaflet is positioned in the valve leaflet capture space between the first pair outer and inner arms and another valve leaflet is positioned in the valve leaflet capture space between second pair of outer and inner arms; and;
releasing bias on the at least one pair of outer or inner arms so that the first pair of outer and inner arms and the second pair of outer and inner arms self-close over and secure the native valve leaflets; and
repeating the native valve leaflet capture sequence if needed; and
expanding the prosthetic valve device after the leaflets have been captured.

50. A variation of above or below device and method clauses of a valve replacement device, comprising of a single pair of inner and outer arms.

51. A variation of above or below device and method clauses of a valve replacement device, comprising of multiple pairs of inner and outer arms to capture the same of more leaflets.

52. A variation of above or below device and method clauses of a valve replacement device, comprising of at least one outer arm, wherein, the tissue/leaflets are captured in the space between the arm and the prosthetic valve device.

53. A variation of above or below device and method clauses of a valve replacement device, comprising of at least one expandable spacer to prevent perivalvular leakage.

54. A variation of above or below device and method clauses of a valve replacement device, comprising of at least one spacer with adjustable volume.

55. A variation of above or below device and method clauses of a valve replacement device, comprising of at least one spacer that can be adjusted dynamically, remotely, electronically, manually or automatically.

56. A device of above or below clauses wherein, the thickness of the outer arm is preferably about 0.33 mm or between 0.01, 0.1, 0.12, 0.14, 0.16, 0.18, 0.2, 0.22, 0.24, 0.26, 0.28, 0.3, 0.32, 0.34, 0.36, 0.38, 0.4, 0.42, . . . , 9.8, and/or 10 mm.

57. A device of above or below clauses wherein, the thickness of the inner arm is preferably about 0.20 mm or between 0.01, 0.1, 0.12, 0.14, 0.16, 0.18, 0.2, 0.22, 0.24, 0.26, 0.28, 0.3, 0.32, 0.34, 0.36, 0.38, 0.4, 0.42, . . . , 9.8, and/or 10 mm.

58. A device of above or below clauses wherein, the width of the outer arm is preferably about 2.1 mm or between 0, 0.5, 1, 1.5, 2, 2.5, 3, 3.5, 4, . . . 19.5, and/or 20 mm.

59. A device of above or below clauses wherein, the width of the inner arm is preferably about 2.1 mm or between 0, 0.5, 1, 1.5, 2, 2.5, 3, 3.5, 4, . . . 19.5, and/or 20 mm.

60. A device of the above or below clauses wherein, the maximum length of leaflet captured between a pair of outer and inner arms is preferably between 5 mm and 20 mm, or between 0.25, 0.5, 1, 1.5, 2, 2.5, 3, 3.5, 4, . . . 59.5, and/or 60 mm.

61. A device of the above or below clauses wherein, the maximum length of leaflet captured by an arm is preferably between 5 mm and 20 mm, or between 0.25, 0.5, 1, 1.5, 2, 2.5, 3, 3.5, 4, . . . 59.5, and/or 60 mm.

62. GENERAL CLAUSES

63. A device or method as shown in any of the figures in this application.

64. A device or method that can be derived from a combination of any of the figures in this application.

65. A device or method as described or explained in in this application.

66. A device or method that can be derived from a combination of any of the description or explanation in this application.

67. An expandable arm as shown in or derived from any of the FIGS. 1A to 7B.

68. A Release Bar as shown in or derived from any of the FIGS. 8A to 8B.

69. A Bailout system or method as shown in or derived from any of the FIGS. 9A to 12G.

70. A Bailout system or method as shown in FIG. 9B.

71. A Bailout system or method as shown in FIG. 10B.

72. A Bailout system or method as shown in FIG. 12D.

73. A spacer device or method as shown in FIG. 27.

74. A spacer device or method as shown in FIG. 28.

75. A spacer device or method as shown in FIG. 29.

76. A spacer device or method as shown in FIG. 30.

77. A spacer device or method as shown in FIG. 31.

78. A spacer device or method as shown in FIGS. 32, 33, 34, 35, 36, and/or 37.

79. A spacer device or method as shown in or derived from any of the FIGS. 27 to 27.

80. A device or method as shown in FIG. 38, configured to provide robust tissue coaptation and or annulus cinching.

81. A device or method as shown in FIGS. 38, 39, 40, 41, 42, 43 and/or 44.

82. A leaflet grasping device or method comprising of a spacer, as shown in FIG. 53.

83. A tissue grasping device or method as shown in FIG. 54, 55, 56, 57, 58, 59, 60, 61, 62, 63, 64, and or 65.

84. A tissue grasping device or method as shown in FIG. 66, 67, 68, 69, 70, and or 71.

85. A tissue grasping device or method as shown in FIG. 72, 74, 75, and or 76.

86. A tissue grasping device or method as shown in FIG. 78, and or 79.

87. A tissue grasping device comprising of a leaflet grasping arm with atraumatic barbs as shown in FIGS. 81, 82, and/or 83.

88. A tissue grasping device or method as shown in FIG. 84 and or 85.

89. A release bar as shown in FIG. 86.

90. A tissue grasping device or method as shown in FIG. 87, 88, 89, 90, 91, 92, 93, 94, 95, and or 96.

91. A tissue grasping system or method as shown in FIG. 97, 98, and or 99.

92. A Release Bar as shown in FIG. 100.

93. A valve replacement device or method as shown in FIG. 102, 103, 104, 105, 106, 107, and or 108.

94. A valve replacement device or method as shown in FIG. 102, 103, 104, 105, 106, 107, and or 108, comprising of an expandable spacer configured seal paravalvular leaks.

95. A catheter delivery system for a valve repair or replacement device or method as shown in FIG. 109A, 109B, 109C, and or 109D.

96. A steerable guide catheter for a valve repair or replacement device or method comprising of stiffening members or stiffening pull-wires as shown in FIG. 110, 111, 112, 113, and or 114.

97. A delivery catheter handle for a valve repair or replacement device or method as shown in FIG. 115, 116, 117, 118, and or 119.

98. A valve repair or replacement implant retracting or retrieval feature/component or method as shown in FIG. 120, 121, 123, 124, and or 125.

99. A valve repair or replacement implant retracting or retrieval feature/component or method as shown in FIG. 126, and or 127.

100. A delivery catheter comprising of an external spring 625 in the distal segment, configured to reduce friction and maintain straightness when it is advanced out of the guide catheter, as shown in FIG. 128.

101. A rescue catheter or method as shown in FIG. 129A, 129B, 129C, and or 129D.

102. A tissue grasping device or method as shown in FIG. 130A and or 130B.

103. PROVISIONAL NO. 62/994,575 CLAUSES

104. A tissue grasping device comprising: a hub configured to be removably attached to the deployment shaft; a first pair of tissue grasping arms comprising a first inner arm and a first outer arm coupled to the hub; and a second pair of tissue grasping arms comprising of a second inner arm and a second outer arm coupled to the hub; wherein each pair of outer and inner arms are configured to be biased apart to create a tissue capture space therebetween and to resiliently self-close over the tissue when unbiased after the tissue has been captured/grasped. The deployment shaft or the tissue grasping device have actuatable features configured to expel the leaflets to allow for bailout.

105. The tissue grasping device of clause 1, wherein the hub is a metal tube; wherein, the actuatable features are comprised of implantable and or removable sutures (wires, leafsprings, fabric, and or rope); wherein, the actuation comprises of manual, electrical, thermal, chemical, and or mechanical.

106. The tissue grasping device of clause 1, wherein the outer and inner arms are comprised of metal wires.

107. The tissue grasping device of clause 1, wherein the pair of tissue grasping arms is a singular component, such that the inner arm is a part of the outer arm.

108. The tissue grasping device of clause 4, wherein the tissue grasping arm is produced from metal strips, metal tubes, sheet metal and/or any other flexible material suitable for implantation in the human body.

109. The tissue grasping device of clause 4, wherein the inner arm is configured to be biased apart from the outer arm.

110. The tissue grasping device of clause 4, wherein the outer arm is configured to bend 270 degrees.

111. The tissue grasping device of clause 5, wherein the width of the inner arm can be modified within the range of the circumference of the metal tube.

112. The tissue grasping device of clause 8, wherein the inner arm is comprised of thin, expandable metal sheets with strength pattern to form a folding fan-like design.

113. The tissue grasping device of clause 8, wherein the hub can be comprised of a stent pattern such as peak-to-valley, mid-strut connector, peak-to-peak and/or offset peak-to-peak, and/or strength pattern such as rectilinear, grid, triangular, wiggle, fast honeycomb and/or full honeycomb.

114. The tissue grasping device of clause 9, wherein the inner and/or outer arm can have stent pattern such as peak-to-valley, mid-strut connector, peak-to-peak and/or offset peak-to-peak, and/or strength pattern such as rectilinear, grid, triangular, wiggle, fast honeycomb and/or full honeycomb.

115. The tissue grasping device of clause 9, wherein the inner arm can be formed from flexible material that may be a metal fabric, such as a mesh, woven, braided, or formed in any suitable way or a laser cut or otherwise cut flexible material. The flexible material may be a cloth, shape-memory alloy wire to provide shape setting capability, or any other flexible material suitable for implantation in the human body.

116. The tissue grasping device of clause 9, wherein the inner arms may be biased inward.

117. The tissue grasping device of clause 9, wherein the inner arms may be biased outward.

118. A tissue grasping device, further comprising: a hub configured to be removably attached to the deployment shaft; a first pair of tissue grasping arms comprising a first fixed inner arm connected to a first movable outer arm coupled to the hub; and a second pair of tissue grasping arms comprising of a second fixed inner arm connected to a second movable outer arm coupled to the hub; wherein each pair of outer and inner arms are configured to be biased apart to create a tissue capture space therebetween and to resiliently self-close over the tissue when unbiased after the tissue has been captured/grasped; wherein the movable outer arms are movable between open and closed positions relative to the fixed inner arms.

119. The tissue grasping device of clause 15, wherein outer arms are configured to have a leaflet ejection feature that releases the mitral valve leaflets without inversion.

120. The tissue grasping device of clause 15, wherein the outer arms are coupled by a metal wire and/or suture to a spring-loaded base.

121. The tissue grasping device of clause 17, wherein the suture is extended from the catheter and is attached through eyelets along the perimeter of the movable outer arms.

122. The tissue grasping device of clause 17, wherein the suture is extended from the catheter and is attached to a tethering line along the perimeter of the outer arms

123. The tissue grasping device of clause 17, wherein the suture is extended from the catheter and is attached to a pull/push mechanism coupled to the base of the movable outer arms.

124. The tissue grasping device of clause 17, wherein the spring-loaded base can be actuated by a metal wire/mandrel and/or suture to retract the outer arms up and release the mitral valve leaflets.

125. The tissue grasping device of clause 17, wherein the suture may be part of the implant or part of the delivery system.

126. The tissue grasping device of clause 15, wherein the base does not have to be spring-loaded.

127. The tissue grasping device of clause 15, wherein the fixed arms consist of a plurality of barbs.

128. The tissue grasping device of clause 24, wherein the barbs can be an angle between 10 degrees to 75 degrees to the fixed inner arms.

129. The tissue grasping device of clause 24, wherein the barbs are angled away from the movable outer arms to prevent excessive pinching or clipping force on the leaflets.

130. The tissue grasping device of clause 15, wherein the fixed inner arms can be biased inward at an angle between 10 degrees and 350 degrees from the movable outer arms.

131. The tissue grasping device of clause 15, wherein the movable outer arms can be biased outward at an angle between 10 degrees and 350 degrees from the movable outer arms.

132. A method for releasing the mitral valve leaflets of a patient for bailout and repositioning without inversion, the method comprising: applying tension in a pulling motion to the release suture to retract the movable outer arms upwards and thereby moving the fixed inner arms; releasing the barbs from the mitral valve leaflets; retracting the delivery system; and actuating the release suture to move the outer arms in a open or closed position.

133. The method of clause 29, wherein the release suture can be a metal wire, metal shaft, metal rod, polymeric suture, and so forth.

134. The method of clause 29, wherein the release suture is coupled to a hub.

135. The method of clause 29, wherein the release suture is not coupled to a hub.

136. The method of clause 29, wherein the release suture is coupled to the movable outer arms.

137. The method of clause 29, wherein the release suture is not coupled to the movable outer arms.

138. The method of clause 29, wherein the release suture is coupled to a pull/push mechanism attached to the base of the movable outer arms.

139. The method of clause 29, wherein the release suture is not coupled to a pull/push mechanism attached to the base of the movable outer arms.

140. The method of clause 29, wherein applying tension to the release suture in a pulling motion will retract the movable outer arms to move the fixed inner arms and thereby release the barbs from the valve leaflets.

141. A tissue grasping device, further comprising: a hub configured to be removably attached to the deployment shaft; a first pair of tissue grasping arms comprising a first fixed inner arm connected to a first movable outer arm coupled to the hub; and a second pair of tissue grasping arms comprising of a second fixed inner arm connected to a second movable outer arm coupled to the hub; wherein each pair of outer and inner arms are configured to contain an automatic bailout feature.

142. The tissue grasping device of clause 38, wherein the automatic bailout feature is looped and/or threaded through the inner and outer arm, thereby creating a suture between the arms that is pulled taut when the outer arms are inverted and expunging any trapped tissue and/or chordae during leaflet capture.

143. The tissue grasping device of clause 39, wherein the bailout suture is comprised of polyester thread, elastic material and/or wire.

144. A system for delivering a tissue grasping device to the heart or venous valve, said device comprising: a tissue grasping device and a deployment shaft configured to be removably attached to the hub of the tissue grasping device.

145. The system of clause 41, wherein the deployment shaft is comprised of a bailout feature.

146. The system of clause 42, wherein the bailout feature is comprised of a bailout suture and a secondary suture.

147. The system of clause 42, wherein the bailout feature is designed such that retraction of the secondary suture slackens the bailout suture, thereby allowing leaflet capture.

148. The system of clause 42, wherein the bailout feature is designed such that retraction of the bailout suture slackens the secondary suture, thereby expunging caught tissue and/or chordae from the tissue grasping arms.

149. The system of clause 42, wherein the bailout feature is designed such that retraction of the bailout suture slackens the secondary suture, thereby releasing the leaflets from the tissue grasping arms without inverting the outer arms.

150. The system of clause 42, wherein the bailout and secondary sutures are made of polyester thread, elastic material and/or wire.

151. The system of clause 43, wherein the bailout suture is threaded through suture loops positioned at the inverters and/or suture loops positioned at the first opening of the release bar.

152. The system of clause 43, wherein the secondary suture is configured to loop through any and/or all of the openings on the release bar.

153. A tissue grasping device of clauses 1-49, wherein said device is comprised of a polyester fabric that is coated with a polymer.

154. A tissue grasping device of clauses 1-49, wherein said device is comprised of a polyester fabric that is not coated with a polymer.

155. A tissue grasping device of clauses 1-49, wherein said device is comprised of a fabric that may be polyester or any biocompatible material suitable for implantation in the human body.

156. A tissue grasping device of clauses 1-49, wherein said device is comprised of a fabric that may be woven, braided and/or knitted.

157. A tissue grasping device of clauses 1-49, wherein said device may be comprised of external attachments on the inner and/or outer arms such as ring, loop, leafspring, sensors and actuators and/or wire for the purpose of increasing mechanical strength.

158. A tissue grasping device of clauses 1-49, wherein said device can be manually, electrically, chemically and/or mechanically actuated.

159. A tissue grasping device of clauses 1-49, wherein said delivery system can be manually, electrically, chemically and/or mechanically actuated.

160. A tissue grasping device of clauses 1-49, wherein said device may be comprised of a single or multiple unit.

161. A tissue grasping device of clauses 1-49, wherein said device may be comprised of a single or plurality of statically, expandable balloons that are inflated by an inflation tube connected to the delivery catheter.

162. A tissue grasping device of clauses 1-49, wherein said device may be comprised of a single or plurality of statically expandable balloons that are inflated by an inflation port controlled by a microprocessor, microcontroller, sensors and/or actuators.

163. A tissue grasping device of clauses 1-49, wherein said device may be comprised of a single or plurality of dynamically expandable balloons that are inflated by an inflation tube connected to the delivery catheter.

164. A tissue grasping device of clauses 1-49, wherein said device may be comprised of a single or plurality of dynamically expandable balloons that are inflated by an inflation port controlled by a microprocessor, microcontroller, sensors and/or actuators.

165. A tissue grasping device of clauses 1-49, wherein said first outer arm may be 0.25 mm, 0.5 mm, 0.75 mm, 1 mm, 1.25 mm, etc. longer than the second outer arm.

166. A tissue grasping device of clauses 1-49, wherein said first inner arm may be 0.25 mm, 0.5 mm, 0.75 mm, 1 mm, 1.25 mm, etc. longer than the second inner arm.

167. A tissue grasping device of clauses 1-49, wherein said first outer arm may be 0.25 mm, 0.5 mm, 0.75 mm, 1 mm, 1.25 mm, etc. longer than the first inner arm.

168. A tissue grasping device of clauses 1-49, wherein said second outer arm may be 0.25 mm, 0.5 mm, 0.75 mm, 1 mm, 1.25 mm, etc. longer than the second inner arm.

169. A tissue grasping device of clauses 1-49, wherein said first outer arm may be 0.25%, 0.5%, 0.75%, 1%, 5%, 10%, etc. thicker than the second outer arm.

170. A tissue grasping device of clauses 1-49, wherein said first inner arm may be 0.25%, 0.5%, 0.75%, 1%, 5%, 10%, etc. thicker than the second inner arm.

171. A tissue grasping device of clauses 1-49, wherein said first outer arm may be 0.25%, 0.5%, 0.75%, 1%, 5%, 10%, etc. thicker than the first inner arm.

172. A tissue grasping device of clauses 1-49, wherein said second outer arm may be 0.25%, 0.5%, 0.75%, 1%, 5%, 10%, etc. thicker than the second inner arm.

173. A catheter handle comprising of retractable rods, o-rings, sutures, suture tensioners, wherein, the retractable rods are slidable over compressed o-ring, rods are attached to sutures and or with sutures along with suture tensioners.

174. The handle of clause 70, wherein, the rods are manually or robotically actuated. 175. The handle of clause 70, wherein, the sutures are manually, electrically, mechanically, chemically and or robotically actuated.

176. A catheter comprised cables and shafts that are flexible and torquable such as those made by ASAHI INTECC USA INC.

177. A valve repair system comprising of audio, visual, tactile, rf, wireless feedback

178. PROVISIONAL NO. 63/051,737 CLAUSES

179. A tissue grasping device comprising a first pair of tissue grasping arms comprising a first inner arm and a first outer arm coupled to a hub; and a second pair of tissue grasping arms comprising of a second inner arm and a second outer arm coupled to a hub; wherein each pair of outer and inner arms are configured to be biased apart to create a tissue capture space there between and to resiliently self-close over the tissue when unbiased after the tissue has been captured/grasped. The fully closed MitraClip® implant when the arms are parallel, have an inherent gap between the tips of the arm. Having the inherent gap causes blood elements to be trapped in this zone and experience high shear stresses for a long period result in thrombosis and thromboembolism. The inherent gap can be filled with a spacer to reduce the risk of thrombus formation.

180. The tissue grasping device of clause 1, wherein the inherent gap between the tip of the arm are filled with a biocompatible sponge, which reduces the blood recirculation zone results in reduction in risk of thrombus formation.

181. The tissue grasping device of clause 1, wherein the inherent gap between the tip of the arm are filled with a biocompatible expandable mesh, which reduces the blood recirculation zone results in reduction in risk of thrombus formation.

182. The tissue grasping device of clause 1, wherein the inherent gap between the tip of the arm are filled with a biocompatible balloon, which reduces the blood recirculation zone results in reduction in risk of thrombus formation.

183. The sponge of clause 2 is attached in-between the arms to fill the inherent gap formed after closing the arms.

184. The expandable mesh of clause 3 is attached in-between the arms to fill the inherent gap formed after closing the arms.

185. The balloon of clause 4 is attached in-between the arms to fill the inherent gap formed after closing the arms.

186. The filling of inherent gap in clause 5, 6 and 7 in Medfree system, can form less than 1 mm gap but tissue bridge formed between the implant and the spacer eliminates the risk of thrombus formation.

187. The sponge of clause 2 is attached to the atrial side of the outer arm to increase leaflet support from underneath

188. The expandable mesh of clause 3 is attached to the atrial side of the outer arm to increase leaflet support from underneath

189. The balloon of clause 4 is attached to the atrial side of the outer arm to increase leaflet support from underneath.

190. The tissue grasping device of clause 1, wherein the inherent gap 52 between the tip of the arm are fastened to the atrial side of one gripper using suture, bond, weld, glue, and/or faster.

191. The tissue grasping device of clause 1, wherein the inherent packet is filled with two small spacers 68 where each spacer is fastened to one arm to decrease the gap 12 as shown in FIG. 13 and FIG. 17.

192. When one arm is lowered (deflected) to obtain grasping position, the second arm also tends to move towards the actuating side.

193. The movement of other arm during grasping in clause 12, wherein the center posts on release bar keeps the arms at the center and prevent passing one of the arms to opposite direction during the actuation of the other arm.

194. The tissue grasping device in clause 1, wherein the arms has extra pivot which provides various degree of freedom in the arms.

195. The extra pivot in clause 12, wherein the arms rigidity is increased and providing better grip by increasing the number of pins.

196. The extra pivot in clause 12, wherein the flexible arms provide better holding and flexibility.

197. The extra pivot in clause 12, wherein the flexible arms provide constant elastic spring force.

198. The Gripper design incorporates unique blunted shaped Frictional Elements (FEs) that are medially placed.

199. The blunted barbs in clause 16, wherein the blunted laser flat pattern decreases the tear leaflet tissue.

200. The laser flat pattern barbs in clause 17, can be in W-shape or V shape or curved.

201. The grippers design in clause 18, provides no leaflet tears, perforations and loss of grasp.

202. The pivot arm in clause 12, wherein there is limited movement due to short distance between the pivot and the holding pin.

203. The extra pivot arm in clause 12, wherein U spring is attached in neutral position providing larger mobility of the screw for bigger range of motion.

204. The extra pivot arm in clause 12, the bigger ratio in the spring holding pin results in tighter grip for the arms.

205. The tissue grasping device in clause 1, wherein contain 240.

206. The removable part of delivery system in clause 24, can create an inherent gap when removed.

207. The inherent gap in clause 25, can be filled with sponge, expandable mesh and balloon as in clause 5,6 and 7.

208. The filling spacer in clause 26, can be replaced with a permanent covering inherent gap 241 to fill the inherent gap between the arms as in FIG. 24 and FIG. 25.

209. The tissue grasping device in clause 1, wherein the angle between the Outer Arms can be −90, −60, −45, −30, −15, −10, −5, 0, 5, 10, 15, 20, 25, 30, 45, 60, and/or 90 degrees.

210. The outer arm angle in clause 28, the preferred angle can be between −10 to 30 degrees.

211. The tissue grasping device in clause 1, wherein the Base width>top width.

212. The base and top width in clause 30, can also be Base width=top width.

213. The base and top width in clause 30, can also be base width<top width

214. The base width in clause 30, 31 and 32, wherein the Base width is greater than 0.1%, 1%, 10%, 25%, 50%, 75%, 100%, 150%, 200%, 500%, 1000%, and or 10,000%

215. The base and top width in clause 30, wherein the Base width and/or top width is 0.01 mm, 0.1 mm, 1 mm, 1.5 mm, 2 mm, 2.5 mm, 3 mm, 4 mm, 5 mm, 6 mm, 10 mm, 20 mm, 30 mm, 50 mm, 100 mm and/or 300 mm.

216. A tissue fixation system configured for intravascular delivery and for use in joining mitral valve tissue during treatment of the mitral valve, comprising:
a body;
a first and second distal elements, each formed of a material, including:
a first end pivotally coupled to the body and extending to a second end; and
a tissue engagement surface between the first and second end, the tissue engagement surface being configured to approximate and engage a portion of leaflets of the mitral valve;
   a tissue gipping device formed of a shape-memory material, including:
   a base section; and
a first arm and a second arm, each arm having a distal end coupled to the base section by an arm bend feature, a proximal end extending laterally from the base section, and a furcated section having an opening with a radiused-terminal distal end formed with a first radius in the arm bend feature, a tapered-terminal proximal end formed with a second radius in the first arm, wherein the second radius is smaller than the first radius, and an intermediate portion having a cross-section larger than both the terminal distal end and the tapered-terminal proximal end, the first and second arms being disposed opposite one another and each arm being configured to cooperate with one of the first or second distal elements to form a space for receiving and holding a portion of mitral valve tissue therebetween.

217. A tissue fixation system of clause 38, wherein, the first and second distal elements are made any of the CP or alloys of titanium materials such as: grade1, grade 2, grade 3, grade 4, grade 5, grade 6, grade 23, Ti-6Al-7Nb, Ti-3A1-2.5V, Ti Beta 3/Ti 11.5Mo-6Zr-4.5Sn, Ti Beta C/Ti-3A1-8V-6Cr-4Zr-4Mo or any other titanium alloy that can be implanted.

218. A tissue fixation system of clause 38, wherein, the first and second distal elements are made any of the non-ferromagnetic materials such as: titanium or titanium alloys or any other non-ferromagnetic biomaterial (metal, polymer, and/or ceramic) that can be implanted.

219. A tissue fixation system configured for intravascular delivery and for use in joining mitral valve tissue during treatment of the mitral valve, comprising:
a body;
a base;
a first and second distal elements, each formed of a shape-memory material, including:
a first end coupled to the body and extending to a second end; and
a tissue engagement surface between the first and second end, the tissue engagement surface being configured to approximate and engage a portion of leaflets of the mitral valve;
   a tissue gipping device formed of a shape-memory material, comprising of:
   multiple frictional elements;
   a base section; wherein:
the distal elements are self-biased towards the grippers and vice-versa, and configured to be flexed against the biasing forces to form space for receiving a portion of leaflets and holding the portion of the leaflets between them by self-closing the space when the external force is removed.

220. The tissue fixation system of clauses 38 and 41, wherein tissue engagement surfaces of the distal elements are angled apart at about 90 degrees or more when positioned in an open configuration, and wherein the first and second arms of the tissue gripping device are configured to move from a pre-deployed configuration toward a deployed configuration by moving toward the tissue engagement surfaces, the first and second arms being angled apart at about 90 degrees or more when positioned in the deployed configuration.

221. The tissue fixation system of clauses 38 and 41, wherein tissue engagement surfaces of the distal elements are angled apart at about 120 degrees or more when positioned in a pre-deployed configuration, and wherein the first and second arms of the tissue gripping device are configured to move from a pre-deployed configuration toward a deployed configuration by moving toward the tissue engagement surfaces, the first and second arms being angled apart at about 120 degrees or more when positioned in the deployed configuration.

222. The tissue fixation system of clauses 38 and 41, wherein the shape-memory material of the tissue gripping device is a nickel titanium alloy.

223. The tissue fixation system of clauses 38 and 41, wherein the nickel titanium alloy of the tissue gripping device has a transformation temperature of between about −40, −30, −20, −10, −5 to about 37 degrees C.

224. The tissue fixation system of clauses 38 and 41, wherein the nickel titanium alloy of the tissue gripping device has a transformation temperature of between about −10 to about 10 degrees C.

225. The tissue fixation system of clauses 38 and 41, wherein the tissue gripping device is configured such that upon being positioned in a deployed state against a leaflet of the mitral valve, an arm of the tissue gripping device exerts a force of about 0.10, 0.11, 0.12, 0.13, 0.14, 0.15, 0.20, 0.25, 0.35, 0.4, 0.45, 0.5, 0.75, 1.0, 1.5, 2.0, 2.5, 3.0, 3.5, 4.0, 4.5, 5.0, 6.0, 7.0, 8.0, 9.0, 10, 12, 15, 20, 30, 50, and or 100 pounds against the leaflet.

226. The tissue fixation system of clauses 38 and 41, wherein the arms of the tissue gripping device, upon moving from a pre-deployed configuration to a deployed configuration, deploy to engage the mitral valve tissue against the tissue engagement surfaces of the distal elements while the distal elements are in an open configuration without any proximal movement of the distal elements.

227. The tissue fixation system of clauses 38 and 41, wherein a full length of the arms of the tissue gripping device, upon moving from a pre-deployed configuration to a deployed configuration, engage the mitral valve tissue against the tissue engagement surfaces of the distal elements while the distal elements are in an open configuration.

228. A tissue fixation system of clause 38 and 41, wherein, the tissue gipping device formed of a shape-memory material is expandable in width and/or thickness.

229. A tissue fixation system of clause 38 and 41, wherein, the tissue gipping device is shaped from a material of thickness>0.006″, preferably between 0.0063″ and 0.201″.

230. The tissue fixation system of clause 38 and 41, wherein the tissue gripping device is configured such that upon being positioned in a deployed state against a leaflet of the mitral valve, an arm of the tissue gripping device exerts a force>0.10 pounds against the leaflet, preferably about 0.11 to about 30 pounds.

231. The tissue fixation system of clauses 41, wherein the self-biasing force of the distal elements is more than the self-biasing force of the proximal Gripper elements by about 0.10, 0.11, 0.12, 0.13, 0.14, 0.15, 0.20, 0.25, 0.35, 0.4, 0.45, 0.5, 0.75, 1.0, 1.5, 2.0, 2.5, 3.0, 3.5, 4.0, 4.5, 5.0, 6.0, 7.0, 8.0, 9.0, 10, 12, 15, 20, 30, 50, and or 100 pounds against the leaflet.

232. The tissue fixation system of clauses 38 and 41, wherein, the distal arms close to decrease the gap between them, wherein, the gap is less than 0.1, 0.15, 0.2, 0.25, 0.3, 0.4, 0.5, 1, 2, 3, 4, 5, 6, 10, 15, 20, 30, 50, and/or 100 mm.

233. The tissue fixation system of clauses 38 and 41, wherein, the distal arms close to decrease the gap between them, wherein, the distal arms, grippers and leaflets in between them are in close apposition, as shown in FIG. 32.

234. The tissue fixation system of clauses 38 and 41, wherein, the distal arms flex when closed tightly.

235. The tissue fixation system of clauses 38 and 41, wherein, the distal arms flex when closed tightly and apply elastic closing forces on the leaflets.

236. The tissue fixation system of clauses 38 and 41, wherein, the distal arms flex when closed do not have any substantial gap that creates a pocket in between the leaflets or grippers at the tip.

237. The tissue fixation system of clauses 38 and 41, wherein, the base is fastened together with distal and proximal elements.

238. The tissue fixation system of clauses 38 and 41, wherein, the base is fastened together with distal and proximal elements, using screws, rivets, clamps and/or ropes.

239. The tissue fixation system of clauses 38 and 41, wherein, the base is bonded together with distal and proximal elements in between.

240. The tissue fixation system of clauses 38 and 41, wherein, the base is welded together with distal and proximal elements in between.

241. The tissue fixation system of clauses 38 and 41, wherein, all components materials are non-ferromagnetic.

242. The tissue fixation system of these clauses, wherein, the distal elements self-expand laterally and/or radially when unconstrained.

243. The tissue fixation system of these clauses, wherein, the proximal gripper elements self-expand laterally and/or radially when unconstrained.

244. The tissue fixation system of these clauses, wherein, an expandable element self-expands to fully or partially fill any pockets in between the distal arms, grippers, and/or leaflets.

245. The tissue fixation system of these clauses, wherein, an expandable element can be remotely configured to self-expand to fully or partially fill any pockets in between the distal arms, grippers, and/or leaflets.

246. The tissue fixation system of these clauses, configured to receive an expandable element to fully or partially fill any pockets in between the distal arms, grippers, and/or leaflets.

247. The tissue fixation system of these clauses, configured to receive a variably expandable element to fully or partially fill any adjacent gaps in the leaflets.

248. The tissue fixation system of clause 69, wherein, the expandable member can be remotely configured to receive a variably expandable element to fully or partially fill any adjacent gaps in the leaflets.

249. The tissue fixation system of any of the above or below clauses that can be delivered using a 20, 19, 18, 17, 16, 15, 14, 13, 12, 11, 10, 9, 8, 7, 6, 5, 4, 3, 2, 1, and/or 0.1 Fr lumen guide/introducer catheter system. Preferably the guide/introducer lumen catheter system is between 18 Fr and 9 Fr, and/or preferably 12 Fr.

250. A tissue grasping device comprising: a hub configured to be removably attached to the deployment shaft; a first pair of tissue grasping arms comprising a first inner arm and a first outer arm coupled to the hub; and a second pair of tissue grasping arms comprising of a second inner arm and a second outer arm coupled to the hub; wherein each pair of outer and inner arms are configured to be biased apart to create a tissue capture space therebetween and to resiliently self-close over the tissue when unbiased after the tissue has been captured/grasped. The deployment shaft or the tissue grasping device comprising sutures configured to expel the leaflets to allow for bailout. A catheter with stiffening member configured to steer in a desired direction. An expandable element configured to fill any gap at in between the leaflets.

251. A steerable catheter shaft comprising of one or more stiffening members incorporated in or over a catheter, to allow for specific steerability in a particular curve or direction.

252. A stiffening member of the catheter of the above or below clause made of laser cut tubing, small segments, laser cut strips, wires, sutures, fibers, polymers, ceramics, metals, and/or composites.

253. A steerable catheter shaft of the above or below clauses comprising of stiffening members continuously or intermittently along the length of the shaft.

254. A steerable catheter shaft of the above or below clauses comprising of stiffening members formed by adding or removing material in a given pattern to assist in directional steering.

255. A steerable catheter shaft of the above or below clauses comprising of stiffening members in the proximal 90%, 80%, 70%, 60%, 50%, 40%, 30%, 20%, 10%, 5%, 1% of the shaft.

256. A steerable catheter shaft of the above or below clauses comprising of stiffening members in the distal 90%, 80%, 70%, 60%, 50%, 40%, 30%, 20%, 10%, 5%, 1% of the shaft.

257. A steerable catheter shaft of the above or below clauses comprising of stiffening members in the center 90%, 80%, 70%, 60%, 50%, 40%, 30%, 20%, 10%, 5%, 1% of the shaft.

258. A steerable catheter shaft of the above or below clauses comprising of stiffening members configures to allow preferred steerability to access a heart valve from femoral vein and IVC.

259. A steerable catheter shaft of the above or below clauses comprising of stiffening members configures to allow preferred steerability to access a heart valve from jugular vein and SVC.

260. A steerable catheter shaft of the above or below clauses comprising of stiffening members configures to allow preferred steerability to access a heart valve from femoral artery and via aorta.

261. A steerable catheter shaft of the above clauses comprising of stiffening members configures to allow preferred steerability to access a heart valve from jugular artery via aorta.

262. A steerable catheter shaft of the above clauses comprising of stiffening members configures to allow preferred steerability to access a heart valve.

263. A steerable catheter shaft of the above clauses comprising of stiffening members configured to provide enhanced trackability, torqueability, steerability, pullability, and/or pushability.

264. PROVISIONAL NO. 63/127,935 CLAUSES

265. A prosthetic treatment apparatus for treating a native mitral valve having a native annulus and native leaflets, comprising:
an expandable support having a downstream end configured to be positioned toward the left ventricle, an upstream end configured to be positioned toward the left atrium, and an interior;
a prosthetic valve having at least one leaflet assembly mounted to the expandable support and adapted to allow blood flow in the downstream direction and to block blood flow in the upstream direction; stream portion of the support and at least one elongate member or plurality of elongate members extending outwardly from the support in an upstream direction, wherein the elongated members have sufficient flexibility to deflect inwardly or outwardly relative to the support to accommodate expansion or distortion of the native annulus, and wherein the elongated members are configured to inhibit movement of the support toward the left atrium; and
at least one skirt coupled to a downstream portion of the support and extending around the support, wherein the skirt is oriented on the device as to inhibit blood flow between the prosthetic treatment device and the native valve.
at least one suture or a plurality of sutures coupled to the elongated member and a feature downstream, configured to deflect the elongated members by tensioning or slacking the sutures, and wherein the elongated member deflection can be configured to various positions that allow for repeated stabilizing, grasping and/or release of the native leaflets.

266. A prosthetic treatment apparatus for treating a native mitral valve having a native annulus and native leaflets, comprising:
an expandable support having a downstream end configured to be positioned toward a left ventricle, an upstream end configured to be positioned toward a left atrium, and an interior;
a prosthetic valve having at least one leaflet assembly mounted to the expandable support and adapted to allow blood flow in the downstream direction and to block blood flow in the upstream direction; stream portion of the support and at least one elongate member or plurality of elongate members extending outwardly from the support in an upstream direction, wherein the elongated members have sufficient flexibility to deflect inwardly or outwardly relative to the support to accommodate expansion or distortion of the native annulus, and wherein the elongated members are configured to grasp the native leaflet from the atrial and ventricular sides, to inhibit movement of the support toward the left atrium; and
at least one skirt coupled to a downstream portion of the support and extending around the support, wherein the skirt is oriented on the device as to inhibit blood flow between the prosthetic treatment device and the native valve.

267. at least one suture or a plurality of sutures coupled to at least one suture or a plurality of the elongated members, configured to deflect the elongated members by tensioning or slacking the sutures, and wherein the deflection of the elongated member can be configured to various positions that allow for repeated stabilizing, grasping and/or release of the native leaflets.

268. The device of clauses of 1 and 2, wherein, the elongate members are made of elastic, super elastic, shape memory, nitinol, metals, alloys, plastics, and/or ceramics.

269. The device of clauses of 1 and 2, wherein, the atrial side elongate members are designed to atraumatically grasp or release the native leaflet.

270. The device of clauses of 1 and 2, wherein, at least some elongate members comprise of atraumatic barbs.

271. The device of clauses of 1 and 2, wherein, the ventricular side elongate members are designed to atraumatically grasp or release the native leaflet.

272. The device of clauses of 1 and 2, wherein, at least one elongate member is covered with fabric, mesh, coating and/or surface features that allow or promote tissue encapsulation.

273. The device of clauses of 1 and 2, wherein, at least one elongate member is releasably attached to the suture.

274. The device of clauses of 1 and 2, wherein, at least one suture is configured to lift the native leaflet off the elongate members.

275. The device of clauses of 1 and 2, wherein, at least one suture is configured with an inverter.

276. The device of clauses 1 and 2, wherein, the elongate members are a pair of inner and outer arms at the ventricular side and/or the elongate members are grippers in the atrial side as in previous co-owned patent applications US20200383782A1, PCT/US2017/042003 and/or PCT/0S2019/013853.

277. A capture device for fixation of leaflets of a cardiac valve comprising:
at least one distal element adapted to be extend radially outward from a center of the capture device after the capture device is advanced to a location near the cardiac valve of the heart, the at least one distal element being configured to be atraumatically positioned against at least one leaflet of the cardiac valve; wherein at least one distal element has a bias configured to extend outward and optionally invert from the center of the capture device when the suture is tensioned towards a tensioned condition to enable release of the at least one leaflet previously captured between the at least one proximal element and the at least one distal element.
at least one proximal element held proximally upward by a suture in a tensioned condition, wherein the at least one proximal element has a bias configured to extend radially outward from the center of the capture device when the suture is slacked towards a slacked condition to enable capture of the at least one leaflet between the at least one proximal element and the at least one distal element.

278. A capture device for fixation of leaflets of a cardiac valve comprising:
at least one distal element held distally in an extended outward or inverted configuration by a suture in a tensioned condition, wherein the at least one distal element has a bias configured to shrink radially inward towards the center of the capture device when the suture is slacked towards a slacked condition to enable capture of the at least one leaflet between the at least one proximal element and the at least one distal element.
at least one proximal element held proximally upward by a suture in a tensioned condition, wherein the at least one proximal element has a bias configured to extend radially outward from the center of the capture device when the suture is slacked towards a slacked condition to enable capture of the at least one leaflet between the at least one proximal element and the at least one distal element,

279. The device of clauses of 11 and 12, wherein, the outward bias of the proximal element is less resilient than the inward bias of the distal element.

280. The device of clauses 11 and 12, wherein the at least one distal element includes a pair of distal elements, and the at least one proximal element includes a pair of proximal elements.

281. The device of clauses of 11 and 12, wherein the at least one distal element comprises a loop.

282. The device of clauses of 11 and 12, wherein the at least one distal element comprises a wire.

283. The device of clauses of 11 and 12, wherein the at least one distal element comprises a petal shape.

284. The device of clauses of 11 and 12, wherein the at least one proximal element is biased toward the at least one distal element.

285. The device of clauses of 11 and 12, wherein at least one proximal element comprises nitinol.

286. The device of clauses of 11 and 12, wherein the at least one proximal element comprises at least one friction accessory extending therefrom.

287. The device of clauses of 11 and 12, wherein the at least one friction accessory comprises at least one barb.

288. The device of clauses of 11 and 12, wherein the at least one friction accessory comprises a plurality of barbs.

289. The device of clauses of 11 and 12, wherein the at least one friction accessory comprises a tissue penetration depth limiting feature in the barb.

290. The device of clauses of 11 and 12, wherein the at least one friction accessory comprises a tissue penetration depth limiting feature in the barb.

291. The device of clauses of 11 and 12, wherein the at least one proximal element is shorter in length to the distal element.

292. The device of clauses of 11 and 12, wherein the at least one proximal element is longer in length to the distal element.

293. The device of clauses of 11 and 12, wherein the at least one proximal element is equal in length to the distal element.

294. The device of clauses of 23, 24, and/or FIGS. 23, 24, and 25, wherein the at least one proximal element is 0, 0.1, 0.3, 0.6, 0.9, 1.2, 1.5, 1.8, 2.1, 2.4, 5, 10, 20, 30 and/or 100 mm more than the length of the proximal elements.

295. The device of clauses of 23, 24, and/or FIGS. 23, 24, and 25, wherein the at least one proximal element is 0, 0.1, 0.3, 0.6, 0.9, 1.2, 1.5, 1.8, 2.1, 2.4, 5, 10, 20, 30 and/or 100 mm less than the length of the proximal elements.

296. The device of clauses of 11 and 12, wherein the at least one pair of proximal and distal element is configured to capture leaflet is 0, 0.1, 0.3, 0.6, 0.9, 1.2, 1.5, 1.8, 2.1, 2.4, 5, 10, 20, 30 and/or 100 mm longer than the length of the next pair of the proximal and distal elements.

297. The device of clauses of 11 and 12, wherein the at least one pair of proximal and distal element is configured to capture leaflet and the bias force of the proximal element is significantly less than the next pair of the proximal and distal elements.

298. The device of clauses of 11 and 12, wherein the at least one pair of proximal and distal element is configured to capture leaflet and the bias force of the proximal element is significantly more than the next pair of the proximal and distal elements.

299. The device of clauses of 11 and 12, wherein the at least one pair of proximal and distal element is configured to capture leaflet and the bias force of the proximal element is about the same as the next pair of the proximal and distal elements.

300. The device of clauses of 11, 12, and/or FIGS. 23, 24, and 25, wherein the distal elements are configured with bias force greater than the proximal elements.

301. The device of clauses of 11, 12, and/or FIGS. 23, 24, and 25, wherein the distal elements are configured with bias force that is 0, 0.14, 0.3, 0.6, 0.9, 1.2, 1.5, 1.8, 2.1, 2.4, 5, 10, 20, 30 and/or 100 lbf more than the bias force of the proximal elements.

302. The device of clauses of 11, 12, and/or FIGS. 23, 24, and 25, wherein the distal elements are configured with bias force that is 0, 0.14, 0.3, 0.6, 0.9, 1.2, 1.5, 1.8, 2.1, 2.4, 5, 10, 20, 30 and/or 100 lbf less than the bias force of the proximal elements.

303. An endovascular heart valve repair system comprising:
a delivery catheter having a distal end configured to be introduced into a heart chamber adjacent to a pair of coaptating heart valve leaflets, said delivery catheter including a release bar having a pair of inverters;
a valve repair leaflet grasping device comprising a hub configured to be removably attached to the release bar of the delivery catheter, a first pair of leaflet capture arms comprising of a inner arm and a outer arm coupled to the hub, and a second pair of leaflet capture arms comprising a second inner arm and a second outer arm coupled to the hub; and
a first set of control tethers positioned on or through the delivery catheter and coupled to the outer arms and configured to selectively bias the outer arms into a valve leaflet capture position; and
a second set of tethers positioned on or through the delivery catheter and coupled to the inner arms and configured to selectively bias the inner arms into a valve leaflet capture position;
wherein the first set of control tethers are threaded through laterally spaced-apart locations on the inverters so that drawing proximal portions of the of the first set of control tethers in a proximal direction causes distal portions of first set of control tethers to pull outer segments of the outer arms in a distal direction into the valve leaflet capture, stabilization, or release position.
wherein the second set of control tethers are threaded through laterally spaced-apart locations on the inverters so that drawing proximal portions of the of the second set of control tethers in a proximal direction causes distal portions of first set of control tethers to pull outer segments of the outer arms in a distal direction into the valve leaflet capture, stabilization, or release position.

304. An endovascular heart valve repair system comprising:
a delivery catheter having a distal end configured to be introduced into a heart chamber adjacent to a pair of coapting heart valve leaflets, said delivery catheter including a release bar having at least one inverter;
a valve repair leaflet grasping device comprising a hub configured to be removably attached to the release bar of the delivery catheter, at least one pair of leaflet capture arms comprising of an inner arm and an outer arm coupled to the hub, and;
at least one pair of control tethers, wherein the first tether is positioned on or through the delivery catheter and coupled to the outer arms and configured to selectively bias the outer arms into a valve leaflet capture position; and the second tether is positioned on or through the delivery catheter and coupled to the inner arms and configured to selectively bias the inner arms into a valve leaflet capture position;
wherein at least one pair of the control tethers are threaded through laterally spaced-apart locations on the inverters so that drawing proximal portions of the control tethers in a proximal direction causes distal portions of the control tethers to pull outer segments of the outer arms in a distal direction into the valve leaflet capture, stabilization, or release position.
at least one set of control tethers are threaded through the laterally spaced apart locations on the inverters and the Release Bar and/or through the device, so that drawing proximal portions of the control tethers causes the tether to aid bail-out by releasing and or removal of any leaflet captured between the gap of the inner and outer arms in the leaflet release configuration.

305. An endovascular heart valve repair device as in the clauses 36 and 37, comprising of an expandable spacer, configured to prevent back flow of blood/fluid.

306. An endovascular heart valve repair device as in clauses 36 and 37, wherein the pair of inverters comprises a first inverter extending laterally in a first direction from a distal tip of the delivery catheter and a second inverter extending laterally in a second direction from a distal tip of the delivery catheter.

307. An endovascular heart valve repair device as in clauses 36 and 37, wherein the first and second directions are opposite to each other.

308. An endovascular heart valve repair device as in clauses 36 and 37, wherein each of the first and second inverters is pivotally attached to the distal tip of the delivery catheter.

309. An endovascular heart valve repair device as in clauses 36 and 37, wherein the pivotal attachment is configured so that the inverters laterally deploy when the first tethers are pulled proximally to apply an opening force to the inverters but are able to axially collapse in alignment with the delivery catheter in the absence of the opening force.

310. An endovascular heart valve repair device as in clauses 36 and 37, wherein the first set of tethers pass from a distal end of the release bar, are slidably coupled to each of the inverters and the outer arms and are fixedly attached to the release bar.

311. An endovascular heart valve repair device as in clauses 36 and 37, wherein the second set of tethers pass from a distal end of the delivery catheter, are slidably coupled to each of the inner arms, and are fixedly attached to the release bar.

312. An endovascular heart valve repair device as in clauses 36 and 37, wherein the inner and outer arms comprise inner and outer leaf springs.

313. An endovascular heart valve repair device as in clauses 36 and 37, wherein the inner leaf springs are biased to open laterally outwardly away from the release bar and the outer leaf springs are biased to close laterally inwardly toward the release bar so that the leaflets may be captures therebetween when the leaf springs are unbiased.

314. An endovascular heart valve repair device as in clauses 36 and 37, wherein the outward opening bias of the inner leaf springs is less than inward closing bias of the outer leaf springs.

315. An endovascular heart valve repair device as in clauses 36 and 37, wherein the outer leaf springs are generally straight and lie closely over the release bar when unbiased so that the outer leaf springs will laterally close the inner leaf springs when all leaf springs are free from bias.

316. A valve repair leaflet grasping device comprising: a hub removably attached to a deployment shaft; two pairs of outer and inner arms configured to be biased apart to create a leaflet capture space; a spacer expandable member typically an inflatable balloon or a mesh to act as a spacer; an inverter to ungrasp the incorrectly grasped leaflets; barbs in the inner arms for no-slip grasp; a detachable tether to inflate or deflate the said spacer.

317. A leaflet grasping device as in clause 49, wherein said inverter ungrasps the said distal arms from the leaflets and repositions the said distal arms. Active movement of the said arms in the left ventricle achieved with the help of the said ventricular sutures and the said inverter after the said arms have been deployed and grasped the leaflets.

318. An embodiment of clause 49, further comprising of grippers that can be used in the atrial side along with the arms in the ventricular side to facilitate better positioning, engaging, repositioning, and manipulating the leaflets from the top and the bottom planes of the heart valve.

319. A leaflet grasping device as in clause 49, wherein, said atrial suture when manipulated ungrasps the arm from the leaflets without an inverter.

320. A valve repair leaflet grasping device as in clause 49, wherein said spacer engages one leaflet while the one set of the said inner arms and said outer arms engage the other leaflet.

321. A valve repair leaflet grasping device, wherein said expandable member or balloon has lower compliance and is rigid to engage one leaflet while the other leaflet is engaged by one set of the said inner arms and said outer arms.

322. A method for the valve repair leaflet grasping device as in clause 49, wherein said expandable member or balloon has higher compliance making the said expandable member to conform to one leaflet based on the pressure exerted on the said expandable member while the other leaflet is engaged by one set of the said inner arms and said outer arms.

323. A method for the valve repair leaflet grasping device as in clause 49, wherein said detachable tether tube as in US20180185185 obalon can be employed to adjust the said expandable member to the size and shape of the space during the procedure and post implantation.

324. A valve repair leaflet grasping device as in clause 49, wherein said barbs are attached to the said inner arms to enable a tight non-slip grip of the leaflets once they are grasped.

325. A valve repair leaflet grasping device as in clause 49, wherein said pair of inner arms are longer than the outer arms with a less than 1 mm the distance between the leaflets after they are grasped.

326. A valve repair leaflet grasping device as in clause 49, wherein said pair of inner arms are flushed with outer arms with a less than 1 mm the distance between the leaflets after they are grasped. The said inner arms and the said outer arms are of the same height.

327. A valve repair leaflet grasping device as in clause 49 comprising: a pair of inner arms shorter than the outer arms. This allows the leaflets to touch each other forming tissue in-between as there is no space in-between the leaflets.

328. An expandable member, further comprising a funnel-shaped expandable mesh that elastically and resiliently takes the shape of a funnel when pushed out of the guide catheter to encompass the implant either partially or fully, retracts the implant back into the guide catheter and takes the shape a tube and fits perfectly inside the delivery catheter.

329. A method for clause 49 as in FIG. 31, wherein the distance indicated by 315 is between 0, 1, 2, 3, 4, . . . , 24, and/or 25 cm and the distance indicated by 313 is from 0, 1, 2, 3, . . . , 99 and/or 100 cm.

330. A retractor device comprising: a member that captures a previously deployed implant; a steerable guide catheter; a delivery catheter

331. A feature of clause 64, wherein the said member is a coiled leaf spring that uncoils when pushed, captures the implant and encompasses the implant. The said member is encapsulated by the guide catheter to bail out the implant with a single arm.

332. A feature of clause 64, where the said member has two coiled leaf spring that uncoils when pushed, captures the implant and is encompassed by the said guide catheter to bail out implant with two arms.

333. A feature of clause 64 where the said member is a fan shaped structure that captures the implant and is encompassed by the said guide catheter to bail out certain implants.

334. An embodiment of clause 64, further comprising a spring coil made of laser cut nitinol tube encapsulating the said delivery catheter to enable easy maneuver at tight curves. The said spring coil also positions the said catheter in a straight manner at all times.

335. A rescue catheter comprising: a shaft; a distal end with a slit for retrieval of the said delivery catheter and the implant.

336. A method of clause 69, wherein the said rescue catheter captures and completely encompasses the implant along with the delivery catheter and enables bail out of the implant via the said guide catheter.

337. A method of clause 69, wherein the said rescue catheter is inserted over the guide catheter.

338. A implant capture rescue catheter comprising:

339. An implant delivery catheter, and;

340. a guide catheter,

341. wherein, the rescue catheter has a cylindrical cross-section with a slit and/or a ‘c’ shaped cross-section, and a long shaft that runs the length of the delivery catheter and/or the guide catheter, extending proximally.

342. A rescue catheter of clause 72, wherein, the rescue catheter is configured to be inserted into the guide catheter but over the deliver catheter.

343. A rescue catheter of clause 72, wherein, the cylindrical section is expandable, and configured to expand when extended beyond the distal tip of the guide catheter, and help retract the implant delivery catheter into the guide catheter, when retracted.

344. A rescue catheter of clause 72, wherein, the cylindrical section is configured to slide over the guide catheter, atraumatically.

345. A device described as in example FIG. 42, wherein, the thickness of the OuterArms is preferably about 0.33 mm.

346. A device described as in example FIG. 42, wherein, the thickness of the OuterArms is 0.12, 0.16, 0.20, . . . , 3.12, and/or 3.15 mm.

347. A device described as in example FIG. 42 and/or FIG. 46-50, wherein, the thickness of the Grippers is preferably about 0.20 mm.

348. A device described as in example FIG. 42 and/or FIG. 46-50, wherein, the thickness of the Grippers is between 0.12, 0.16, 0.20, . . . , 3.12, and/or 3.15 mm.

349. A device described as in example FIG. 42 and/or FIG. 46-50, wherein, the cross-section at bend region of the OuterArms is greater than Gripper bend cross-section by 0, 1, 2, 3, . . . , 999, and/or 1000%.

350. A device described as in example FIG. 42 and/or FIG. 50, wherein, the FCoapt is between 0, 0.1, 0.2, 0.3, . . . , 19.9, and/or 201bf.

351. A device described as in example FIG. 42 and/or FIG. 50, wherein, the distance between the leaflets at the tip of the device is about or in between 0, 0.1, 0.2, 0.3, . . . , 4.9, and/or 5.0 mm, preferably less than 1 mm.

352. A device described as in example FIG. 42 and/or FIG. 50, wherein, the Arms and/or Grippers may be configured to have a self-biasing strain of about 0, 0.1, 0.2, 0.3, . . . , 19.9, and/or 20%, preferably between 1% and 6%.

353. A device described as in example FIG. 42 and/or FIG. 50, wherein, the Outer Arm is bent at the tip, as in FIG. 49, in a configuration that maximizes leaflet coaptation.

### BRIEF DESCRIPTION OF THE DRAWINGS

FIG. 1A shows an exemplary embodiment of an outer arm component of a tissue fixation device with a compressible spring based single ring and/or loop with a slit comprised of a compressive wire wherein the proximal portion of the ring is fixed onto the outer arm.

FIG. 1B shows the same outer arm with a continuous ring and/or loop wherein the proximal portion of the ring is fixed onto the outer arms at one or two and or more ends.

FIG. 1C shows the same arm with a continuous ring and/or loop with a fixed proximal portion and a freestanding distal portion. The ring may be circular, oval, rectangular, z, s, v, u, w, zigzag and or any compressible shape.

FIG. 2 shows an exemplary embodiment of an outer arm comprised of expandable tines.

FIG. 3 shows an exemplary embodiment of an outer arm comprised of a expandable stent material that shortens in length as it radially and/or laterally expands.

FIG. 4A shows an exemplary embodiment of an outer arm comprised of an elongated slot of compound geometry.

FIG. 4B shows the outer arm as it expands laterally to a diamond-shaped configuration.

FIG. 5A shows an exemplary embodiment of a split outer arm.

FIG. 5B shows the outer arm in FIG. 5A expanded laterally to a V-shaped configuration.

FIG. 6A shows an exemplary embodiment of an outer arm comprising of expandable stent patterns.

FIG. 6B illustrates an exemplary expandable wire profile that can be attached to the outer arm.

FIG. 7A illustrates the side view of an exemplary embodiment of a tissue fixation device wherein the outer arm component of the tissue fixation device expands in a folding (Japanese) fan-like design.

FIG. 7B shows an alternative view of the said 7A device.

FIG. 8A shows a top-down view of the proximal portion of the ReleaseBar comprising of a plurality of small openings 60 for actuation suture/wire and a plurality of smaller openings 62 for the guidewire/release mandrel.

FIG. 8B shows the release bar with a plurality of openings for anchoring suture loops.

FIG. 8C shows the openings 78 on the release bar that can be used to configure the suture loops.

FIG. 8D shows exemplary bailout suture 93 looped through the openings shown in FIG. 8C.

FIG. 9A shows a schematic of a bailout suture as part of the delivery catheter wherein the bailout suture is not deployed.

FIG. 9B illustrates the schematic in FIG. 9A, wherein, the bailout suture is deployed.

FIGS. 10A and 10B shows an alternative configurations of the bailout suture using additional restraining suture loops 120, 122.

FIGS. 11A and 11B illustrate an exemplary embodiment with automatic bailout feature as a part of the implant.

FIGS. 12A to 12D illustrate an exemplary embodiment comprising of manually actuatable bailout suture that is part of the implant and interacts with both outer arms and inner arms.

FIGS. 12E to 12G illustrate an exemplary embodiment comprising of manually actuatable bailout suture that is part of the implant and interacts with outer arms.

FIGS. 13A to 13C illustrate an exemplary method to using nitinol (or shape memory or superelastic materials) based motors and actuators.

FIGS. 14A and 14B illustrate an exemplary embodiment of a tissue fixation device, wherein, the outer arm is electrically actuated via a nitinol wire, strip, sheet metal, and/or suture.

FIGS. 15A and 15B illustrate an alternate embodiment of a device constructed in accordance with the principles of the present invention.

FIGS. 16A to 16C show exemplary schematics of an electrically actuated steerable catheter, using various configurations of actuator wire within a catheter shaft to steer it.

FIG. 17A illustrates a mitral valve fixed in a double orifice using an exemplary embodiment of a device with expanding balloons. The balloons may be variably expanded either during the procedure or remotely adjusted post procedure.

FIG. 17B shows an exemplary embodiment of a device with dynamically (volume adjusting based on patient activity or heart rate or other vital signs such as blood pressure) expanding balloons fixed onto mitral valves with anatomical defects. One or more balloons maybe used. Balloons maybe remotely tethered/inflated. Variable balloon attachment positions to the implant with varying number of balloons may be configured based on disease or target mitral valve (or for tricuspid valve).

FIGS. 18-21 show large gap between the opposing leaflets in a MitraClip® device.

FIG. 22 shows x-ray image of an exemplary Straight device 255 post implantations.

FIG. 23 shows that the Straight device 255 has smooth and continuous tissue bridge 257 post implantation.

FIG. 24 Curved device 260 and its small gap 263

FIGS. 25 and 26 show an exemplary device with large gap filled with spacer between apposing leaflets filled with a spacer.

FIGS. 27-30 show various embodiments of supports and spacers to fill gap between apposing leaflets.

FIGS. 31-37 show various embodiments of supports and spacers to fill gap between apposing leaflets in an exemplary device.

FIGS. 38-41 show an exemplary device previously described in co-owned patent application US20200383782A1. These figures highlight the leaf spring self-bias and coapting/cinching forces, when assembled.

FIGS. 42A to 44C show various exemplary embodiments depicting size variations of the inner arms vs. the outer arms at the tip of the device.

FIG. 45 shows schematic of a normal mitral valve in systole.

FIG. 46 shows an exemplary schematic of a mitral valve with a gap in systole to represent MR.

FIG. 47 shows a larger annular distance with exemplary v-shaped device

FIG. 48 shows how the large annular distance causing poor cinching and resulting in residual gaps, thereby causing suboptimal reduction in MR.

FIG. 49 shows an exemplary device that coapts and cinches the annulus better than v-shaped device in FIG. 47.

FIG. 50 shows superior cinching results in optimal reduction in MR.

FIG. 51 shows an exemplary schematic of a mitral valve with a very large gap in systole to represent severe MR.

FIG. 52 shows reduction in MR with an exemplary tissue coapting device.

FIG. 53 show reduction in MR with an exemplary tissue coapting device with side extending spacers.

FIGS. 54-65 show alternative embodiments to improve coaptation and cinching of leaflets.

FIGS. 66A and 66B show an exemplary embodiment of a tissue grasping device with outer arms comprised of different-sized leaf springs with multiple layers for additional mechanical strength.

FIGS. 67A and 67B illustrates an exemplary embodiment of an outer arm with inner arm as part of the said outer arm.

FIGS. 68A and 68B illustrates a schematic of an exemplary embodiment of a tissue fixation device wherein inner arm and outer arm are configured as one piece.

FIGS. 69A and 69B illustrate an exemplary embodiment of a tissue fixation device with a spring biased outer arm, actuated using wires or sutures.

FIGS. 70A to 70C illustrate an exemplary embodiment of a tissue fixation device comprising of discrete inner arms attached to the outer arm, based on common umbrella design.

FIG. 71A shows a top-view schematic of an exemplary embodiment of a tissue fixation device configured from coaxial metal tubes.

FIG. 71B shows a side view of the same device.

FIG. 71C shows an alternative configuration of the device as shown in FIG. 71B with the outer arms biased outward.

FIGS. 72-76 show an exemplary embodiment of alternate self-closing, self-biased valve repair system.

FIG. 77 shows an exemplary isometric view of the griper of the MitraClip® device.

FIG. 78 shows the Arm angle at the base and at the tip of an exemplary embodiment.

FIG. 79 The distance between the arms at the tip and the widest distance between the arms of an exemplary embodiment.

FIG. 80 Current MitraClip® Barb design with a sharp point 523

FIGS. 81 to 83 show various embodiments of exemplary blunted or rounded barb points.

FIG. 84 illustrates an exemplary device with an expandable stent, mesh, and or balloon acting as a spacer positioned in-between the leaflets.

FIG. 85 illustrates an alternate configuration with three separate inflatable or self-expandable stent, mesh, and or balloon acting as a spacer positioned in-between the leaflets designed to fill the gap in between the leaflets completely (referenced in FIG. 87, PCT/0S2019/013853).

FIG. 86 An exemplary delivery catheter interface, as explained in co-owned U.S. Application No. US20200383782A1, with one inverter attached with the Release Bar.

FIG. 87 shows an exemplary embodiment of an implant mounted on the delivery system shown in FIG. 86.

FIG. 88 Exemplary embodiment of an implant with a single pair of Gripper and Outer Arm.

FIG. 89 Outer arm of the implant biased toward a grasping angle.

FIGS. 90 to 97 show exemplary embodiments of a single pair of Gripper and Arm, with various configurations of expandable spacers.

FIG. 97 illustrates the anatomical cross-section of a heart showing right atrium (RA) and right ventricle (RV).

FIG. 98 illustrates the heart in FIG. 97 with a catheter inserted through the inferior vena cave (IVC) via the femoral vein access. Note the sharp U-turn the catheter needs to make to reach the tricuspid valve.

FIG. 99 illustrates the heart in FIG. 97 with a catheter inserted through the superior vena cava (SVC) via the jugular vein access.

FIGS. 100 and 101 shows an exemplary embodiment of ReleaseBar with the center posts.

FIGS. 102 to 108 show an exemplary embodiment of a mitral valve replacement device with actuatable Gripper and or Arms for superior anchoring.

FIG. 109A shows exemplary steerable guide catheter mounted on a Stabilizer.

FIG. 109 B shows a device delivery catheter co-mounted along with steerable guide and stabilizer shown in FIG. 28A.

FIG. 109 C shows exemplary distal section of the steerable guide shaft and device delivery catheter.

FIG. 109 D shows exemplary device delivery catheter with flush port (and or manual bailout actuation rod).

FIG. 110 shows an exemplary steerable guide catheter design incorporating stiffeners.

FIGS. 111 to 119D show exemplary delivery system of the tissue grasping device.

FIGS. 120 to 125 show various exemplary embodiment configurations of the delivery system along with the implant and a funnel shaped mesh to retract the implant into the delivery catheter.

FIGS. 126A to 126D show exemplary embodiments with retractable steel or plastic coil features over the delivery catheter to help retract the device into the delivery catheter.

FIGS. 127A and 127B show exemplary embodiments comprising of retractable fan or cone shaped the features to help retract the device into the delivery catheter. A delivery catheter comprising of an external spring 625 in the distal segment, configured to reduce friction and maintain straightness when it is advanced out of the guide catheter, as shown in FIG. 128.

FIGS. 129A and 129B show an exemplary embodiment of rescue catheter with a slit, which aid in retraction of the device into the guide catheter. FIGS. 129C and 129D show rescue catheter having a slit 670 on a proximal portion of its shaft 650.

FIG. 130A shows cross sectional view of the balloon spacer placed in between two inner arms of the device in between the leaflets.

FIG. 130B shows an alternate cross-sectional view of the balloon placed in between two inner arms. An adjustable spacer balloon can be inflated and or deflated after grasping the leaflets.

The following is a listing of the reference numbers used in this application:

| IVC | Inferior vena cava |
|---|---|
| LA | Left atrium |
| LF | Heart valve leaflet |
| LV | Left ventricle |
| MV | Mitral valve |
| PA | Pulmonary artery |
| PM | Papillary muscle of the left ventricle |
| PV | Pulmonary valve |
| RA | Right atrium |
| RV | Right ventricle |
| SVC | Superior vena cava |
| TV | Tricuspid valve |
| 1 | Exemplary embodiment of an outer arm |
| 3 | Attachment feature such as a screw, weld, glue, and or suture |
| 5 | Single ring/loop made of compressive wire |
| 7 | Continuous, single ring/loop made of compressive wire |
| 9 | Continuous, circular wire with a fixed proximal portion and a freestanding distal portion |
| 11 | Exemplary embodiment of an outer arm |
| 13 | Expandable segment of outer arm 11 |
| 15 | Expandable segment of outer arm 11 |
| 17 | Direction of feature 19 as it is pulled to grasping position |
| 19 | Segment of outer arm 11 |
| 21 | Exemplary embodiment of an outer arm comprising of a closed cell slit |
| 23 | Radial and/or lateral expansion of outer arm 21 |
| 25 | Shortening of outer arm 21 as said arm is expanded |
| 27 | Radial and/or lateral expansion of outer arm 21 |
| 30 | Exemplary embodiment of an outer arm comprising of a closed cell slit |
| 32 | Left expansion of outer arm 30 |
| 34 | Right expansion of outer arm 30 |
| 36 | Exemplary embodiment of an outer arm comprising of an open cell slit |
| 38 | Left expansion of outer arm 36 |
| 40 | Right expansion of outer arm 36 |
| 42 | Exemplary embodiment of an outer arm with stent like laser cut patterns |
| 44 | Exemplary expanded stent pattern |
| 46 | Exemplary embodiment of an outer arm |
| 48 | Compressible/expandable feature comprising of a wire |
| 50 | Left outer base |
| 52 | Folding fan-like inner arm |
| 54 | Right outer base |
| 56 | Screw |
| 58 | Screw |
| 60 | Opening in proximal portion of release bar for insertion of actuation suture/wire |
| 62 | Opening in proximal portion of release bar for insertion of guidewire/release mandrel |
| 64 | Proximal portion of release bar 86 |
| 66 | Tenth opening on release bar 86 |
| 68 | Ninth opening on release bar 86 |
| 70 | Eighth opening on release bar 86 |
| 72 | Seventh opening on release bar 86 |
| 74 | Sixth opening on release bar 86 |
| 76 | Fifth opening on release bar 86 |
| 78 | Fourth opening on release bar 86 |
| 80 | Third opening on release bar 86 |
| 82 | Second opening on release bar 86 |
| 84 | First opening on release bar 86 |
| 86 | Release bar |
| 88 | Release rod |
| 91 | Direction of cinch suture loop 99 as it is retracted to cinch bailout |
| | suture 118 segments 97, 106, 95, 102 and allow leaflet capture |
| 93 | Segment of cinch suture 99 |
| 95 | Segment of bailout suture |
| 97 | Segment of bailout suture |
| 99 | Cinch suture loop, used to cinch Bailout suture 118 |
| 100 | Segment of cinch suture loop 99 |
| 102 | Segment of cinch suture loop 99 |
| 104 | Segment of bailout suture 95 |
| 106 | Segment of bailout suture 97 |
| 108 | Suture loop around inverter 110 |
| 110 | Left inverter |
| 112 | Right inverter |
| 114 | Suture loop around inverter 112 |
| 116 | Segment of bailout suture 118 |
| 118 | Bailout suture |
| 120 | Left suture loop around opening 84 on release bar 86 |
| 122 | Right suture loop around opening 84 on release bar 86 |
| 124 | Segment of bailout suture 118 |
| 126 | Direction of bailout suture 118 as it is retracted to expunge |
| | caught tissue and/or chordae during leaflet capture |
| 128 | Schematic representation of the left Inner arm |
| 130 | Schematic representation of the right Inner arm |
| 132 | Automatic bailout suture looped through inner arm 128 and outer arm 136 |
| 134 | Automatic bailout suture looped through inner arm 130 and outer arm 138 |
| 136 | Schematic representation of the left Outer arm |
| 138 | Schematic representation of the right Outer arm |
| 144 | Direction of outer arm 138 to inverted outer arm 140 |
| 146 | Bailout actuation suture that pulls bailout suture taut when retracted |
| 148 | Segment of bailout suture between inner arms 128, 130 |
| 154 | Left bailout suture segment |
| 156 | Right bailout suture segment |
| 162 | Direction of inner arm 128 to capture leaflet |
| 164 | Direction of inner arm 130 to capture leaflet |
| 166 | Direction of retraction of actuation suture 146 |
| 173 | Left outer arm |
| 175 | Right outer arm |
| 177 | Left inner arm |
| 179 | Right inner arm |
| 181 | Electrically actuated straight wire and/or suture |
| 182 | Suture or connecting wire |
| 183 | Base with a rod |
| 185 | Electrically actuated coil and/or spring |
| 187 | Left Nitinol leafspring actuator |
| 189 | Right Nitinol leafspring actuator |
| 191 | Movement of left Nitinol actuator motor |
| 195 | Actuator wire |
| 197 | Steerable catheter shaft |
| 199 | Percent of stroke |
| 201 | Actuator wire |
| 203 | Actuator wire |
| 205 | Actuator wire |
| 207 | Right steering direction of catheter 209 after actuation |
| 209 | Electrically steerable catheter |
| 211 | Actuator wire |
| 213 | Actuator wire |
| 215 | Actuator wire |
| 217 | Actuator wire |
| 219 | Left steering direction of catheter 209 after actuation |
| 221 | Down steering direction of catheter 209 after actuation |
| 223 | Actuator wire |
| 225 | Up steering direction of catheter 209 after actuation |
| 227 | Actuator wire |
| 229 | First inner arm of an exemplary embodiment of a clip |
| 231 | Second inner arm of an exemplary embodiment of a clip |
| 235 | First inflatable, static balloon attached to the clip's base |
| 237 | Second inflatable, static balloon attached to the clip's base |
| 239 | First inflatable, dynamic balloon attached to the clip's base |
| 241 | Second inflatable, dynamic balloon attached to the clip's base |
| 243 | Motor, Pump, Actuator and/or sensor |
| 245 | Motor, Pump, Actuator and/or sensor |
| 247 | Bare metal components in the pocket of MitraClip ® device 251 |
| 249 | Gap between the MitraClip ® arms 250 |
| 250 | MitraClip ® arms |
| 251 | Exemplary MitraClip ® device |
| 253 | Blood clot in MitraClip ® device |
| 255 | X-ray image of an exemplary embodiment of the device as implanted |
| 257 | Exemplary animal study data showing smooth and pocketless |
| | tissue bridge post 180 days using an implant 255 |
| 260 | Exemplary curved implant |
| 262 | Exemplary grippers of curved device 260 |
| 263 | Pocket in the exemplary curved implant 260 |
| 264 | An exemplary spacer that is attached either one or both Grippers 262 |
| 266 | Exemplary arms of curved device 260 |
| 268 | An exemplary spacer in the atrial side between grippers |
| 270 | An exemplary spacer that are attached to each individual Gripper 262 |
| 272 | An exemplary expendable wire form spacer in the ventricular side attached to arms |
| 274 | An exemplary spacer that is attached to ventricle side of the arm |
| 276 | An exemplary spacer that is attached to atrial side of the arm |
| 278 | Suture, glue, weld, and or fastener attaching the spacer 264 to the Gripper 287 |
| 280 | A schematic picture of MitraClip ® device |
| 282 | Arm of the MitraClip ® implant |
| 287 | Gripper of MitraClip ® implant |
| 290 | The Edwards Pascal implant |
| 300 | Configuration of the implant with the inner arm 309 and outer arm 307 of the same height |
| 305 | Medially placed barbs on the Arm |
| 307 | Outer Arm |
| 309 | Inner Arm |
| 311 | Outer Base |
| 315 | Slot in the inner arm |
| 317 | Hole through which screws go in |
| 319 | Hole through which screws go in |
| 321 | Slot in the outer arm |
| 325 | Configuration of the implant with the inner arm 309 longer than outer arm 307 |
| 330 | Configuration of the implant with the inner arm 309 shorter than outer arm 307 |
| 335 | Schematic of the normal mitral valve in systole |
| 340 | Schematic of the mitral valve with MR in systole |
| 342 | Gap causing MR in native mitral valve in systole |
| 344 | Schematic of exemplary implant in V-shaped coaptation |
| 346 | Diametric distance between annulus for v-shaped implant 344 |
| 348 | The gap between leaflets in systole after an implant is deployed |
| 350 | Schematic of exemplary implant in coaptation |
| 352 | Exemplary laterally expandable spacers |
| 354 | Diametric distance between annulus for implant |
| 360 | Arm of exemplary implant |
| 362 | Flexible or bendable arms of an exemplary tissue grasping |
| | implant that may be superelastic or with a spring hinge |
| 364 | The removable segment of actuation rod, used to actuate the arm 360, 362, 366 |
| 366 | Arm of the MitraClip ® implant with an extra pivot or hinged portion. |
| 367 | Lever from the tip of the Arm to the point of contact with spring 370 |
| 368 | The implantable segment of actuation rod, used to actuate the arm 360, 362, 366 |
| 369 | Lever from the hinge of the Arm to the point of contact with spring 370 |
| 370 | Spring mechanism that may be superelastic |
| 371 | Lever from the hinge of the Arm to the tip of the spring 370 |
| 372 | A hinge in the arm 366 that may comprise of a spring |
| 374 | Discrete leaf spring that provides required biasing forces to coapt arms 360, 362, 366 |
| 380 | Outer base |
| 382 | Outer arm of length B |
| 384 | Outer arm of length A |
| 386 | Inner arm |
| 388 | Inner arm |
| 390 | Outer arm of length C |
| 392 | Outer arm of length D |
| 394 | Direction of outer arms 384, 382 |
| 396 | Inner arm |
| 398 | Barb |
| 400 | Outer Arm |
| 402 | Outer arm 394 in open position |
| 404 | Inner arm |
| 408 | Outer arm |
| 410 | Movement of outer arm 394 to open position |
| 412 | Inner arm |
| 414 | Outer arm |
| 416 | Eyelet of outer arm 414 allowing suture 418 to base 420 |
| 418 | Suture connecting outer arm 414 to spring-loaded base 420 |
| 420 | Spring-loaded outer base |
| 422 | Suture connecting outer arm 428 to spring-loaded base 420 |
| 424 | Eyelet of outer arm 414 allowing suture 422 to base 420 |
| 426 | Inner arm |
| 428 | Outer arm |
| 430 | Leaflet release rod |
| 433 | Retraction of leaflet release rod 430 to raise outer arms 414, 428 to release LF |
| 435 | Actuation suture and/or wire and/or mandrel |
| 437 | Delivery catheter tube |
| 439 | Left inner arm attached to outer arm 443 or alternatively, part of the outer arm 443 |
| 441 | Right inner arm attached to outer arm 445 or alternatively, part of the outer arm 445 |
| 443 | Left segment of an exemplary outer arm made of a continuous sheet metal and/or strip metal |
| 445 | Right segment of an exemplary outer arm made of a continuous sheet metal and/or strip metal |
| 447 | Left rigid or flexible feature used to actuate outer arms 443, 445 |
| 449 | Right rigid or flexible feature used to actuate outer arms 443, 445 |
| 451 | Direction of retraction of feature 435 |
| 452 | Exemplary embodiment of outer arm cut from a tube |
| 455 | Advancement of feature 435 |
| 456 | Exemplary embodiment of outer arm cut from a tube |
| 475 | Exemplary embodiment of inner arm cut from a concentric tube |
| 479 | Exemplary embodiment of inner arm from a concentric tube |
| 482 | Outer base |
| 490 | Alternate exemplary embodiment of the valve repair device |
| 492 | Collar or sliding shaft that is detachable and attached to the delivery catheter shaft |
| 494 | Mandrel |
| 496 | Delivery Catheter Shaft |
| 498 | Gripper (cut from part of pusher feature 501 or attached to pusher feature 501) |
| 501 | Pusher (can be made from the same sheet metal as 498 or can be different part) |
| 503 | Suture |
| 505 | Outer Arm, superelastic or elastic providing robust leaflet coapting and cinching bias |
| 507 | Outer Arm, superelastic or elastic providing robust leaflet coapting and cinching bias |
| 509 | Base mandrel |
| 511 | Slight bend at the tip of the Outer Arm 505, 507 |
| 513 | Grasping movement due to self-bias exhibited by Gripper 498 |
| 515 | Release Bar |
| 520 | Catheter delivery interface (release bar assembly) |
| 522 | Barb point design of the MitraClip ® |
| 523 | Rounded and blunt shaped barb |
| 524 | Flattened and blunt shaped barb |
| 525 | The angle between arms at the tip |
| 526 | W-shaped blunt shaped barb |
| 527 | Angle of the arm at the base |
| 529 | Distance between arms at the tip |
| 531 | Distance between arms at the base |
| 532 | Detachable Tether attached to the inflatable member sealable aperture |
| 535 | Self-sealing attachment interface to the tether 532 |
| 540 | Mitral Valve with MR and implant |
| 542 | Regurgitant Gap in Mitral Valve 540 |
| 544 | Expandable or Inflatable Member or spacer attached to Gripper 309, to reduce regurgitant gap 542 |
| 546 | Single leaflet engaging implant Arm 307 and Gripper 309 assembly with feature 544 |
| 548 | Expandable or Inflatable Member |
| 550 | Center posts on the Release Bar |
| 552 | Expandable or Inflatable Member |
| 555 | Device along with the implant and catheter inserted through the LA into the LV |
| 557 | Steerable Guide Catheter |
| 559 | Steerable Sleeve Catheter |
| 561 | Delivery Catheter |
| 563 | Distal sheath covering the valve |
| 564 | Expandable valve |
| 565 | Left Lateral Arm |
| 567 | Right Lateral Arm |
| 569 | Ventricular Suture to actuate the Arms 565, 567 |
| 573 | Atrial Suture for release or expunge the leaflet from the device |
| 577 | Stopper |
| 579 | Bump with a ramp |
| 581 | Pull Wires/Circular wires for 4-way steering in the distal curve |
| 583 | Stiffener to prevent curving in horizontal plane |
| 585 | Stiffener feature/rectangular strip to prevent curving in orthogonal plane and to acuate the proximal curve. |
| 587 | Proximal Shaft |
| 589 | Stainless steel Steerable Guide Handle mount |
| 590 | Steerable Guide Catheter |
| 591 | Fully adjustable/lockable one hand hemostasis valve |
| 592 | Delivery Catheter |
| 593 | Flush Port |
| 594 | Proximal 2-way steering |
| 595 | Distal 2-way steering (A/P curve) |
| 596 | Distal 2-way steering (M/L curve) |
| 597 | Arm Actuation Rod |
| 598 | Gripper Actuation Rod |
| 599 | Bailout Actuation Rod |
| 600 | Release Knob |
| 601 | Flush port of DC Handle |
| 603 | Steerable Guide Catheter |
| 605 | Funnel-shaped expandable/collapsible implant 609 capture feature |
| 606 | Fan-shaped Mesh |
| 607 | Site where 605 is attached to the delivery catheter |
| 609 | Implant |
| 611 | Delivery Catheter |
| 613 | Maximum distance between the point of deployment and the implant |
| 615 | Maximum distance between fully expanded funnel-shaped mesh and the implant |
| 621 | Coiled leaf spring |
| 623 | Maximum distance between the coiled leaf spring and the implant |
| 625 | Coiled spring over the Delivery Catheter 611 |
| 630 | Delivery system with a feature 605, 621 for retracting the implant |
| 640 | Site where coiled spring 625 is attached to the delivery catheter |
| 635 | Distal end of the rescue catheter shaft with a slit |
| 650 | Rescue catheter proximal shaft |
| 670 | Slit |
| 690 | Static or dynamic, actuatable or remotely controllable expandable feature |
| 700 | Rescue catheter |

### DETAILED DESCRIPTION OF THE INVENTION

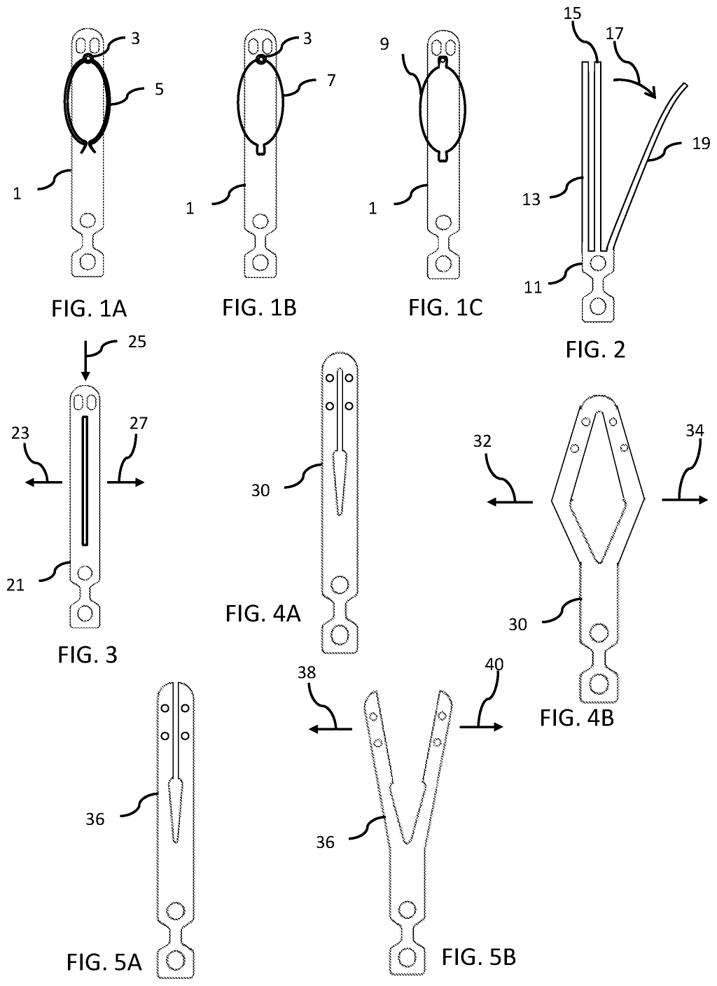

FIG. 1A-1C show exemplary embodiments of an outer arm 1 comprising of a Nitinol wire fashioned into ring 5 that is located behind the outer arm. Ring 5 is attached to outer arm 1 via screw 3; however, ring 5 may also be attached by a suture or welded to outer arm 1. The ring can be compressed while in delivery catheter. When outside of the delivery catheter, the expanded ring provides additional surface area for grasping and/or mechanical strength. The ring can be a single circular wire, such as rings 7, 9, wherein a segment is fixed on the proximal end of the outer arm and a free-standing segment on the distal end of the outer arm. Rings 7, 9 can also be made of multiple coils, patterns, and shapes.

FIG. 2 illustrates an alternative configuration of outer arm 11 with tines 13, 15, 19. Tines 13, 15, 19 can be actuated and/or be configured to elastically self-expand (using superelastic materials such as nitinol) in multiple combinations such as in pairs or singles, or in different directions and orientations, to provide additional support and strength.

FIG. 3 shows an alternative configuration of outer arm 21 with stent patterns and properties. Like a nitinol stent, outer arm 21 can be configured to self-expand as shown by arrow 23, 27 as it exits the guide catheter, thus fore shortening (arrow 25) in length. The stent-like outer arm can have an open and/or closed cell design and can be applied to the inner arms as well.

Similarly, FIG. 4A illustrates outer arm 30 with an elongated slot that expands laterally. FIG. 4B shows an exemplary direction (arrows 32, 34) in which the outer arm has self-expand. Lateral expansion (of outer arm 30) increases surface area in which to grasp the leaflets and provides additional mechanical support.

FIG. 5A shows split outer arm 36 that can be configured to self-expand laterally to increase the amount of tissue grasped. FIG. 5B shows the direction (arrows 38, 40) in which the outer arm self-expands.

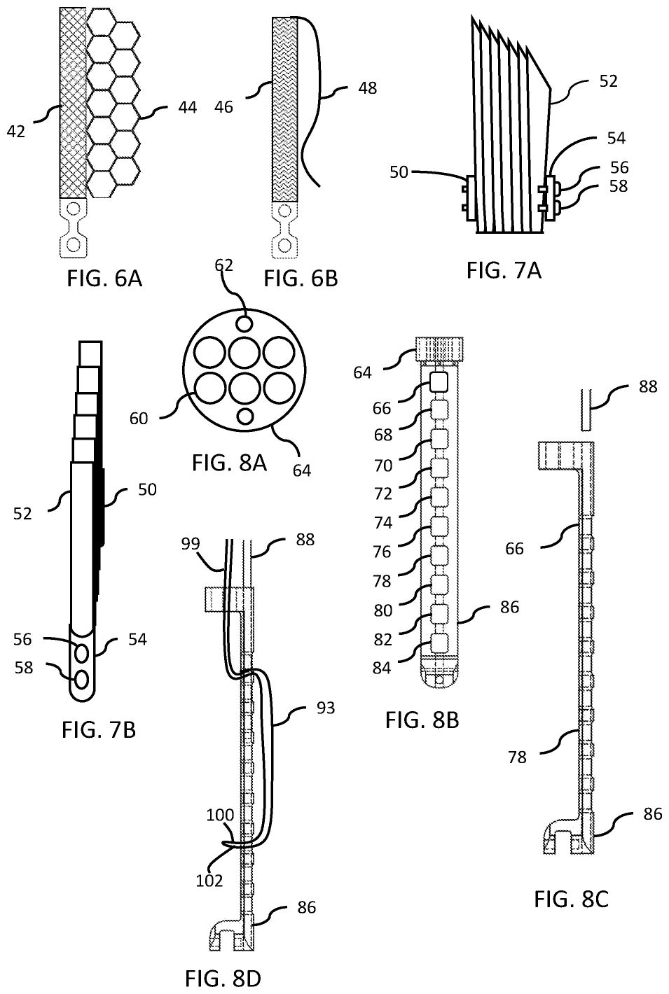

FIG. 6A shows an exemplary embodiment of an outer arm 46 comprising of a stent pattern, for example honeycomb pattern 44. The stent pattern may be planar, 3D, rectilinear, grid, triangular, S, Z, V, W, partial honeycomb and/or full honeycomb. The addition of stent pattern 44 allows additional area to grasp the leaflets. Further, these patterns allow for laterally and/or radially expanding arms that can be manufactured from strip metal, sheet metal, tubes, and/or wire materials. The stent patterns may also be applied to the inner arms. FIG. 6B shows additional features/coating that can be added to outer arm 42 to increase its mechanical, chemical, biological, and/or electrical properties, such as wires, drug-eluting polymers, sensors and/or actuators, stent patterns like feature 44, and so forth. For example, a spring wire 48 is attached to outer arm 46 to increase mechanical properties and surface area. As such, outer arms 42, 46 may be composed of various combinations of stent patterns, components, and plurality of outer and/or inner arm layers.

For example, FIG. 7A shows an alternative embodiment of the device comprising of outer bases 52, 50 and inner arm 54 in a folding fan-like (hinged Japanese fan) configuration in which outer arms 42, 46 may also be used. An alternate exemplary stacked leaf-spring configuration can be akin to truck leaf-springs. FIG. 7B shows a front view of the said device in FIG. 7A.

FIG. 8A shows the top view of the proximal portion 64 of release bar 86 comprising of a plurality of opening 60 for the insertion of actuation suture/wire and a plurality of opening 62 for the insertion of guidewire/release mandrel. As such, bailout suture 118 (not shown) and/or secondary suture 93 (not shown) utilize the two middle openings of feature 60 for ease of access and reduction of tension and strain placed on sutures actuating the inner and outer arms. FIG. 8B shows a plurality of openings 66-84 on release bar 86, wherein suture loops can be anchored to any of the openings. FIG. 8C illustrates the schematic of the release bar 86, noting the openings 80, 68 in which secondary suture 93 (not shown) is looped through. Referring to FIG. 8D, the schematic shows secondary suture 93 looped through the openings shown in FIG. 8C and the placement of exemplary device 5. As such, secondary suture 93 does not interfere with the implant device during leaflet capture. Secondary suture 93 can be inserted through any of the openings on the release bar. FIG. 8D demonstrates secondary suture 93 through the third opening 78.

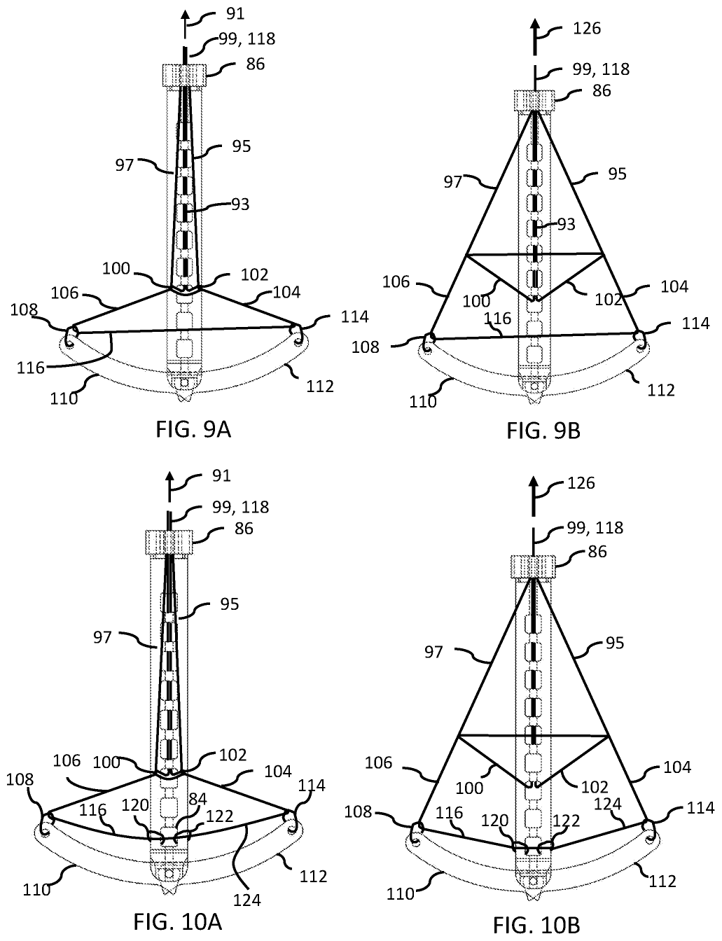

FIG. 9A shows a schematic for an exemplary bailout feature as part of the delivery system. Bailout suture 118 (not shown) is threaded through suture loops 108, 114 on inverters 110, 112, respectively. A secondary suture 93 is threaded through release bar 86 and looped around bailout suture 118. When secondary suture 93 is retracted, as demonstrated by arrow 91, bailout suture segments (106, 97, 104, 95) of suture 118 is pulled/cinched towards release bar 86, thus moving suture segments 97, 95 of bailout suture 118 away from implant device and preventing interference from the bailout suture during leaflet capture. A very small tension may be exerted on suture 118 to prevent accidental capturing of tissue and/or chordae. However, in FIG. 9B, when bailout suture 118 is retracted as demonstrated by arrow 126, while simultaneously allowing the secondary bailout suture 93 to relax, suture segments 97, 106, 116, 104, 95 of bailout suture 118 are pulled taut to expunge any tissue and/or chordae trapped between the arms/grippers of the implant device or between the implant device and inverters 110, 112 during leaflet capture. Obviously, this action should be paired with raising the inner arms (grippers) and lowering (opening) or inverting the outer arms.

In a different configuration shown in FIG. 10A, suture segments 116, 124 may be attached to release bar 86 via suture loops 120, 122 in order to provide additional clearance distance between implant device and bailout suture. As such, FIG. 10B illustrates that retraction of bailout suture 118 causes suture loops 120, 122 to maintain suture segment 116, 124 of bailout suture 118 at a lower position, further preventing interference with implant device.

Bailout suture 118 and secondary suture 93 may be coupled with spring systems so that actuating bailout suture 118 via a pull rod will stretch the spring to relax the secondary suture 93 without the need of a secondary pull rod.

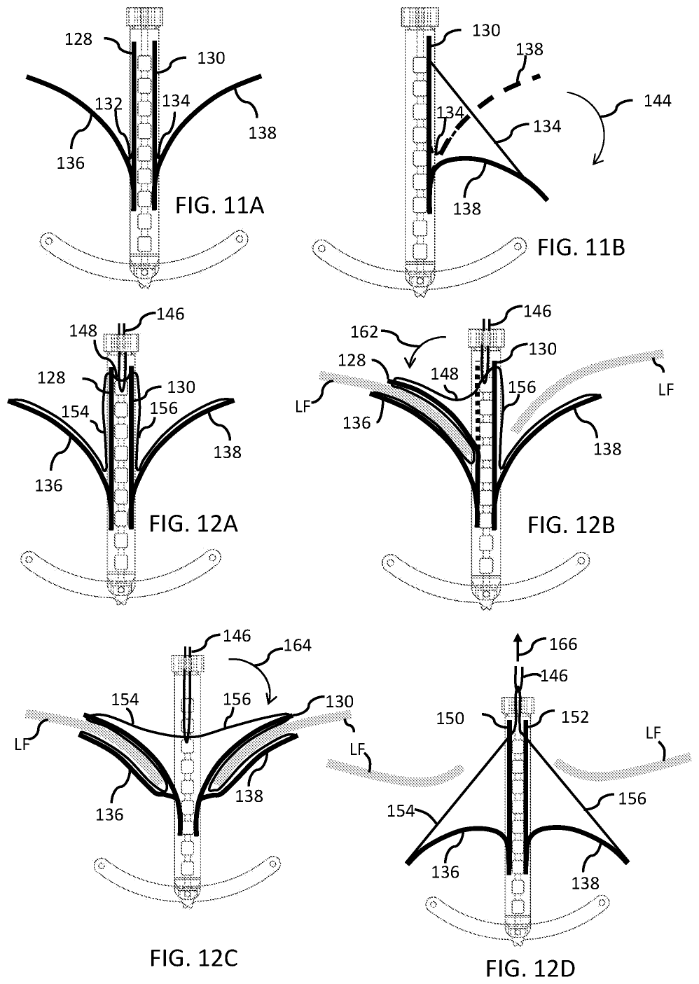

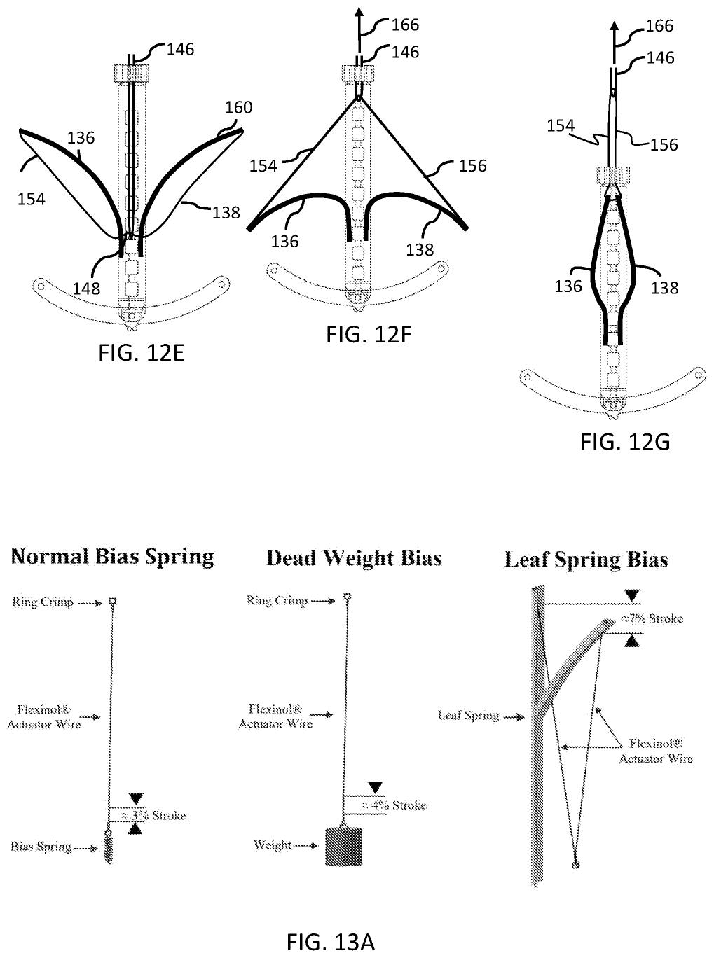

FIG. 11A illustrates a schematic for an automatic bailout feature as part of the implant device. Bailout suture 132 is looped through inner arm 128 and outer arm 136, whereas bailout suture 134 is looped through inner arm 130 and outer arm 138 of the implant device. When inner arms 128, 130 and outer arms 136, 138 are in grasping position, bailout sutures 132, 134 are slack; therefore, the leaflets can be captured without interference from the bailout sutures. Referring to FIG. 11B, when outer arm 138 is lowered to an inverted position 140, as indicated by arrow 144, bailout suture 134 is automatically pulled taut to position 142. As such, any tissue and/or chordae trapped between inner arm 130 and outer arm 138 will be expunged. Additionally, if inversion and/or repositioning is needed, the leaflets may be easily released due to the bailout suture. FIGS. 12A to 12D show alternate exemplary steps to capture and or bailout.

FIG. 12E illustrates an exemplary embodiment of an automatic bailout suture as part of the implant. Bailout suture segments 148, 154, 156 are a continuous suture threaded and/or passing through the fabric of the inner and outer arms and is configured such that it remains slack unless actuation suture 146 is retracted. Actuation suture 146 is designed to be a part of the delivery system; it is configured to loop around the apex of bailout suture segment 148. When the outer arms of the implant device are inverted and actuation suture 146 is retracted, bailout suture segments 148, 154, 156 are pulled taut in a triangular formation so that caught chordae and/or tissue are expelled from between the inner and outer arms (FIG. 12F). FIG. 12G shows an exemplary configuration, wherein, the bailout suture 146 is retracted considerably, until it draws the outer arms together. Note: Inner arms/Grippers are not shown in all of the FIGS. 12E to 12G for simplicity.

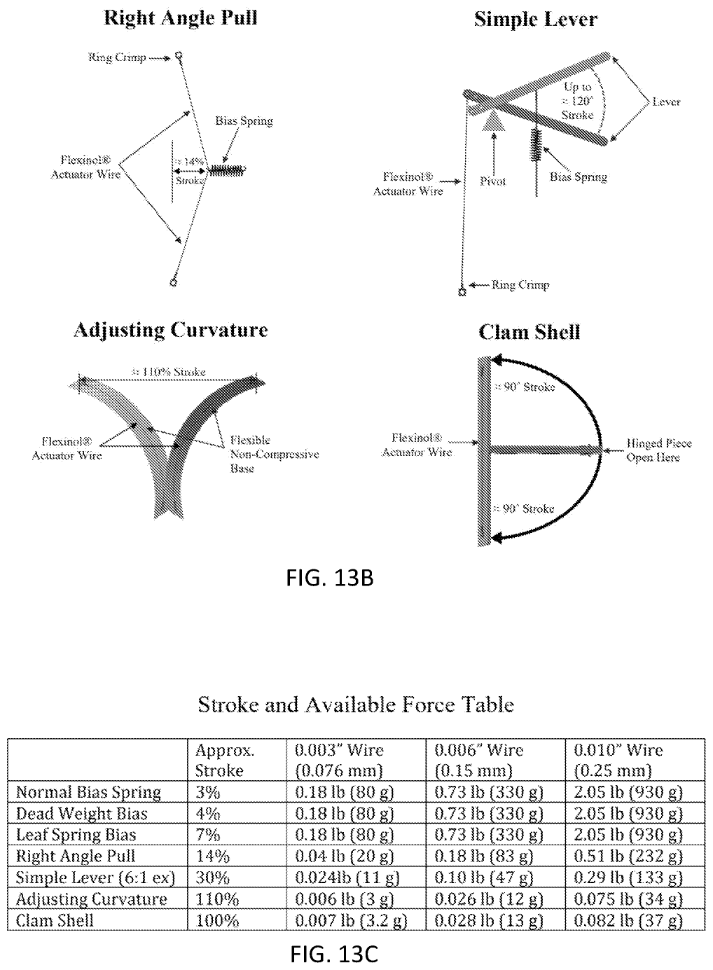

FIGS. 13A to 13C show common methods of using nitinol actuators or motors.

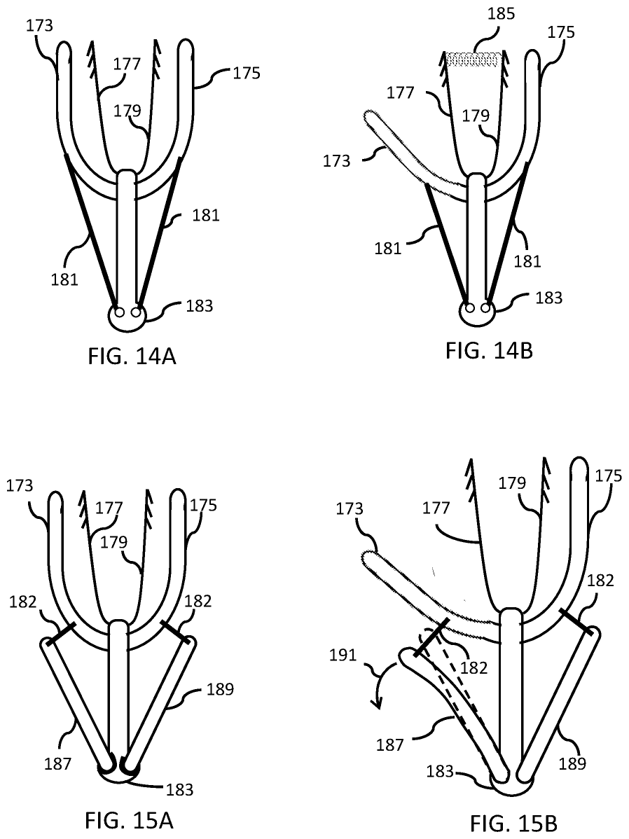

FIG. 14A shows an exemplary embodiment of a tissue fixation device wherein the nitinol outer arms 173, 175 are actuated with nitinol motor/actuator strip and/or wire 181. When an electric current or heat is applied to wire 181, it shortens and the self-closing nitinol outer arms 173, 175 are pulled to grasping position as shown in FIG. 14B. Likewise, wire 181 lengthens when it is allowed to cool down, causing outer arms 173, 175 to reposition back to its original closed shape.

In another configuration of an electrically actuated device, FIG. 15A demonstrates a device with a pair of Nitinol leaf springs 187, 189 positioned at base 183 and are tethered to nitinol outer arms 173, 175 via wire and/or suture 181. Leaf springs 187, 189 can be manufactured from any superelastic or shape memory material, such as Nitinol. Applying an electric current or heat through leaf springs 187, 189 will cause them to bend, thus pulling the outer arms into grasping position, as shown in FIG. 15B. The degree of stroke will be dependent on the heat imparted in order to precisely position the leaf springs in various configurations (i.e. grasping position and bailout).

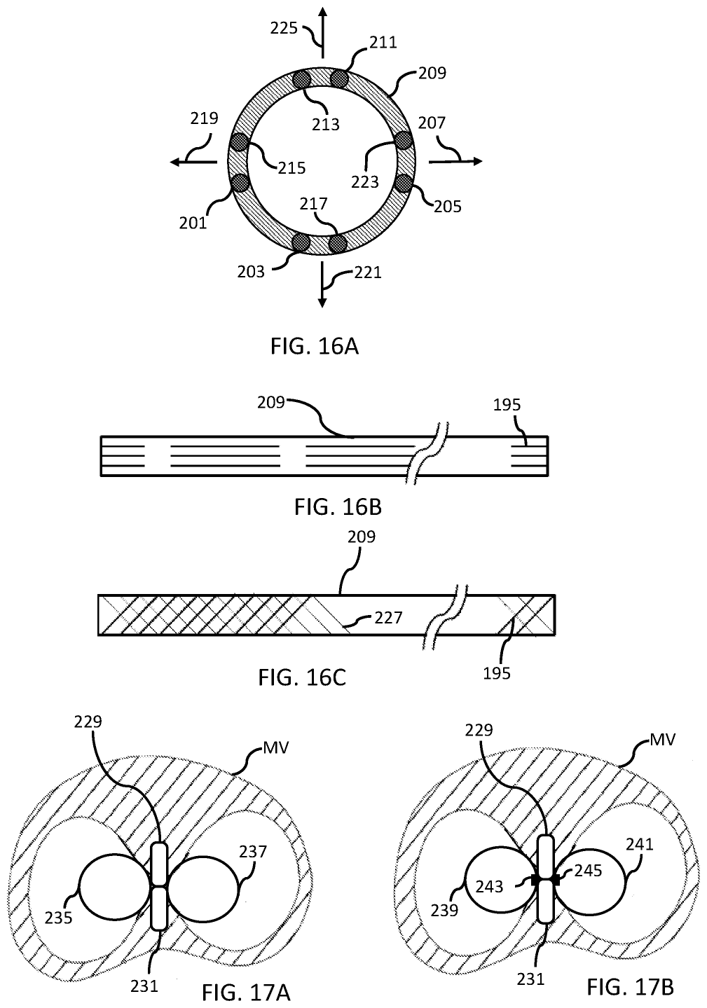

FIG. 16A shows a diagram of an electrically operated actuator wire 195 embedded in a flexible non-compressive material 197 in an adjusting curvature structure. Actuator wire 195 can have a stroke between 0-110%. The actuator wires function by contracting with heat and relaxing when cooled. FIG. 16B illustrates a frontal view of a plurality of actuator wires 213, 211, 223, 205, 217, 203, 201, 215 embedded within an electrically driven catheter 209. Depending on the function of catheter 209, actuator wire 195 can vary in size, geometry and plurality. Additionally, actuator wire 195 can be embedded and/or positioned anywhere in the inner and/or outer diameter of the catheter and/or catheter lumen(s). Electrically heating specific actuator wires will cause the wires to move to a specific position. For instance, electrically stimulating actuator wires 223 and 205 or actuator wires 201 and 215 will move catheter 209 in the lateral direction (arrows 207 and 219, respectively). Likewise, electrically heating actuator wires 213 and 211 or actuator wires 217 and 203 will move the catheter in the vertical or downward direction (arrows 225 and 221, respectively). Thus, heating certain combinations of actuator wires will move the catheter in the wires' respective planes. As such, a simple circuit is used to contract the catheter and maintain the desired position. The actuator wires are used in conjunction with a non-conductive and temperature resistant material, such as silicone rubber, ptfe, fpa, polyimide, and or peek, in order to overcome the wire's high temperature and high force during operation.

FIG. 16C shows a longitudinal view of catheter 209 with actuator wires 195 in a parallel configuration. As discussed in FIG. 16B, actuation of specific wires will cause motion in the catheter along the wires' respective planes. FIG. 16C shows the actuator wires 195 and actuator wires 227 in a crisscross configuration, causing catheter 209 to twist and bend with the actuation wires that are activated. For instance, an actuator wire will contract at an Austenite finish (Af) temperature between −45° C. to −50° C. and lengthens at 37° C. to 40° C. Catheter 209 can be comprised of solely parallel actuator wires, crisscross wires, or a combination of both configurations in any permutation and design. Additionally, the wires can be any size, geometry and plurality. By using microprocessor controller, the nitinol motor-based catheter can be operated similar to fluidic catheter described in U.S. Pat. No. 10,500,373 (and related family of patents, including all patent applications from the assignee of the patent), incorporated and referenced here in its entirety

Actuator wires can be utilized to move the inverters of the delivery system or the implant. Using a leaf spring bias mechanism can actuate the inner and outer arms with a stroke of −7% or more depending on the bias force. As for the inverters, a simple lever or clam shell mechanism can result in a stroke of −120% or −90%, respectively. Stroke for these mechanisms can be improved with designs that have reverse bias force.

FIG. 17A illustrates a mitral valve with a clip fixed on the leaflets to form a double orifice. The clip has one or more, for example two balloon features 235, 237 that are detachable and inflatable. Balloons 235, 237 are inflated via a tube connected and controlled by a delivery catheter. Once balloons 235, 237 are inflated to their recommended volume, the inflation tube will be detached from the delivery catheter and gradually detaches from balloons 235, 237 themselves. Moreover, the delivery catheter can manually inflate or deflate the balloons individually and/or simultaneously. In the case of FIG. 16A, balloons 235, 237 are static, meaning they maintain their volume after the inflation tube detaches. However, FIG. 17B illustrates a similar clip with dynamic balloons 239, 241 wherein the balloons can be controlled via mechanical and/or electrical stimuli (i.e. heartbeat) or remote control (i.e. smartphone). Ideally, this system will be used for high-risk patients with mitral valve deformities wherein the mitral valves don't fully close. In cases of these patients, current practice involves the use of multiple clips which may not be recommended. As such, balloons 239, 241 will use actuators and/or sensors 243, 245 and will inflate during systole and deflate during diastole, ie. synchronous with heartbeat. Alternatively, the extent of inflation and deflation can be variable and tailored to the real-time needs of the patient for example, during sleeping, walking, resting, exertion. The data will be recorded on the clip via microelectronics, actuators and/or sensors 243, 245 and relayed to an external source (i.e., smart electronics). Unlike the clip of FIG. 17A, this clip can have an embedded inflation mechanism located anywhere on the clip's exterior. Inflation and/or deflation of these balloons can be caused by mitral valve movement, microprocessors, microcontrollers and so forth. Balloons 235, 237 and balloons 239, 241 can be positioned on the proximal portion of the clip's base or anywhere along the clip. All embodiments of this clip and its features are claimed and expanded from referenced applications PCT/US2018/041016 and PCT/US2017/042003. Additionally, the balloon features may comprise of features listed in patents such as U.S. Pat. No. 7,854,745B2, U.S. Pat. No. 9,173,758B2 and/or U.S. Pat. No. 9,351,862B2.

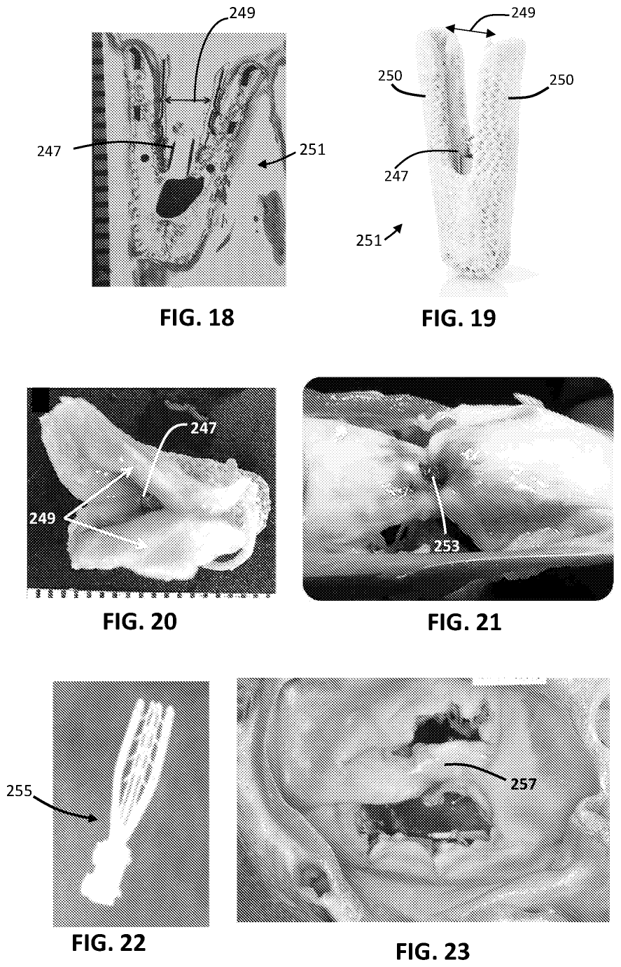

FIG. 18 illustrates a histopathological cross-section of the MitraClip® device 251, showing a large inherent gap 249 between arms and exposed bare metal components 247. FIG. 19 and FIG. 20 show a picture of fully closed MitraClip® device, showing large inherent gap 249 and exposed bare metal components 247. Blood can clot 253 in the pocket over the metal components 247, as shown in FIG. 21. On the other hand, implant 255 has very small (<1 mm) or almost no gap between arms, snugly coapting the leaflets at the tip, enabling a well endothelialized and smooth tissue bridge formation 257, as shown in FIGS. 22 and 23.

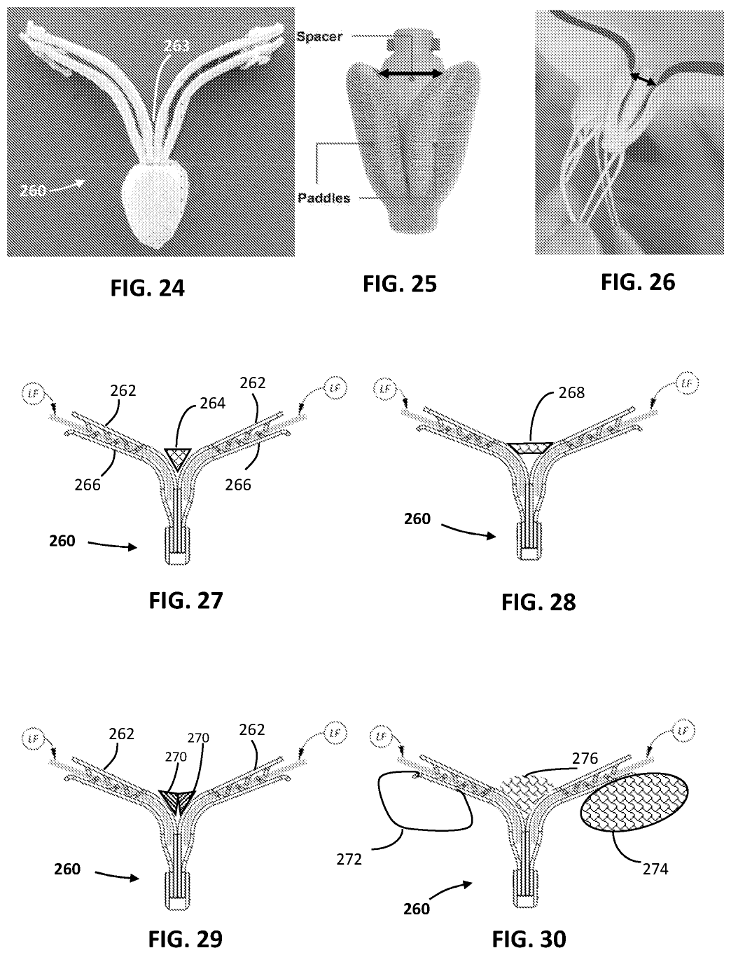

FIG. 24 shows an exemplary curved implant 260 with a pocket 263 between the curved grippers 262.

FIGS. 25 and 26 slow the Pascal implant, which is essentially a spacer concept rather than edge-to-edge technique. Unlike MitraClip®, the spacer fills the large gap in between the leaflets as seen in FIGS. 25 and 26, to mitigate thromboembolism risks. However, like MitraClip®, there is by design suboptimal cinching and coaptation of the leaflets. Hence, long-term benefits related to positive reverse remodeling of the heart remains to be evaluated. This is because effective cinching of leaflets is known to be the primary contributor for long-term positive reverse remodeling of the heart.

FIGS. 27, 28, and 29 illustrate various exemplary embodiments of innovation to reduce the pocket between the grippers using spacers 264, 268, 270, 272, 274, 276. The grippers together form a cleft or gap where they join over the hub, and the cleft is susceptible to clotting and thrombus formation. A spacer 264, 268, 270, 272, 274, 276 can be an expandable and or compressible sponge, mesh, balloon, non-thrombogenic fabric, etc., as evident to those skilled in the art. These spacers can be attached to the atrial side of the grippers or to ventricular side of the arms to obtain the bridge structure between arms. Having a spacer between arms reduces the risk of thrombus formation by decreasing the recirculation zone where blood elements can get trapped in this zone and experience high shear stresses for a long period result in thrombosis and thromboembolism.

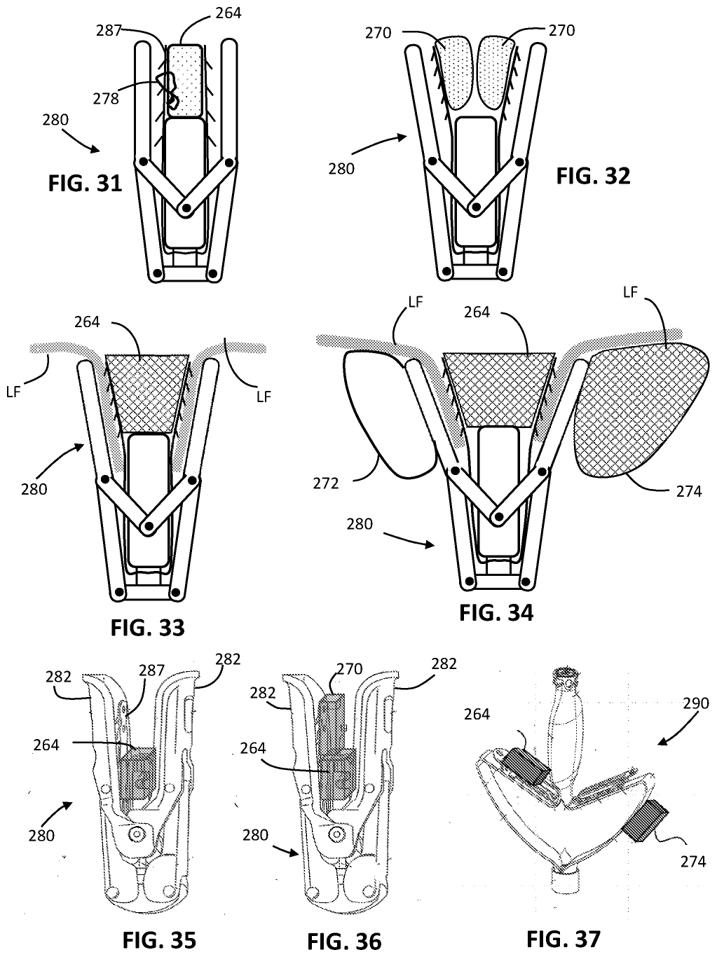

FIGS. 31-36 depict exemplary embodiments of innovation to reduce the inherent gap between the tip of the arms 282. In FIGS. 31, 33, and 35, a large exemplary spacer 264 is fastened to the atrial side of central post or gripper using suture, bond, weld, glue, and/or fastener. FIGS. 32 and 36 show the inherent pocket filled with two small spacers 270 where each spacer is fastened to individual gripper to decrease the gap 249. FIGS. 34 and 37 shows exemplary embodiment with atrial 264 and ventricle side leaflet support spacers 272, 274. FIG. 37 shows exemplary embodiment with spacer 264 that can replace the center spacer. Additionally, the embodiment shows ventricular spacer.

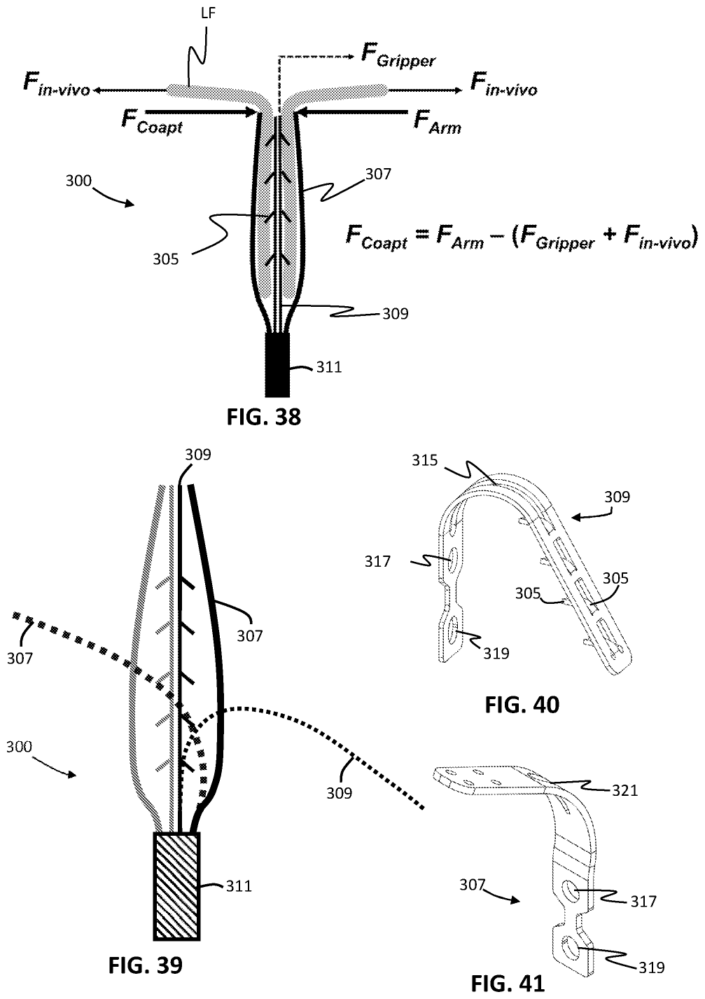

One of the problems with competitive devices is that leaflets do not adequately coapt. Furthermore, some of them are configured to have a spacer or space in between the leaflets that further keep the leaflets apart. This is contrary to the principal of the surgical Alfieri technique. In addition, cinching of leaflets help in positive remodeling of the heart. FIG. 38 shows an exemplary embodiment of device, described in co-owned patent application US20200383782A1. That is the coapting force F_{Coapt} exerted by the OuterArms 307 must significantly overcome the in-vivo forces Fᵢₙ₋ᵥᵢᵥₒ exerted by the leaflets and the annulus, as well as the Grippers F_{Gripper}. This is achieved by making the OuterArms 307 much stronger than the Grippers 309 in bending, by either increasing the thickness and/or cross-section of the OuterArms 307 compared to the Grippers 309. Furthermore, a high bias is configured by over-curving both the OuterArms 307 and Grippers 309, as shown in FIGS. 39, 40, and 41. In some embodiments, the thickness of the OuterArms is preferably about 0.33 mm. In some embodiments, the thickness of the OuterArms is between 0.03, 0.06, 0.12, 0.16, 0.20, . . . , 3.12, and/or 10 mm. In some embodiments, the thickness of the Grippers is preferably about 0.20 mm. In some embodiments, the thickness of the Grippers is 0.003, 0.06, 0.12, 0.16, 0.20, . . . , 3.12, . . . and/or 10 mm. In some embodiments, the cross-section at bend region of the OuterArms is greater than Gripper bend cross-section by 0, 1, 2, 3, . . . , 999, and/or 1000%. In some embodiments, the F_{Coapt} is preferably about 0.151bf. In some embodiments, the F_{Coapt} is between 0, 0.1, 0.2, 0.3, . . . , 19.9, . . . , and/or 601bf. In some embodiments, the distance between the leaflets at the tip of the device is about or in between 0, 0.1, 0.2, 0.3, . . . , 4.9, . . . , 9.9 and/or 10.0 mm, preferably less than 1 mm. In some embodiments, when assembled the Arms and/or Grippers may be configured to have a self-biasing strain of about 0, 0.1, 0.2, 0.3, . . . , 19.9, and/or 20%, preferably between 1% and 6%. In some exemplary embodiments, the Outer Arm is bent at the tip, as in FIG. 55, in a configuration that maximizes leaflet coaptation.

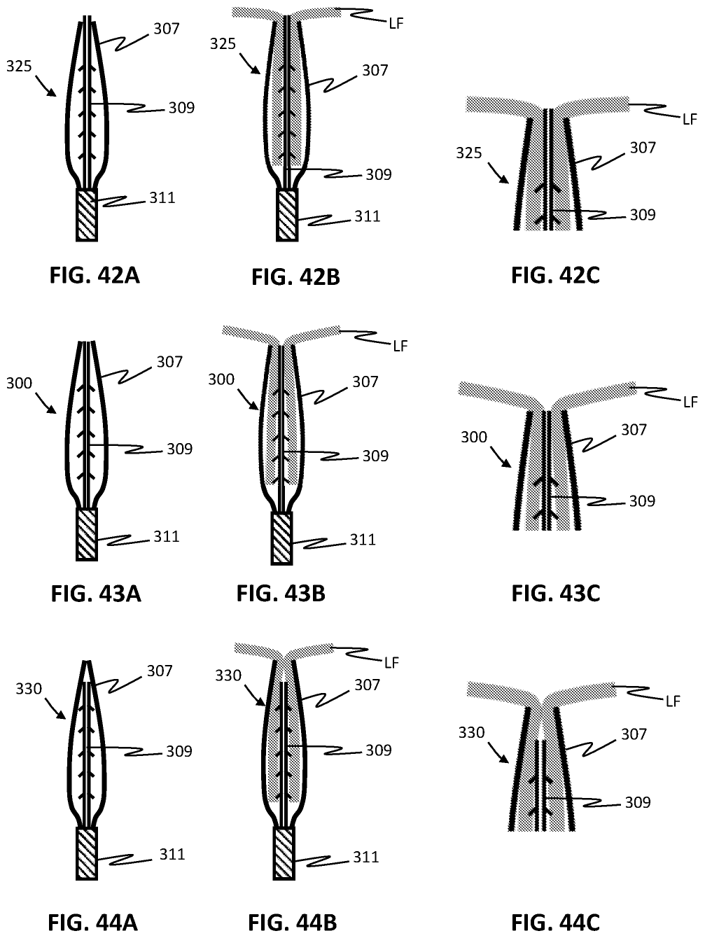

FIGS. 42A to 44C show various exemplary embodiments of tissue grasping devices, with different lengths of inner arms 309 vs. outer arms 307, that may be used attain desired coaptation configurations of the leaflets.

FIG. 42A Inner arms are slightly longer than the outer arms which facilitates a small space between the two leaflets.

FIG. 42B. depicts the space between the leaflets defined by the two inner arms. The space in between the leaflets is no greater than the thickness of the leaflets itself.

FIG. 42C shows the zoomed region between the leaflets, wherein the leaflets and inners arms are flush, with no pockets.

FIG. 43A Inner arms and outer arms are of the same height.

FIG. 43B depicts the space between the leaflets. An angle is formed by the leaflets is the result of the same height of the inner and outer arms. The space in between the leaflets is no greater than the thickness of the leaflets itself.

FIG. 43C shows the zoomed region in between the leaflets. Full cinching of the leaflets is achieved with minor pocket. Tissue encapsulation can happen in between the leaflets.

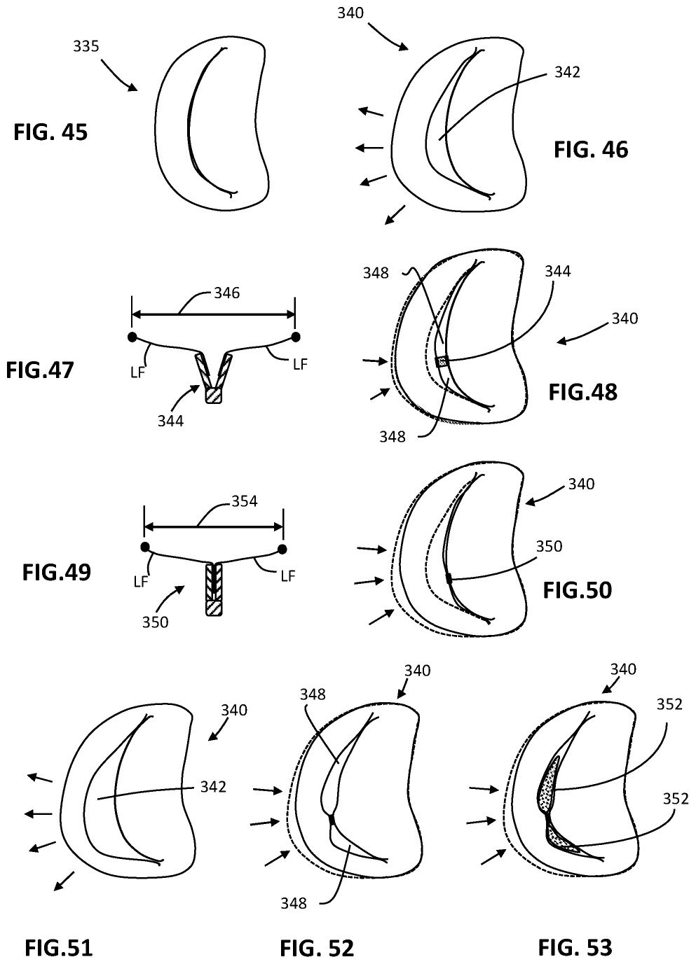

FIG. 44A Inner arms are lower than the outer arms. The outer arms are touching each other in this configuration.

FIG. 44B Both the grasped leaflets are in contact with each other as there are no inner arms separating them at the tip.

FIG. 44C Full cinching of the leaflets is achieved. Zero gap between the leaflets. The leaflets are touching each other. Tissue growth and callus formation can happen in between the leaflets.

In some embodiments, the gap between the leaflets is preferably <1 mm. In some embodiments, the gap width is 0.0, 0.01, 0.06, 0.12, 0.16, 0.20, . . . , 3.12, . . . and/or 10 mm

In some embodiments, the dip/pot-hole/grove between the leaflets is preferably <1 mm. In some embodiments, the dip/pot-hole/grove depth is 0.0, 0.003, 0.06, 0.12, 0.16, 0.20, . . . , 3.12, . . . and/or 10 mm.

FIG. 45 shows schematic of a normal mitral valve in systole, while FIG. 46 shows a mitral valve with regurgitation. FIG. 47 shows a representative schematic of a MitraClip® closed and deployed in a typical V-Shape, and thereby, having larger diametric annular distance 346 due to suboptimal cinching and coaptation of the leaflets. FIG. 48 shows that suboptimal cinching and coaptation results in suboptimal reduction of MR, due to ineffective coaptation, as depicted by the gaps 348.

On the contrary, the FIG. 49 shows implant with completely closed clip 350, with smaller diametric annular distance 354. FIG. 50 depicts superior cinching and coaptation of the leaflets result in superior apposition of the leaflets throughout the mitral valve. Thereby, resulting in improved reduction in MR or improved efficacy over the MitraClip® device.

FIG. 51 shows a schematic of mitral valve regurgitation with large gap 342 between the leaflets.

FIG. 52 shows an exemplary scenario wherein some gap 348 is present despite completely cinched and coapted leaflets. FIG. 53 shows a mitigating exemplary configuration of this invention, wherein, additional lateral or sideways protruding spacers 352 are deployed to fill the gap 348 and thereby reduce the MR. In this case, the size of the spacer is not limited to the width of the implant. The spacer 352 can be expendable, compressible, flexible, remotely adjustable post deployment to reduce the MR, using engineering solutions available to a person of ordinary skill in the art (POSA).

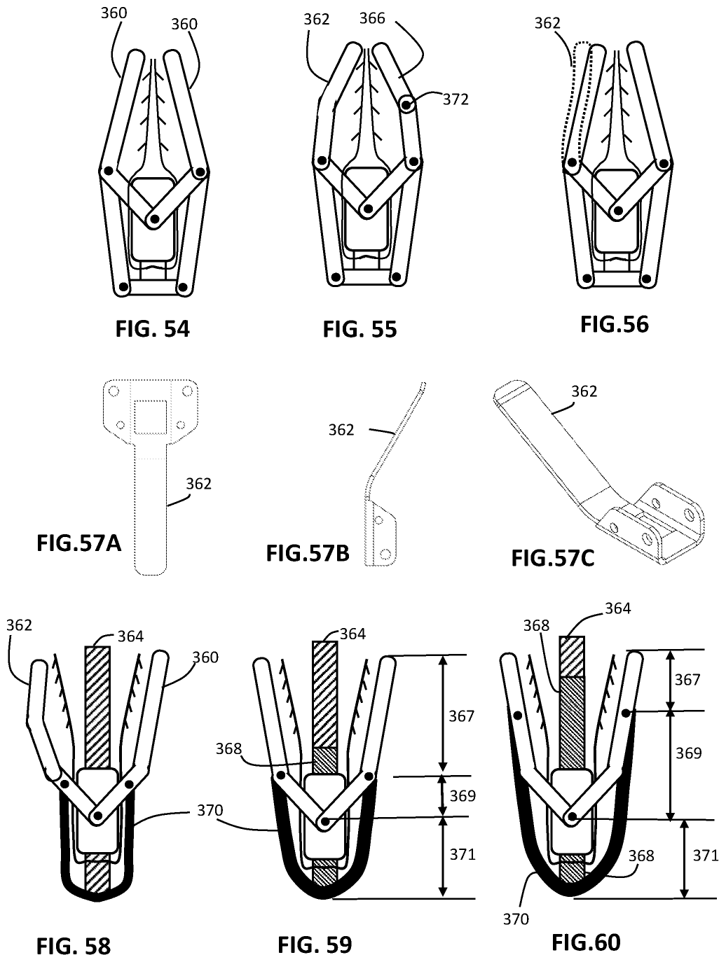

FIG. 54 illustrates the exemplary implant 280 which are angled and configured to achieve a smaller gap between the arm tips 360, when it is fully closed. FIGS. 55 and 56 show two exemplary embodiments of innovation to reduce the gap by having secondary bends in the arms in a continuous rigid system or using a spring/elastic hinge 372. Using hinge with springs or elastic arms 362 will generate a coapting spring force when closed, that acutely or progressively coapts the leaflets overtime time due to potential leaflet remodeling, post deployment. FIGS. 57A-C depict a schematic of the elastic or superelastic flexible arm 362 made of sheet metal. The entire arm can be configured to be flexible, or the distal and proximal end of the arm can have semi-rigid or U-channel and only the center can be flexible.

In FIG. 58, an inherent gap is created when the removable part of the delivery system 364 is removed. In this configuration, the U-Spring 370 applies a constant elastic coapting/cinching forces on the arms 360 to cinch and coapt the leaflets at the tip. In FIGS. 59 and 60, the inherent pocket can be fully or partially filled with an implantable portion of the actuation rod 368. FIGS. 58, 59 and 60 show the different configurations of the U-spring 370 with Arm 360, 366, 362. Each ratio variation has certain mechanical advantages, and any combination of this ratio can be used to keep the arms together. Further, any residual pocket can be filled with a compressible/expandable spacer as described previously. In addition, each of these configuration have different lever arms 371, 369, 367.

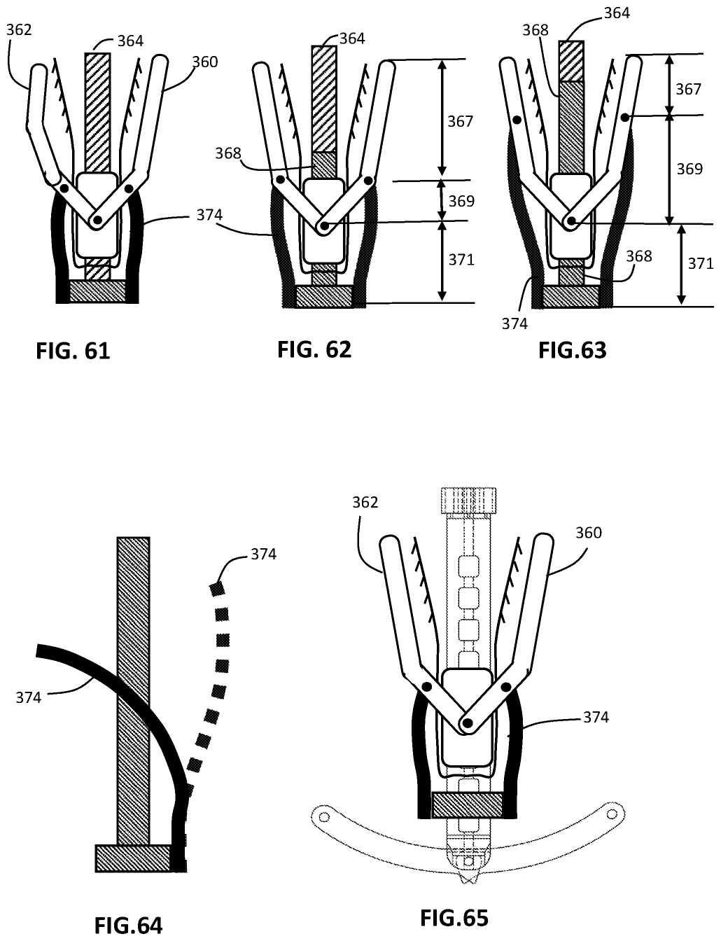

FIGS. 61, 62 and 63 show the different configurations of two leaf springs instead of a single U-spring as featured in FIGS. 58, 59 and 60. The problem with single U-spring is that by physical design, the tips of the springs cannot be formed beyond the centerline or point where both tips come in contact. As shown in FIGS. 64 and 65, individual/separate halves of springs do not have this limitation, hence, can be biased with greater strains/forces, which is a particular advantage of this invention.

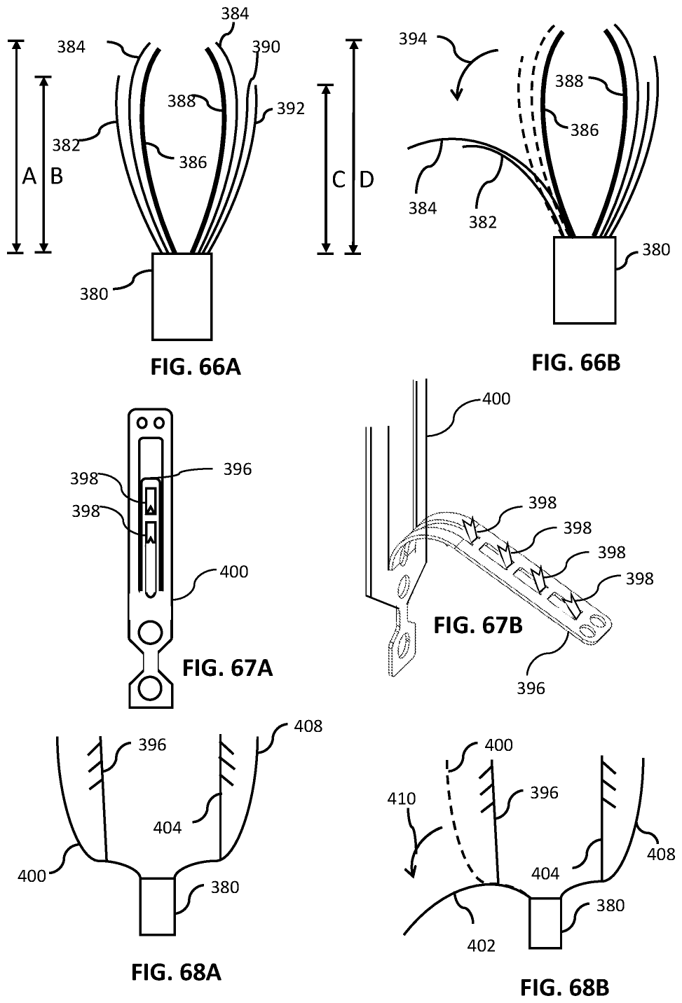

FIG. 66A shows an exemplary embodiment of a tissue fixation device with nesting leafsprings 384 382, 390, 392 of varying lengths. Nesting of the leafsprings provide additional strength/force configurations and mitigate high strain values. The leafsprings may be coated with Teflon or lubricant or current typical technology evident tot POSA to reduce friction as they are actuated/bent. Lengthwise, the leafsprings can be A=B, A<B or A>B and C=D, C<D or C>D and A=C, A<C or A>C and any other permutations. The thickness of the leafsprings can vary between outer arms 384 382, 390, 392 as well. Similarly, the inner arms can also have multiple leafsprings of varying lengths and/or thicknesses. FIG. 66B illustrates leafsprings 384, 382 actuated into grasping position as demonstrated by directional arrow 394. As can be inferred, leafspring 382 pushes against leafspring 384 to provide additional strength to function as a unit Outer arm, working together against Gripper 386, as previously discussed here and similar in function as in the referenced co-owned patents. Hence, one particular advantage of this invention is that individual leafsprings can be configured with varying lengths, thickness, and curves, to achieve desired coapting forces.

FIG. 67A illustrates an exemplary embodiment of an outer arm 400 with inner arm 396 with medial barbs, as part of said outer arm. Inner arm 396 is manufactured to be biased apart from outer arm 400 such that it grasps the mitral leaflets without the use of sutures to actuate in a closed or open position. Inner arm 396 has a plurality of barbs configured in the direction of outer arm 400. The barbs are configured to grasp the mitral leaflets within the space of outer arm 400 and inner arm 396. FIG. 67B illustrates the side view of outer arm 400 in a closed position with a plurality of medial barbs 398 on inner arm 396. Inner arm 396 is biased away from outer arm 400 to allow capture of the leaflets with atraumatic and yet robust grasping force. Barb 398 has a degree between 15 degrees and 150 degrees (preferably 60 degree) from inner arm 396 surface that allows grasping of the tissue but allows release of leaflets during regrasping attempts or bailout.

FIG. 68A illustrates a schematic of an exemplary embodiment of a tissue fixation device wherein inner arm 396 and outer arm 400 are configured as one piece and elastically biased towards each other. Outer arms 400, 408 are coupled to an outer base 380. FIG. 68B illustrates the device with outer arm 400 actuated 410 in an open position 402. Note the position of inner arm 396; it remains stationary so that when outer arm 400 is actuated to a closed position, it presses against inner arm 396 to grasp the leaflets. Alternatively, the inner arms or Grippers and outer arms or Arms can function similar to embodiments of referenced and co-owned patents.

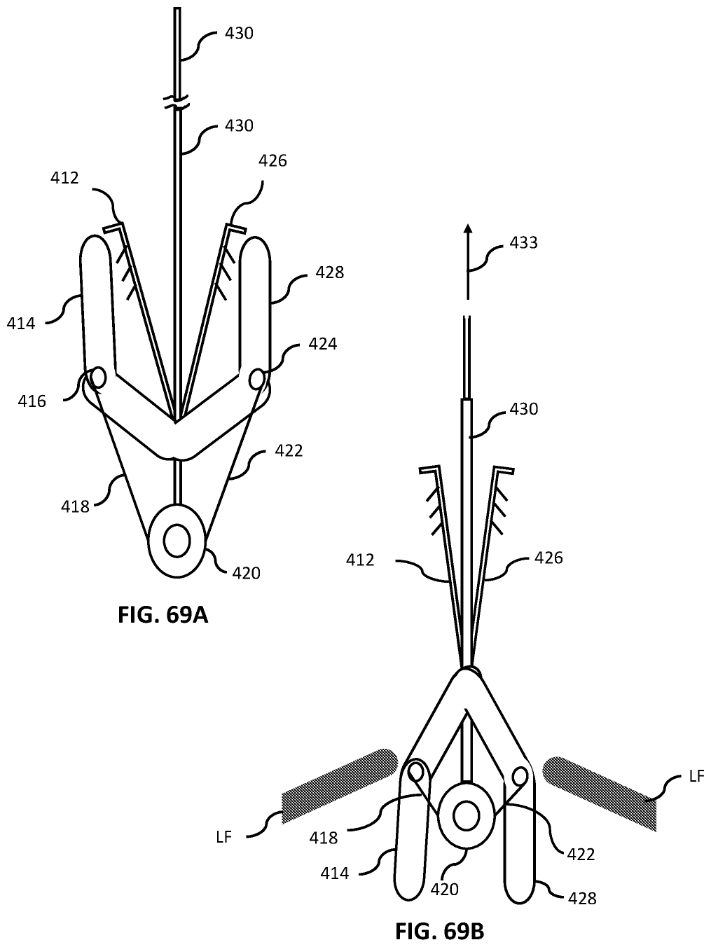

FIG. 69A illustrates an exemplary embodiment of a tissue fixation device wherein base 420 is spring-loaded to provide the compressive force needed to grasp the leaflets. Outer arms 414, 428 are actuated via sutures 418, 422 extending from eyelets 416, 424 configured at the arcs of the outer arms to the base 420. Sutures 418, 422 can be one continuous or multiple sutures or metal wires. The Outerarms 414 and 428 can be one or more components that are continuous, hinged, welded and or fastened at the base. Sutures 418, 422 are not just restrained to eyelets 416, 424; it can be attached at any position along the length of the outer arms. Pulling leaflet release rod 430 in a retracting direction will raise outer arms 414, 428, thereby releasing the leaflets from the barbs. In contrast, advancing the leaflet release rod 100 will lower the inner arms. Additionally, the base can also be a pulley mechanism and pulling or slacking the sutures 418 and 422 either simultaneously or independently/individually can raise or lower or invert the corresponding outerarm, as shown in FIG. 69B (similar to configurations described in referenced patents).

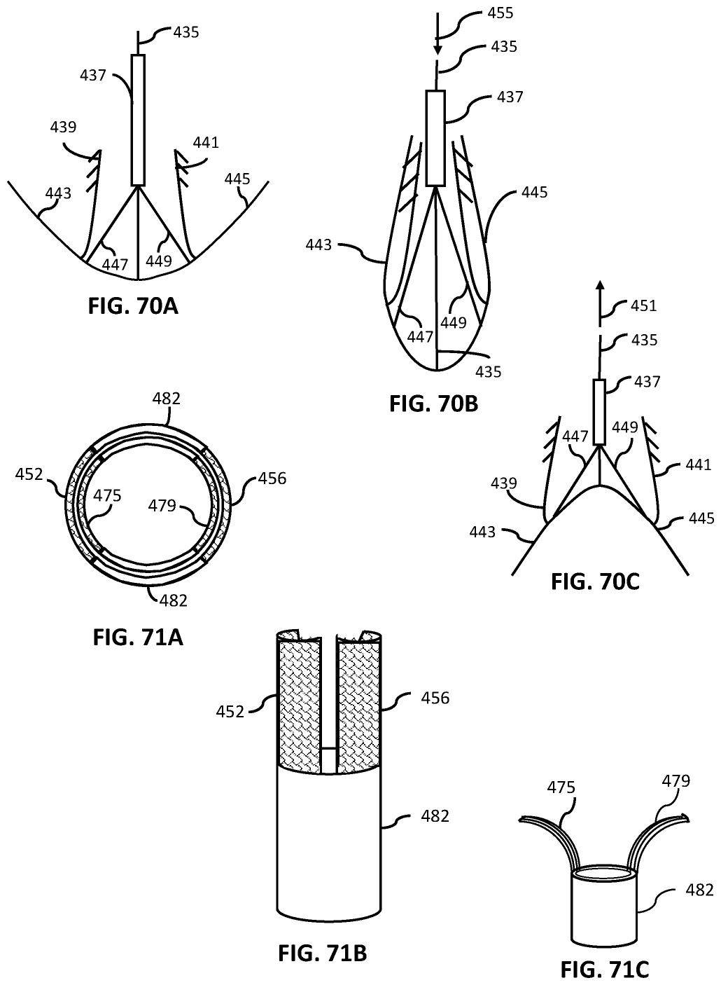

FIG. 70A shows an exemplary embodiment of a tissue grasping device wherein grippers 439, 441 and outer arms 443, 445 are one piece. Grippers 439, 441 can be screwed, welded or cut from the outer arms and actuated as in referenced patents. The outerarms are actuated via suture 435, mandrel or wire, similar to that of a common umbrella. Advancement (arrow 455) or retraction (arrow 451) of suture/mandrel/wire 435 is conducted through delivery shaft 437. Secondary struts (or beams or sutures) 447, 449 permanently or detachably connect to outer arms 443, 445 and to actuation suture/mandrel/wire 435 to allow positioning of the grippers and outer arms. Retracting the suture/mandrel/wire causes the outer arms to collapse into a closed position (FIG. 70B) and advancing the suture/mandrel/wire moves the grippers and outer arms to grasping position (FIG. 70C). Note, while not shown, it can be inferred from previously referenced and co-owned patents the mechanisms to raise or lower Grippers.

FIG. 71A shows a top-view schematic of an exemplary embodiment of a tissue fixation device configured from two or more coaxial metal tubes, which also contributes as outer base 482. Outer (452, 456) are cut from the same tube such that the device is one continuous device. FIG. 71B shows a side view of the device. The inner arms (475, 479) are formed similarly of smaller OD tube. FIG. 71C shows an alternative configuration of the same device wherein the inner and outer arms are biased outward.

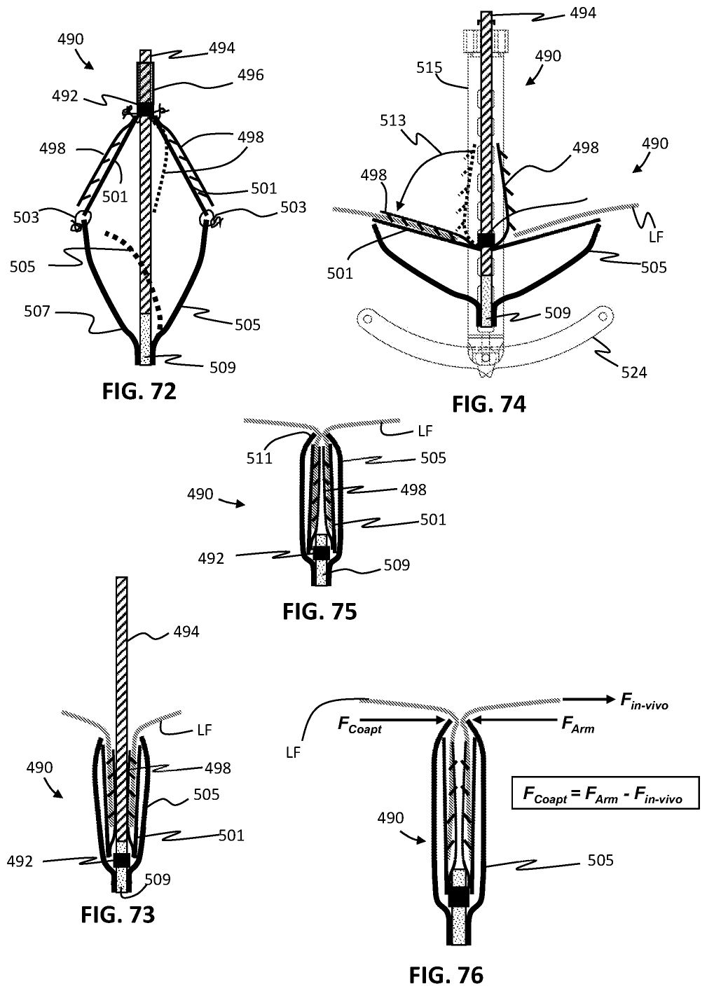

FIGS. 72-76 show an alternate exemplary embodiment of the tissue grasping valve repair device. The OuterArms 505, 507 are fastened or bonded to the BaseMandrel 509, which is detachably attached to a mandrel 494. The tip of the OuterArms 505, 507 are connected via sutures or fabric or any hinge configuration to the tip of the PusherRod 501. At the other end of the PusherRod, a Gripper may be fastened or bonded or riveted. Further, the entire sub-assembly of the PusherRod and Gripper is connected to the SlidingCollar 492 using sutures or fabric or a hinge. SlidingCollar 492 is detachably attached to the delivery shaft 496, over the Mandrel 494.

Like in the previous embodiment in FIGS. 122-125, the OuterArm 505, 507 are configured to exert significant coapting forces over the leaflet. As shown in FIG. 76, the coapting force F_{Coapt} exerted by the OuterArms 505, 507 must significantly overcome the forces by the inner arms and the in-vivo forces Fᵢₙ₋ᵥᵢᵥₒ exerted by the leaflets, which includes cinching of the anulus, as described in earlier embodiments. This is achieved by making the OuterArms 505, 507 much stronger in bending.

One problem with the PASCAL device is that it is essentially a spacer and the paddles are weak. It is not designed to coapt or cinch the leaflets. One advantage of this invention is excellent cinching of leaflets in addition to close (preferably <1 mm gap) coaptation.

By retracting the Mandrel 494 relative to Delivery Catheter 496, the OuterArms 505, 507 can be spread apart to leaflet grasping position, as shown in FIG. 72. Additional configurations are shown in FIGS. 73 to 76.

For example, as shown in FIG. 74, the device may be loaded using BaseMandrel 509 onto the RelaseBar and inverters 524 may be used spread the OuterArms 505, 507, and the Grippers 498 can be raised or lowered 513 in a similar mechanism using sutures, as described in co-owned patent application US20200383782A1, reference here in full.

The device 490 maybe loaded coaxially to the Delivery Catheter 496, mandrel 494, and BaseMandrel 509. Alternatively, the device 450 may be side mounted on the ReleaseBar 520, as in co-owned patent application US20200383782A1.

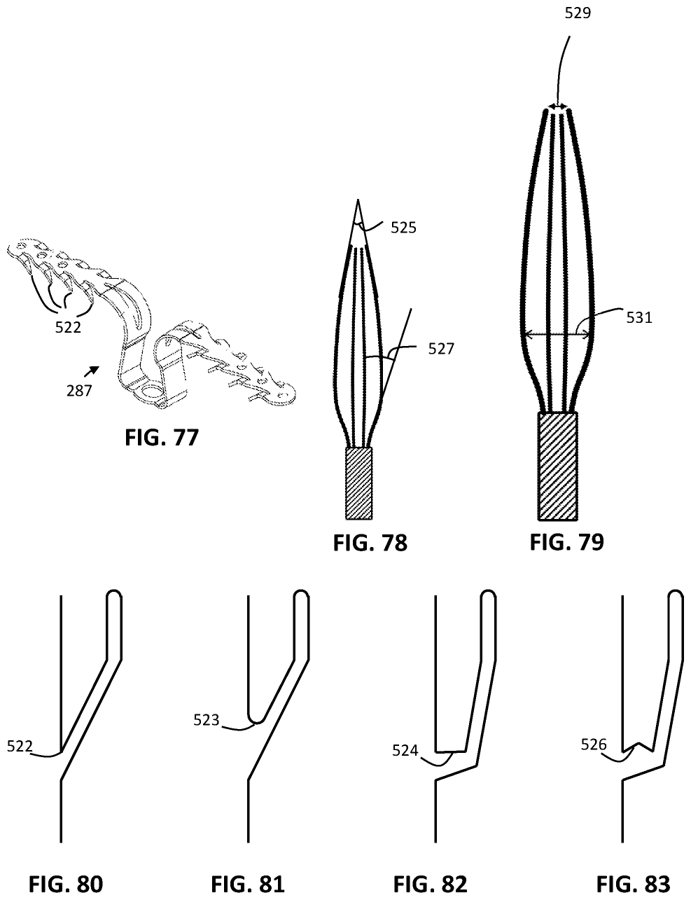

FIG. 77 shows the MitraClip® gripper 287 with sharp and exposed barbs 523 which are prone to leaflet tears, perforations and/or SLDA. Exemplary embodiments in FIGS. 80 to 83, show various versions of atraumatic designs comprising of blunted points 523, 533, 535, 537 to mitigate the leaflet trauma. These innovative barb designs may comprise of other leaflet trauma mitigations evident to POSA, such as tissue penetration limiting features to prevent leaflet tears, perforations and/or single leaflet device attachment (SLDA).

The FIG. 78 illustrates the angle between the outer arms at the base 527 and tip 525 of the device. This is needed to ensure pocket-less and smooth coaptation of the leaflets at the tip.

FIG. 79 shows the width at base 531 and top 529 of the clip. A larger base width is needed to accommodate thicker leaflet edges and smaller width is needed to ensure pocket-less and smooth coaptation of the leaflets at the tip.

The following lists various exemplary configurations of the invention. Here are the potential configuration variations in arm angles and widths, as in FIGS. 78 and 79.

Angle between the Tips of the Outer Arms=−90, −60, −45, −30, −15, −10, −5, 0, 5, 10, 15, 20, 25, 30, 45, 60, and/or 90 degrees. Preferred angle=−10 to 30 degrees

Angle at the base of the Outer Arms=−90, −60, −45, −30, −15, −10, −5, 0, 5, 10, 15, 20, 25, 30, 45, 60, and/or 90 degrees. Preferred angle=−10 to 30 degrees

Base (or Arm, and/or Gripper) width≥top width; Base width=top width; base width≤top width

Base Base (or Arm, and/or Gripper) width≥ or ≤top width by 0.0, 0.1%, 1%, 10%, 25%, 50%, 75%, 100%, 150%, 200%, 500%, 1000%, and or 10,000%

Base Base (or Arm, and/or Gripper) width and/or top width=0.0, 0.01 mm, 0.1 mm, 1 mm, 1.5 mm, 2 mm, 2.5 mm, 3 mm, 4 mm, 5 mm, 6 mm, 10 mm, 20 mm, 30 mm, 50 mm, 100 mm and/or 300 mm

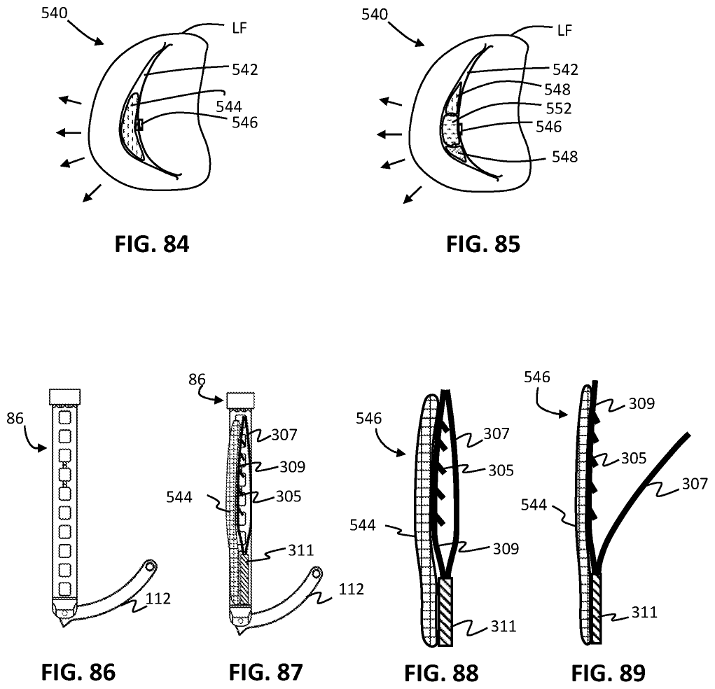

The expandable member 544 as seen in FIG. 84 is filling the gap between the leaflets sealing the gap and mitigating the regurgitation.

In an alternate embodiment, three or more spacers or expandable balloons 550, 552, 548 can be fitted in the space 542 between the leaflets (LF) as depicted in FIG. 85.

As explained in co-owned and reference application (PCT/US2017/042003), the fixation device is adaptable to both retrograde and antegrade configurations for deployment.

FIG. 86 is an exemplary embodiment of the release bar with an inverter 524 (referenced co-owned patents and in FIG. 23, PCT/0S2019/013853). The inverter is hinged to the release bar and therefore can swivel to enable ease of maneuver through the passage, along with a combination of configurations that may be used to manipulate the arms.

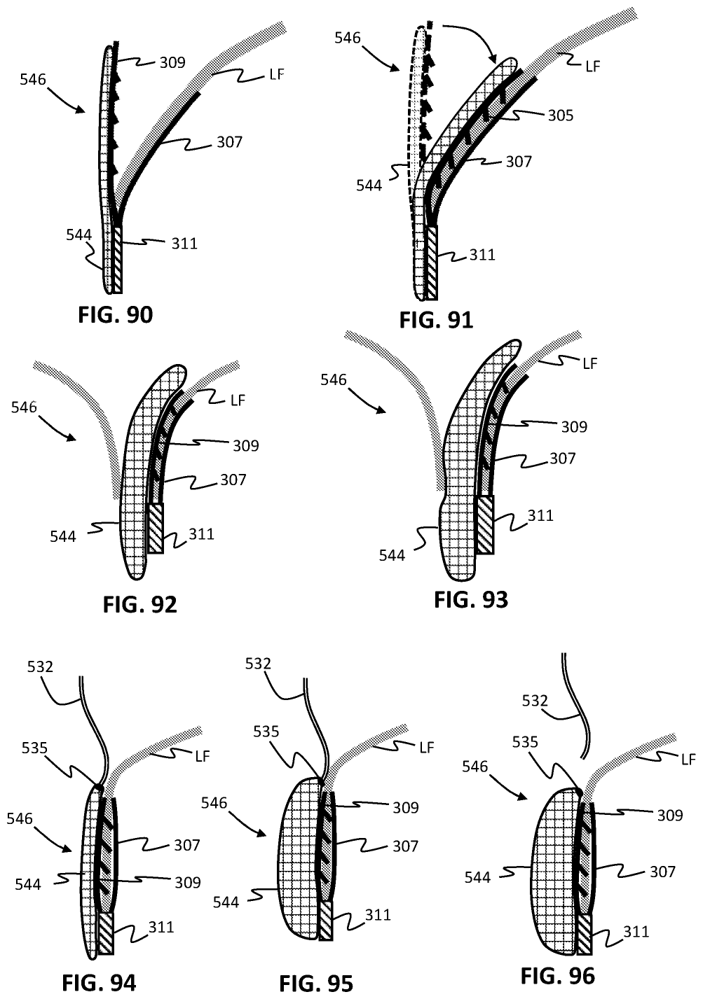

An exemplary expandable member 544 along with the inner arms 309 and outer arms 307 that are fastened at base 311, mounted on Release bar assembly 520 is shown in FIG. 87. The implant itself as shown in FIG. 88. In this exemplary embodiment, the inner arm 309 comprises of atraumatic barbs 305. The mechanism of grasping leaflets in FIGS. 90-105C is similar to that explained in co-owned patent US20200383782A1.

FIG. 89 shows the outer arm 307 in grasping angle. The leaflet has been grasped in-between the outer arm and inner arm in FIG. 90. In FIG. 91, the inner arm 309 has grasped the leaflet and the leaflet is being engaged by both the arms 307 and 309.

The expandable member 544 is inflated in FIG. 92. In an exemplary embodiment, the expandable member in FIG. 92 is rigid and less compliant. In an alternate exemplary embodiment, the FIG. 93 shows a more compliant and elastic in nature expandable member 544. It's shape dynamically changes to accommodate and engage the opposing leaflet.

FIG. 94 illustrates a detachable tether connected to the balloon or expandable member 544 via a port 535. Once the leaflet is grasped and implant is implanted, the tube 532 can remain attached to the implant and can be used to inflate the expandable member 544 externally/remotely. The inflation of the balloon can be increased or decreased to optimize reduction of MR, as shown in FIG. 95. Once optimized, the tube 532 can be detached from the self-sealing port 535, as shown in FIG. 96. US20180185185 incorporated here in full by reference, explains an exemplary self-sealing port design and how this tether can be detached.

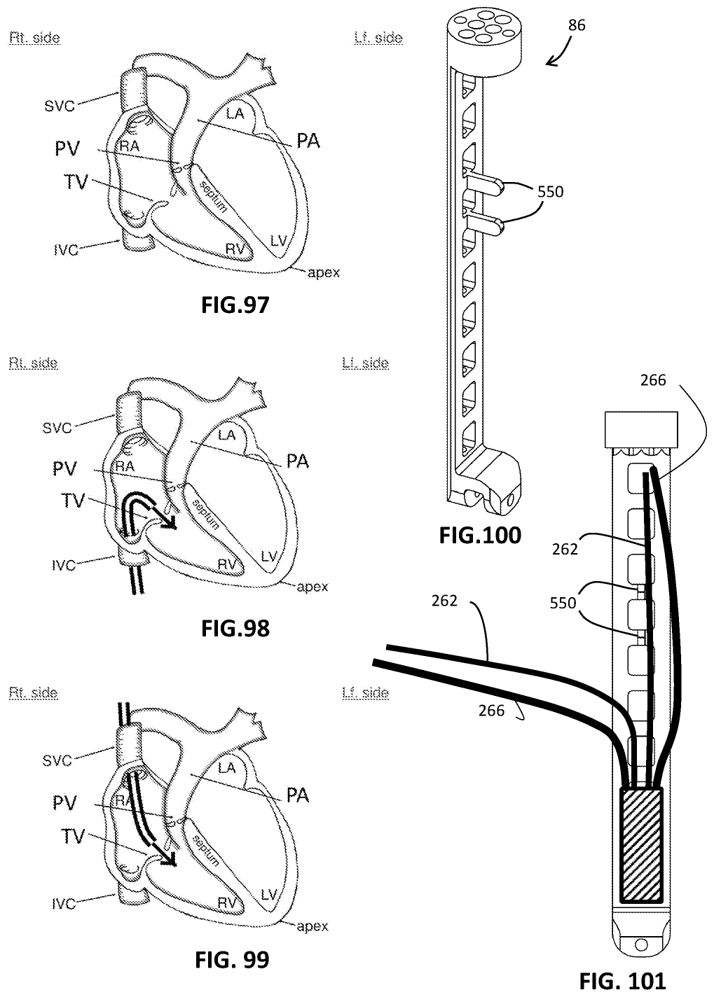

FIG. 97 illustrates the anatomical cross-section of a heart showing right atrium (RA) and right ventricle (RV). FIG. 98 illustrates the heart in FIG. 97 with a catheter inserted through the inferior vena cave (IVC) via the femoral vein access. Note the sharp U-turn the catheter needs to make to reach the tricuspid valve. FIG. 99 illustrates the heart in FIG. 97 with a catheter inserted through the superior vena cava (SVC) via the jugular vein access. Note the direct and less tortuous path from SVC to the tricuspid.

An exemplary embodiment in this invention can be configured to deliver a device via the SVC, using the jugular access.

An exemplary embodiment in this invention can be configured to deliver a device via the IVC, using the femoral access.

FIG. 100 shows an exemplary embodiment of the ReleaseBar with the center posts 550.

FIG. 101 illustrates an exemplary embodiment of a ReleaseBar component of the delivery system comprising of posts 550, which aid in partitioning each of the pair of the Grippers 262 and Arms 266 on their respective sides of the posts.

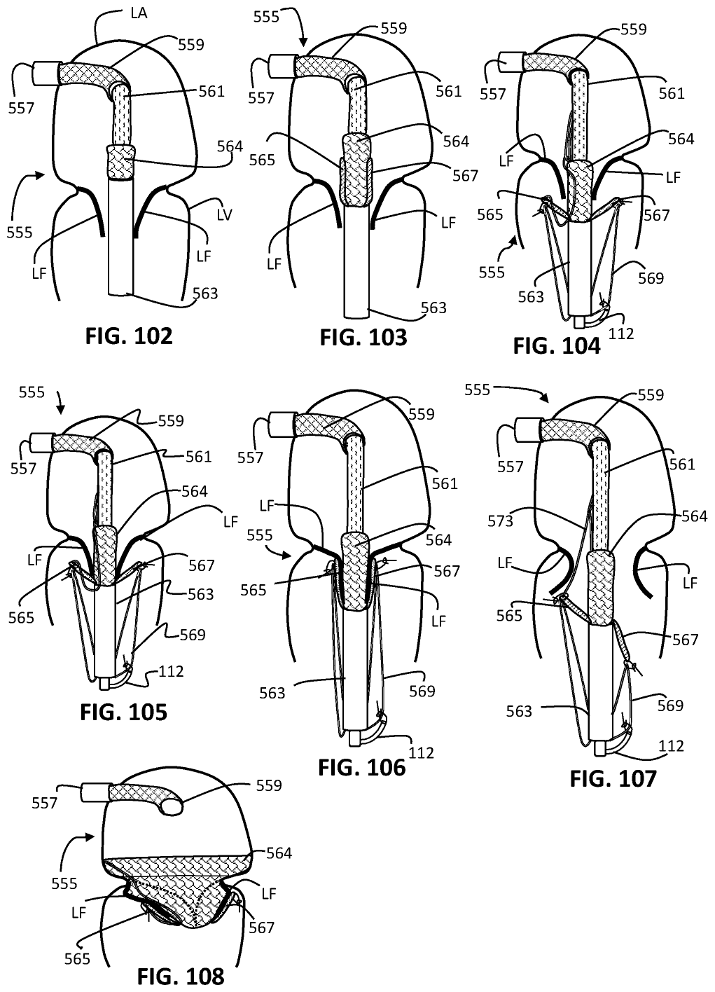

FIGS. 102 to 108 show an exemplary embodiment of heart valve replacement device 564, with the features of actuatable arms 565, 567 and/or grippers (not shown), akin to the device such as 260, 350 described in this and referenced co-owned patents.

In one exemplary method, the heart valve replacement device 564 is inserted into the human heart with a guide catheter 557 passed from the right atrium through a puncture in the atrial spectrum and into the left atrium LA. As shown in FIG. 102 the steerable guide catheter 557 has penetrated the atrial septum and entered the left atrium LA via steerable guide catheter 557. A delivery catheter 561 positions the heart valve replacement device 564 through the valve leaflets LF an into an upper region of the left ventricle, as shown in FIG. 103.

Distally advancing the device cover 563 exposes the device arms 567 and 565 from the stent's lateral positions in FIG. 103. The arms can be configured to self-expand or self-close radially. The number of arms depends on the number of leaflets in the heart valve and or the number of arms required to engage a single leaflet (LF).

The heart valve replacement device 564 is lowered to position the device arms 567 and 565 below the mitral valve plane. The arms 567 and 565 are then drawn by sutures 569 to a grasping angle as depicted in FIG. 104.

The leaflets LF are first stabilized by the arms 567 and 565, as shown in FIG. 105, and then captured or grasped when the arms are closed, as shown in FIG. 106.

If necessary, the leaflets LF may be disengaged as shown in FIG. 107. The leaflet on the left is disengaged from arm 565 with the help of the sutures 573 and left ventricular sutures 563. In an alternate embodiment, the right arm 567 is inverted with the help of the ventricular sutures attached to the inverter 112.

Once leaflet capture has been achieved, an expandable member of heart valve replacement device 564 may be released in the mitral valve annulus with the arms 565 and 567 anchoring the device in place, as shown in FIG. 108 As POSA will appreciate, the valve replacement device may be configured to minimize obstruction to the blood flow.

One particular advantage of this invention is that the method of grasping leaflets in the exemplary valve replacement device embodiment shown in FIGS. 102 to 108 can be similar to that explained in US20200383782A1. Furthermore, although not shown, in yet another alternate embodiment of the leaflet grasping mechanism my comprise of the Grippers, in addition to the arms as in US20200383782A1 and as will be evident to any Person of Ordinary Skill in the Art (POSA).

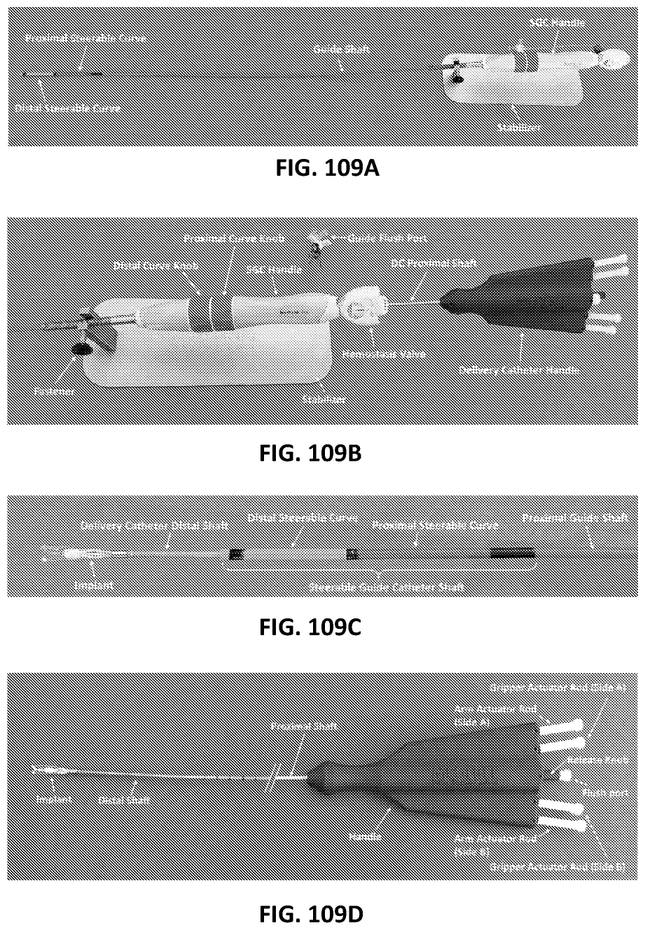

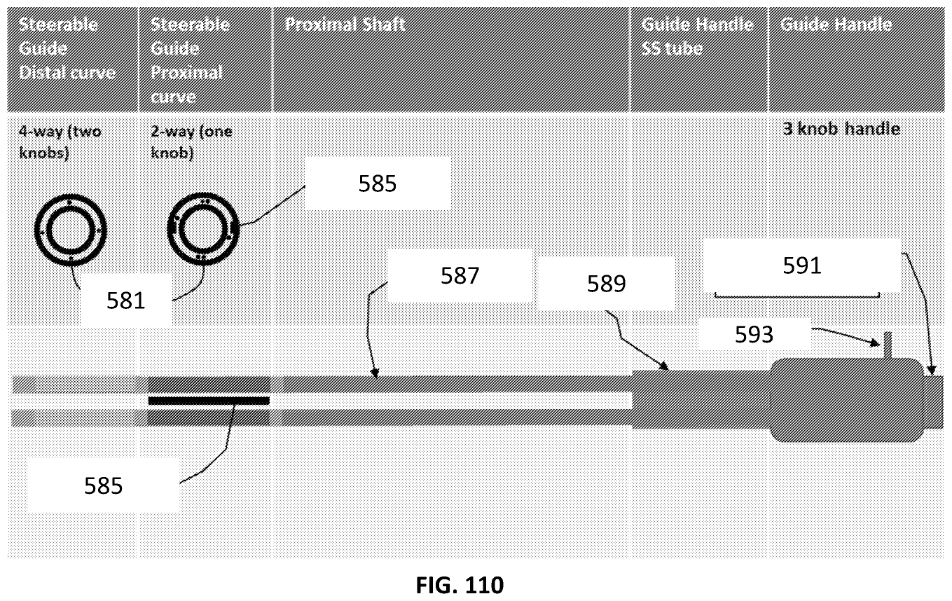

FIGS. 109A to 109D show an exemplary embodiment of VALVE REPAIR SYSTEM, comprising of Stabilizer, Steerable Guide Catheter, Device, Device Delivery Catheter, that builds and based on co-owned and referenced patents, including WO2019143726A. While the exemplary handle in FIG. 109D only shows flush port in addition to Arm, Gripper actuator rods, separate actuator rods dedicated for bailout may be easily incorporated. Further, each of the actuator rod may perform more than one functions, manually, semi-automatically, automatically, and or robotically.

FIG. 110 shows an exemplary embodiment of the Steerable Guide Catheter. In particular, it shows exemplary straight stiffening members placed diametrically opposite ends, thereby, stiffing the catheter in its orthogonal plane, while allowing the catheter to bend in along its plane, as controlled by the pull-wires. Although straight stiffening members have been shown in this example, it will be quite obvious to POSA that the stiffening members may be placed in curved or helical or any other pattern, in series, parallel, intermittent or continuous, stent-like patterned cuts in a tube, to achieve desired steering or bending characteristics at a given section or length of the catheter. Further, the stiffening members of same or varying stiffness may be formed of metal, polymer, ceramic, composite, fiber, and/or simply by adding or removing material from the shaft wall. Pull wires can be rectangular, square, round or any other shapes known to POSA.

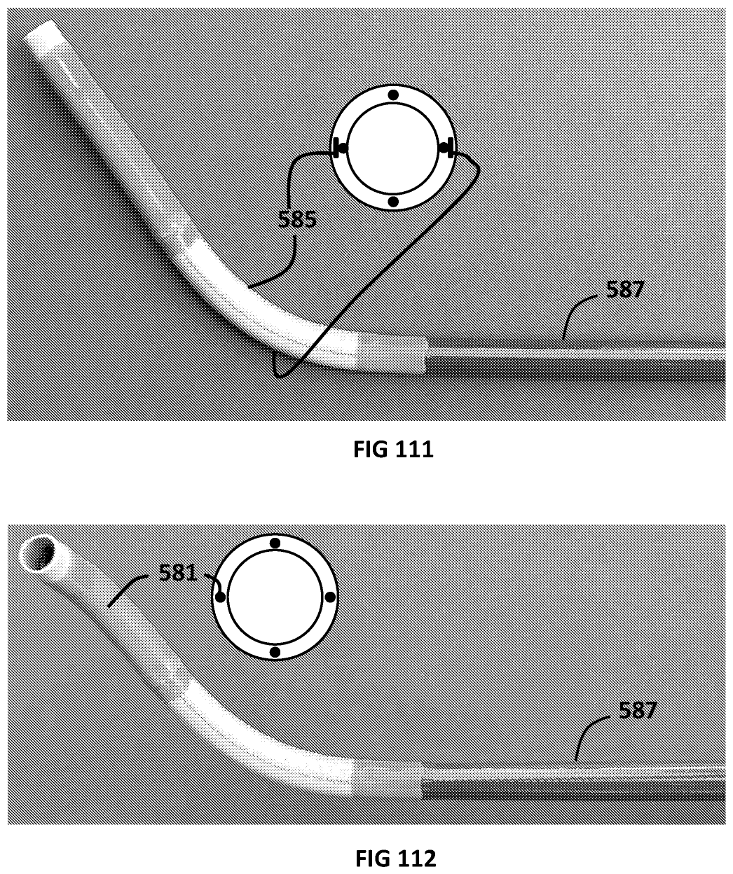

In one exemplary embodiment as shown in FIG. 111, the pull wires in the proximal curve region comprise of rectangular stiffener/strips 585, as they can serve as stiffeners too.

In one exemplary embodiment as shown in FIG. 111, the pull-wires in the proximal curve region comprise of rectangular stiffener/strips 585, as they can serve as stiffeners too.

In one exemplary embodiment as shown in FIG. 112, the pull wires in the distal curve region comprise of circular pull-wires 581 in all 4 directions, to allow for 4-way steering.

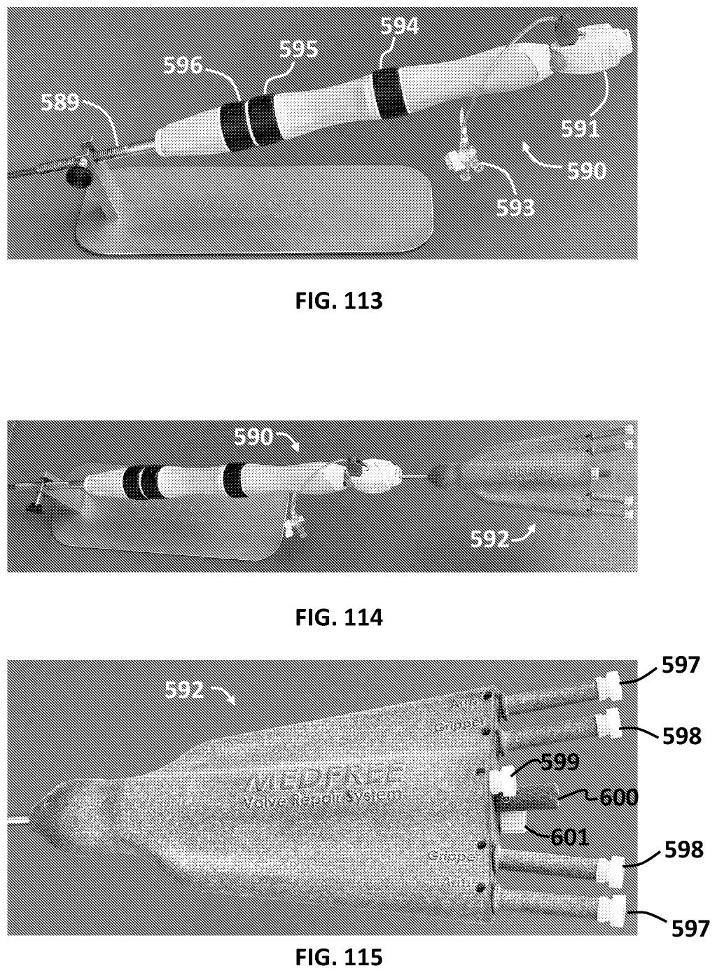

FIG. 113 shows an exemplary embodiment of a Steerable Guide Catheter (SGC) 590 mounted on an exemplary Stabilizer. The proximal knob 594 is configured to provide 2-way steering of the proximal curved segment as in FIG. 111, using rectangular pull-wires. The middle knob 595 is configured to provide 2-way steering in the anterior/posterior direction, while the distal knob 596 provides 2-way steering in the medial/lateral direction, as shown in FIG. 112.

FIG. 114 shows an exemplary configuration of Valve Repair System, comprising of the SGC 593 and Delivery Catheter (DC) 592, mounted on a Stabilizer.

FIG. 115 shows an exemplary embodiment of DC handle. As can be seen in FIG. 109D, the plane of the implant matches the plane of the handle. Further, owing to torsional rigidity of the delivery catheter, each pair of Actuator rod-Arm 597 and Actuator rod-Gripper 598 match their respective implant arm and gripper pairs, for intuitive operation and ease of use.

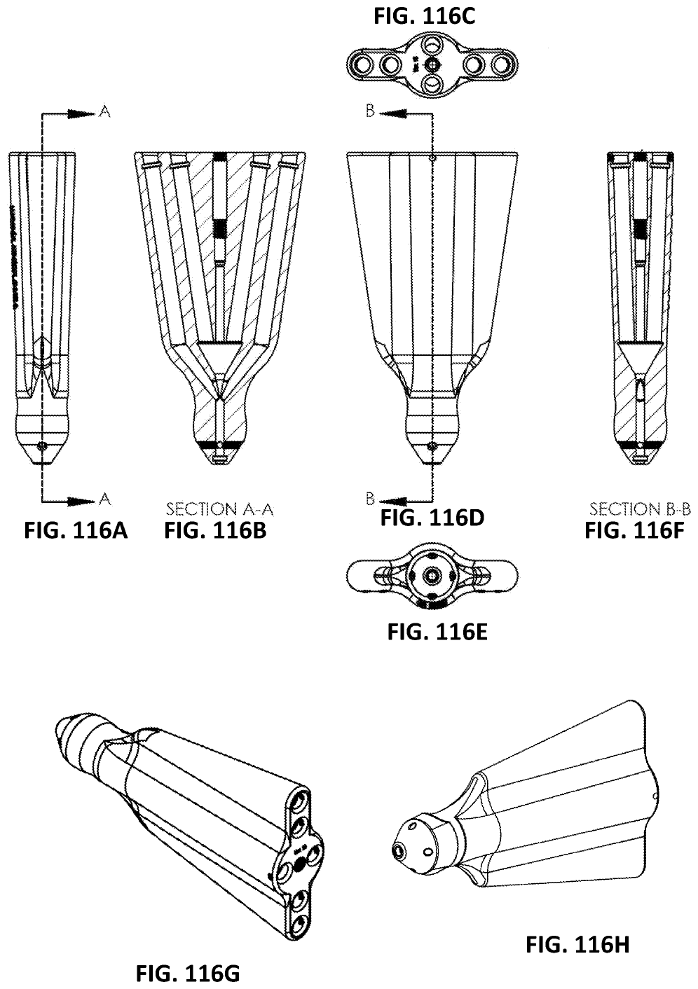

FIGS. 116A to 116H show various views of the DC handle without actuator rods, flush port or release knob.

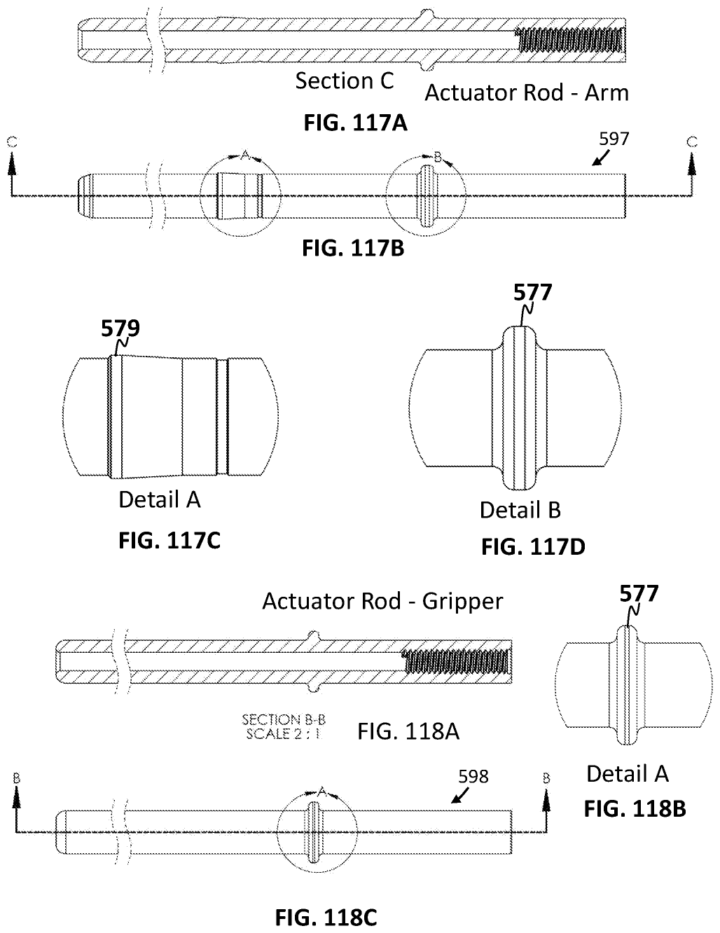

FIGS. 117A to 117D show various views of the Actuator rod-arm 597.

FIGS. 118A to 117C show various views of the Actuator rod-gripper 597.

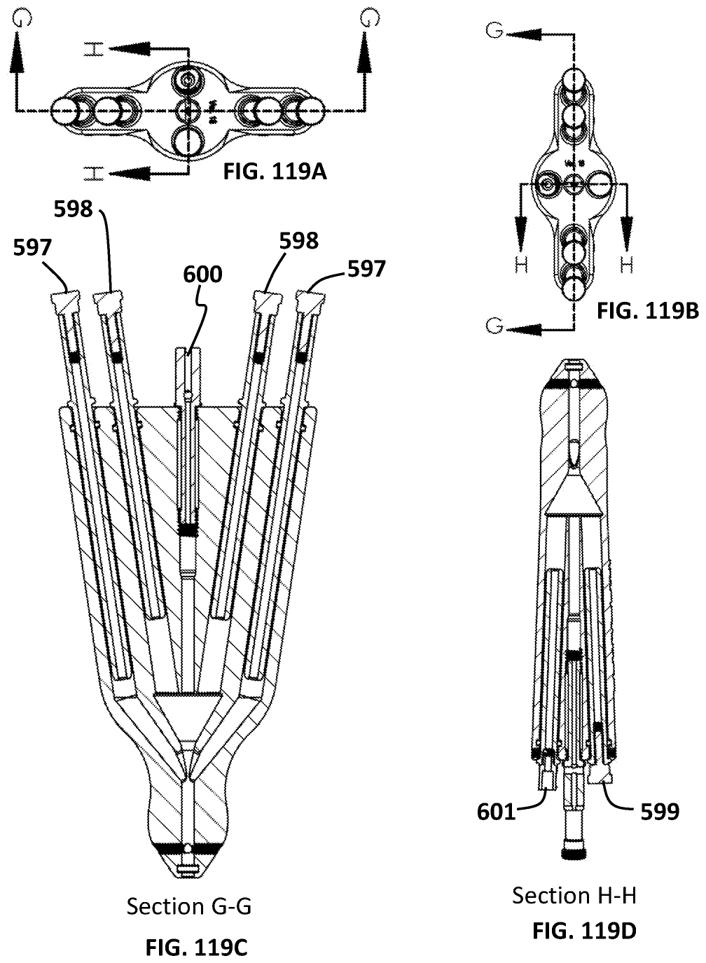

FIGS. 119A to 119D show various views of the DC handle. The actuating sutures are connected to their corresponding actuator rods, while the Release Knob is attached to the Release mandrel, similar to the description provided in the referenced and co-owned patents.

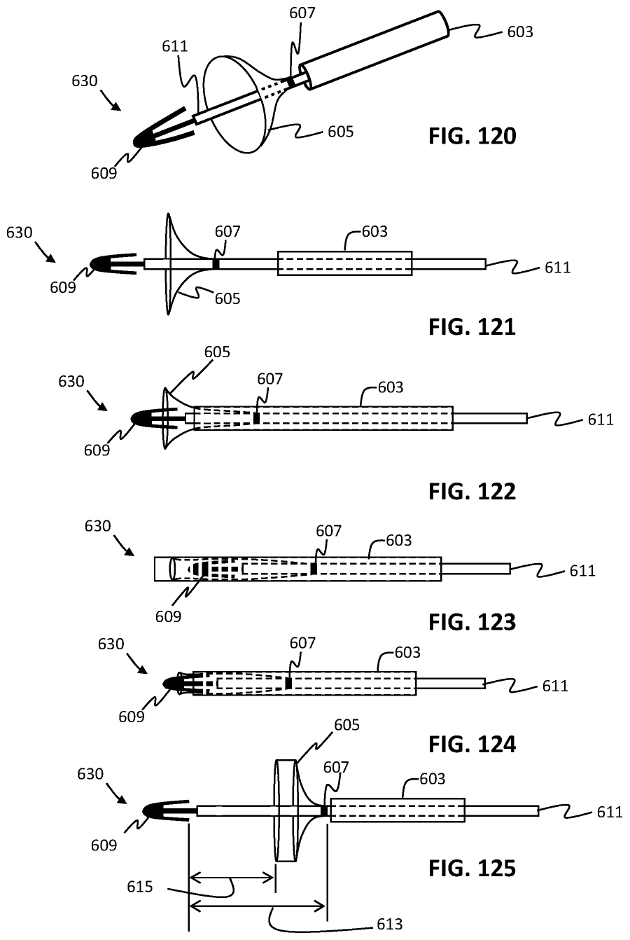

In an exemplary embodiment as shown in FIG. 120, a funnel shaped mesh 605 made of nitinol wires or laser cut tube. It is configured to expand into a funnel or any other shape that has a larger diameter than the guide catheter 603 that it protrudes out. The larger diameter aids in capture of the implant arms into the guide catheter 603. Thus, allowing complete retrieval of the implant and mitigate the problem of the implant's arms getting stuck in the distal tip of the guide catheter 603, which is a common problem with competitor device such as the MitraClip® The mesh 609 is bonded to the delivery catheter 611 at a site 607 proximal to the implant.

FIG. 121 shows side view of the funnel shaped mesh, in a configuration wherein it is completely outside of the catheter 603. The funnel shaped mesh expands as it is pushed outside of the guide catheter 603 and contracts as it is pulled into the guide catheter 603. The funnel shaped mesh expands and foreshortens to stay proximal to the implant and is configured to avoid interference with the valve leaflets and the implant.

FIG. 122 illustrates the funnel shaped mesh 605 capturing the implant 609.

FIG. 123 shows the funnel shaped mesh completely covering the implant and FIG. 124 shows the funnel shaped mesh partially covering the implant, while inside the guide catheter 603. The implant could either be fully covered by the mesh 605 or be partially covered but either way the mesh 605 at minimum encompasses the arms of the implant. That is, the broadest free aspects of the implant, which the tip of arms of the implant are fully encapsulated by the covering 605.

The distance shown in FIG. 125 as 615 represents the possible distance between the implant and the fully expanded funnel 605. The distance 615 is preferably 7.5 cm and can configured to be 0, 1, 2, 3, 4, 5, 6, . . . , 199, and or 200 cm. The distance 613 represents the possible distance between the implant 609 and the point of bonding 607 between the funnel 605 and the delivery catheter 611. It could be configured to be about 0, 1, 2, 3, 4, 5, 6, . . . , 198, 199, and or 200 cm.

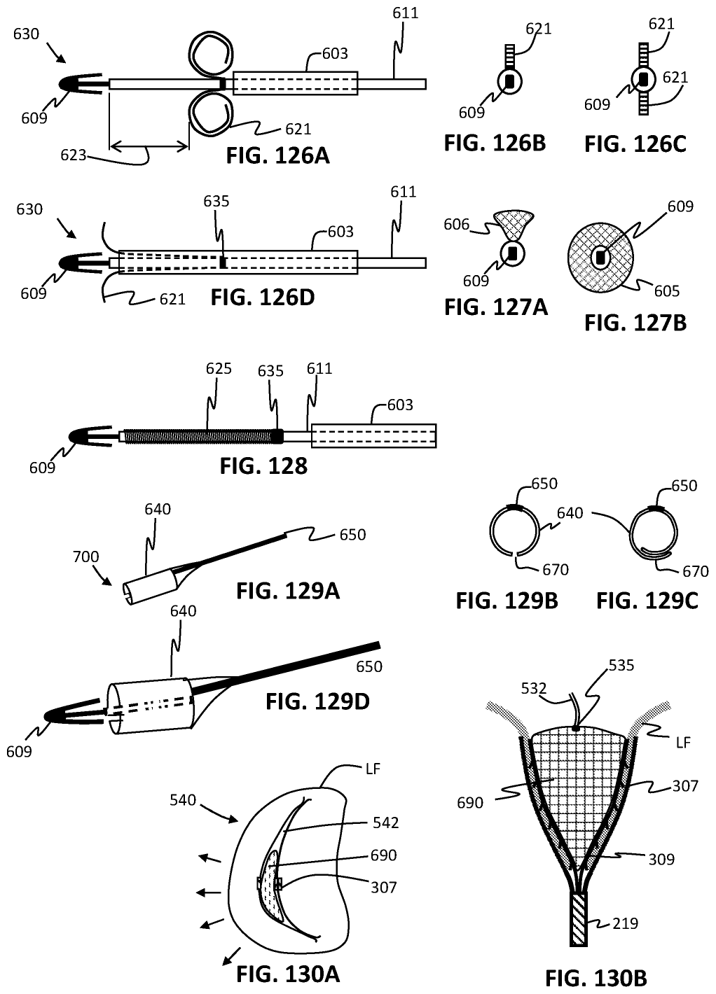

FIG. 126A illustrates an exemplary embodiment of this invention comprising of a coiled leaf spring instead of the funnel shaped mesh described in FIGS. 120 to 125. A 360-degree funnel won't be required for an implant with just two laterally placed arms. The main function of 621 is the same as funnel 605, which is to guide the protruded arms back into the guide catheter for complete retrieval and bail out. An elastic or super elastic strip that can be rolled into a coil can be used instead of the funnel in such cases. The distance 623 represents the distance between the implant and the coiled leaf spring. It could be about 0, 1, 2, 3, 4, 5, 6, . . . , 199, and or 200 cm (preferably 7.5 cm). FIG. 126B illustrates the orthogonal cross-sectional view of FIG. 126A with just one coiled leaf spring 621. If the implant has just one arm, a single coiled leaf spring is enough to pull the device out. FIG. 126C illustrates the orthogonal cross-sectional view of FIG. 126A with both the coiled leaf springs. If the implant has two arms, two coiled leaf springs would be required to pull the device out.

FIG. 126D shows the coiled leaf spring 621 almost completely uncoiled and ready to grasp the implant 609. Once the implant has been grasped, it is pulled into the guide catheter and can be completely retrieved. FIG. 127A shows a fan shaped mesh 606 instead of a funnel 605 for implant retrieval. A complete 360-degree fan 605 resembles a cone that can also be used depending on the type of the implant. FIG. 127B shows the orthogonal view of FIG. 121 with the funnel shaped mesh 605 fully expanded and the implant 609 in the middle.

One advantage of the exemplary expanding funnel, coil, and/or fan features is that once expanded post trans-septal crossing of the guide catheter, they provide safeguard against accidental pullout of the guide catheter.

The delivery catheter has a coiled spring attached to it at the distal segment, just proximal to the implant attachment, as shown in FIG. 128. The coiled spring facilitates easy maneuver of the implant 609 through tight curves as well as aids in maintaining straightness of the shaft, as it exits out of the guide catheter, improving ease of use. The coil spring can be manufactured from wire or laser cut tube.

FIG. 129A shows an exemplary rescue catheter 700 with a slit 670. The rescue catheter can be used to capture the implant, as previously described in co-owned application.

FIG. 129B show alternate embodiments of the cross-sectional view of the exemplary rescue catheter 700.

In FIG. 129C the slit ends of 640 are overlapping on each other and configured to ride snugly over the guide catheter.

Alternatively, the rescue catheter can be configured to ride over the delivery catheter and inside the guide. Further, it is configured expand upon exiting the guide catheter to capture the implant.

FIG. 130A shows atrial view of a mitral valve schematic with an exemplary implant as shown in FIG. 130B, comprising of a pair of inner and outer arms and an expandable feature 690 in between the two inner arms. The expandable feature 690 may be a balloon placed in between two inner arms, and maybe connected to one or both inner arms or to outer base. The balloon may be configured as a spacer to mitigate regurgitation. Furthermore, the spacer balloon size may be adjustable via controlled inflation or deflation, remotely using a detachable tube 532, as explained previously in FIGS. 94, 95, and 96. Over a period of time, after the balloon has engaged the leaflets, the balloon maybe configured to deflate slowly. This slow and progressive cinching over a period of time is advantageous, as aggressive acute leaflet cinching may cause leaflet tears, which is a problem in the competitive MitraClip® device. Slow deflation spacer for controlled cinching overtime may be achieved via a) diffusion, degradation, displacement, and remotely via a detachable tube 532. Furthermore, the entire expandable feature 690 maybe detachably attached to the implant.

1. An endovascular heart valve repair system comprising:
a delivery catheter having a distal end configured to be introduced into a heart chamber adjacent to a pair of coapting heart valve leaflets,
a valve repair leaflet grasping device attached to a distal region of the delivery catheter and comprising a first pair of leaflet capture arms including a first inner arm and a first outer arm and a second pair of leaflet capture arms comprising a second inner arm and a second outer; and
an inverter bar positioned on the deliver catheter distally of the valve repair leaflet device, said inverter bar oriented transversely relative to a longitudinal axis of the delivery catheter; and
a bailout suture passing over an exterior of the distal region of the delivery catheter and looped through opposite lateral ends of the inverter bar so that two ends of the bailout suture may be drawn proximally to create a triangular cage to exclude valve leaflets from the valve repair leaflet grasping device.

2. An endovascular heart valve repair device as in claim 1, further comprising a secondary suture loop positioned over the bailout suture loop and configured to be drawn proximally to cinch the bailout suture toward the delivery catheter to allow the valve repair leaflet grasping device to grasp the valve leaflets.

3. An endovascular heart valve repair device as in claim 2, wherein the secondary suture is restrained in a secondary structure with a free loop end passing out of the secondary structure and capturing a distal loop segment of the bailout loop.

4. A method for expelling valve leaflets from a valve repair leaflet grasping device, said method comprising:
deploying a first length of bailout suture across a first inner arm and a first outer arm of a first pair of leaflet capture arms and a second length of bailout suture across second inner arm and a second outer arm of a second pair of leaflet capture arms, wherein the suture lengths exclude the leaflets from the space between each pairs of arms.

5. A method as in claim 4, further comprising deploying a secondary suture loop to radially constrain the bailout suture so that at least one of the first and second pairs of leaflet capture arms are able to capture leaflets.

6. A method as in claim 5, wherein deploying the secondary suture loop radially constrains the bailout suture so that both of the first and second pairs of leaflet capture arms are able to capture leaflets.

7. An endovascular heart valve repair system comprising:
a delivery catheter having a distal end configured to be introduced into a heart chamber adjacent to a pair of coapting heart valve leaflets;
a valve repair leaflet grasping device comprising a hub configured to be removably attached to the delivery catheter, a first pair of leaflet capture arms comprising a first inner arm and a first outer arm coupled to the hub, and a second pair of leaflet capture arms comprising a second inner arm and a second outer arm coupled to the hub, wherein the first and second pairs of leaflet capture arms together form a cleft on their atrial sides above the hub; and
a spacer disposed over the cleft to inhibit thrombus formation.

8. An endovascular heart valve repair device as in claim 7, wherein the spacer comprises one or more of an expandable sponge, a compressible sponge, a mesh, a balloon, or anon-thrombogenic fabric.

9. An endovascular heart valve repair device as in claim 7, wherein the spacer is fastened to an atrial side of the hub and/or pair of leaflet capture arms using suture, bond, weld, glue, a fastener.

10. An endovascular heart valve repair device as in claim 7, comprising two spacers where each spacer is fastened to a pair of leaflet capture arms.

11. An endovascular heart valve repair device as in claim 7, further comprising spacers attached to a ventricular side of each pair of leaflet capture arms.

12. An endovascular heart valve prosthesis comprising:
a peripheral scaffold configured to be expanded within an annulus of a patient's native heart valve;
one or more arms disposed on an outer peripheral surface of the peripheral scaffold, said arms configured to be clipped over free ends of valve leaflets of the patient's heart valve;
prosthetic valve leaflets coupled to an interior surface of the peripheral scaffold.

13. An endovascular heart valve prosthesis as in claim 12, wherein comprising at least two arms positioned on the peripheral scaffold to engage anterior and posterior leaflets on a patient's mitral valve.

14. An endovascular heart valve prosthesis as in claim 12, wherein at least some of the arms comprise a pair of leaflet capture arms including an inner arm configured to engage an atrial side of a leaflet and an outer arm configured to engage a ventricular side of a leaflet.

15. An endovascular heart valve prosthesis as in claim 12, wherein the peripheral scaffold is self-expanding.

16. An endovascular heart valve prosthesis as in claim 12, wherein the peripheral scaffold is balloon expandable.

17. A method for deploying an endovascular heart valve in a patient's native heart valve annulus, said method comprising:
positioning a peripheral scaffold within the annulus of a patient's native heart valve in a radially collapsed configuration;
attaching one or more arms disposed on an outer peripheral surface of the peripheral scaffold over free ends of one or more valve leaflets of the patient's heart valve; and
expanding the peripheral scaffold within the annulus of a patient's native heart valve to a radially expanded configuration.

18. A method as in claim 17, positioning comprises transseptally advancing a delivery catheter into the patient's left atrium and advancing the endovascular heart valve through the leaflets toward the patient's ventricle.

19. A method as in claim 17, wherein attaching one or more arms comprises opening one or more resiliently mounted arms on the peripheral scaffold and allowing said arms to self-close to capture the valve leaflets.

20. A method as in claim 19, wherein at least some of the arms comprise a pair of leaflet capture arms including an inner arm configured to engage an atrial side of a leaflet and an outer arm configured to engage a ventricular side of a leaflet, wherein the inner and outer arms are separately opened and closed over the valve leaflets.
